(19) United States
(12) Patent Application Publication
(10) Pub. No.: US 2010/0048606 A1
(43) Pub. Date: Feb. 25, 2010

(54) 10-Substituted Cytisine Derivatives and Methods of Use Thereof

(75) Inventors: Alan P. Kozikowski, Chicago, IL (US); Werner Tueckmantel, Yorktown Heights, NY (US); Sheela Chellappan, Chicago, IL (US); Kenneth J. Kellar, Bethesda, MD (US); Yingxian Xiao, Potomac, MD (US)

Correspondence Address: FOLEY HOAG, LLP;PATENT GROUP, WORLD TRADE CENTER WEST, 155 SEAPORT BLVD, BOSTON, MA, 02110, US

(73) Assignee: Georgetown University Office of Technology Commercialization, Washington, DC (US)

(21) Appl. No.: 12/295,157

(22) PCT Filed: Mar. 29, 2007
(86) PCT No.: PCT/US07/65498

§ 371 Date: Nov. 4, 2009

Related U.S. Application Data
(60) Provisional application No. 60/786,907, filed on Mar. 29, 2006.

(51) Int. Cl. A61K 31/439; C07D 471/18

(52) U.S. Cl. 514/292; 546/81

(57) ABSTRACT

The present invention relates to substituted cytisine compounds that are useful in treating diseases impacted by a nicotinic ACh receptor. One aspect of the invention relates to 10-substituted cytisine compounds. In certain instances, the cytisine is substituted in the 10-position by an alkyl, aryl or aralkyl group. The present invention also relates to a pharmaceutical composition comprises the substituted cytisine compound or the 10-substituted cytisine compound. The invention also relates to a method of modulation a nicotinic ACh receptor in a mammal, comprising the step of administering to a mammal in need thereof a therapeutically effective amount of a substituted cytisine. In certain instances, the substituted cytisine is a 10-substituted cytisine. Another aspect of the present invention relates a method of treating a disease impacted by a nicotinic ACh receptor, comprising the step of administering to a mammal in need thereof a therapeutically effective amount of a substituted cytisine. In certain instances, the substituted cytisine is a 10-substituted cytisine.

### RELATED APPLICATIONS

This application claims the benefit of priority to U.S. Provisional Patent Application Ser. No. 60/786,907, filed Mar. 29, 2006.

### GOVERNMENT SUPPORT

This invention was made with support provided by the National Institutes of Health (Grant No. R01 DA017980); therefore, the government has certain rights in the invention.

### BACKGROUND OF THE INVENTION

The endogenous cholinergic neurotransmitter, acetylcholine (ACh), exerts its biological effect via two types of cholinergic receptors: the muscarinic acetylcholine receptors (mAChRs) and the nicotinic ACh receptors (nAChRs). The muscle type nAChR is localized at the neuromuscular junction and is the target of several clinically used muscle relaxants. nAChRs can be found throughout the central and peripheral nervous system and are important therapeutic targets for treating neurodegenerative disorders and other CNS disorders, including Alzheimer's disease, Parkinson's disease, Tourette's syndrome, schizophrenia, attention deficit disorder, anxiety, pain and drug addiction.

Alzheimer's disease is characterised by a profound loss of memory and cognitive function caused by a severe depletion of cholinergic neurons, i.e., neurons that release acetylcholine. A reduction in the number of nicotinic ACh receptors is observed with the progression of Alzheimer's disease. It is believed that the neurons in the cortex die due to lack of stimulation of the nicotinic ACh receptors. Further, it is predicted that treatment of Alzheimer patients with nicotinic ACh receptor modulators will not only improve the patients' memory, but also act to keep these neurons alive.

Degeneration of the cholinergic system, as observed with Alzheimer's disease, has been found with other diseases and conditions. For example, the dementia and cognitive impairment due to brain damage related to alcoholism is associated with degeneration of the cholinergic system. Healthy aged-adults and aged-rats have also been shown to suffer from degeneration of the cholinergic system, suggesting that the cholinergic system is implicated in memory disturbances suffered by aged animals and humans. It follows, therefore, that compounds which modulate nicotinic ACh receptors may be useful in the treatment of Alzheimer's disease, memory loss, memory dysfunction, AIDS-dementia, senile dementia and neurodegenerative disorders.

Parkinson's disease is a neurodegenerative disease that affects a patient's movement and coordination. Loss of nicotinic receptors associated with dopaminergic neurons is one of the symptoms of Parkinson's disease. It is postulated that administration of a compound that modulates the nicotinic receptor may ameliorate the symptoms of Parkinson's disease because nicotine administration increases the number of nicotinic receptors. Since it is possible that the loss of nicotinic receptors associated with dopaminergic neurons may interfere with dopamine release, other conditions associated with deficiencies in the dopaminergic system (such as drug addiction, depression, obesity and narcolepsy) may be implicated.

Compounds that modulate nicotinic ACh receptors may be useful in treating Tourette's syndrome and schizophrenia. Tourette's syndrome is a neuropsychiatric disorder involving a range of neurological and behavioral symptoms. It is believed that neurotransmitter dysfunction is involved and that nicotine will be beneficial in the treatment of the disease (Devor et al. The Lancet, vol. 8670 p. 1046, 1989). Schizophrenia is a severe psychiatric illness. Neuroleptic compounds have been used to treat the disease; the effect of the compounds is believed to involve an interaction with the dopaminergic system Nicotine is proposed to be effective in the treatment of schizophrenia (Merriam et. al. Psychiatr. annals, vol. 23, p. 171-178, 1993 and Adler et al. Biol. Psychiatry, vol. 32, p. 607-616, 1992).

Nicotine has been reported to have an effect on neurotransmitter release in several systems. Release of acetylcholine and dopamine by neurons upon administration of nicotine has been reported (J. Neurochem. vol. 43, p. 1593-1598, 1984), as well as release of norepinephrine by Hall et al. (Biochem. Pharmacol. vol. 21, p. 1829-1838, 1972), release of serotonin by Hery et al. (Arch. Int. Pharmacodyn. Ther. vol. 296. p. 91-97, 1977), and release of glutamate by Toth et al. (Neurochem. Res. vol. 17, p. 265-271, 1992). The serotonin system and dysfunctions of the serotonergic system are believed to be involved in diseases or conditions like anxiety, depression, eating disorders, obsessive compulsive disorder, panic disorders, chemical substance abuse, alcoholism pain, memory deficits and anxiety, pseudodementia, Ganser's syndrome, migraine pain, bulimia, obesity, pre-menstrual syndrome or late luteal phase syndrome, tobacco abuse, post-traumatic syndrome, social phobia, chronic fatigue syndrome, premature ejaculation, erectile difficulty, anorexia nervosa, disorders of sleep, autism, mutism, and trichotillomania.

Compounds that modulate nicotinic ACh receptors may be useful for improving concentration or reducing the side effects of withdrawal from addictive substances, such as tobacco. Nicotine improves concentration and task performance. Therefore, compounds exhibiting nicotine receptor modulating properties are likely to be useful in treating learning deficit, cognition deficit, attention deficit disorder, attention deficit hyperactivity disorder, and dyslexia. Tobacco use, especially cigarette smoking, is recognised as a serious health problem. However, nicotine withdrawal symptoms associated with smoking cessation make it difficult to break this habit. Withdrawal symptoms include anger, anxiety, difficulties in concentrating, restlessness, decreased heart rate and increased appetite and weight gain. Nicotine itself has been shown to ease the withdrawal symptoms. Moreover, as the addictive properties of tobacco products are due to the nicotine contained therein, nAChRs also become important targets for the discovery of medications for use in smoking cessation. See R. C. Hogg et al. Curr. Drug. Targets. CNS Neurol. Disord. vol. 3, p. 123-130, 2004; F. Clementi et al. Trends Pharmacol. Sci. p. 21, p. 35-37, 2000; K. J. Kellar et al. Nicotine Tob. Res. vol. 1, p. S117-120, 1999; and J. W. Daly Cell. Mol. Neurobiol. vol. 25, p. 513-551, 2005. Withdrawal from other addictive substances, e.g., opiates, benzodiazepines, ethanol, tobacco or nicotine, is generally a traumatic experience characterized by anxiety and frustration. Nicotine has been found to be effective in reducing anger, irritability, frustration and feelings of tension without causing general response depression, drowsiness, or sedation and compounds having similar characteristics as nicotine are likely to have similar effects.

A need exists for analgesic compounds with reduced side effects which can relieve mild, moderate and severe pain of acute, chronic or recurrent character as well as migraine pain, postoperative pain, and phantom limb pain. Mild to moderate pain is normally treatable with NSAID's (non-steroidal anti-inflammatory drugs) while opiates are used preferentially for moderate to severe pain. However, opiates have some well-known side-effects, including chemical dependence, potential for abuse, and a depressive effect on the respiratory and gastrointestinal system. Epibatidine, a compound isolated from the skin of a poison frog, is a very potent analgesic with a potency of approximately 500 times that of morphine. The analgesic effect is not affected by naloxone, which is an indication of a negligible affinity for the opiate receptors. Epibatidine is a nicotinic cholinergic receptor agonist, and it is therefore very likely that compounds possessing this receptor modulating characteristic will also show a strong analgesic response. It is well known that nicotine has an effect on appetite, and it is predicted that modulators at the nicotinic ACh receptor may be useful as appetite suppressants in the treatment of obesity and eating disorders.

Cholinergic receptors play an important role in the functioning of muscles, organs and generally in the central nervous system. There are also complex interactions between cholinergic receptors and the function of receptors of other neurotransmitters, such as dopamine, serotonin and noradrenaline. It is likely that nicotinic receptor modulator compounds can be effective in preventing or treating conditions or disorders or diseases like inflammation, inflammatory skin conditions, Chron's disease, inflammatory bowel disease, ulcerative colitis, diarrhoea, neurodegeneration, perpherical neuropathy, amyotrophic lateral sclerosis, nociception, endocrine disorders, thyrotoxicosis, pheochromocytoma, hypertension, arrhythmias, mania, manic depression, Huntington's disease, and jetlag.

Although a number of diseases are linked to neuronal nicotinic acetylcholine receptor activity, treatment options are complicated by the fact that there are several neuronal nicotinic acetylcholine receptor subtypes. Neuronal nicotinic acetylcholine receptors (nAChRs) belong to a heterogeneous family of pentameric ligand-gated ion channels which are differently expressed in many regions of the central nervous system (CNS) and peripheral nervous system. See M. W. Holladay et al. J. Med. Chem. vol. 40, p. 4169-4194, 1997; A. Karlin Nat. Rev. Neurosci. vol. 3, p. 102-114, 2002; and A. A. Jensen et al. J. Med. Chem. vol. 48, p. 4705-4745, 2005. In the CNS, nAChRs regulate transmitter release, cell excitability, and neuronal integration.

The nAChRs are comprised of various combinations of different subunits, of which seventeen (α1-α10, β1-β4, γ, δ and ε) are presently known. Different subunit combinations define the various nAChR subtypes. Further, different receptor subtypes have characteristic pharmacological and biophysical properties, as well as different locations within the nervous system. See N. S. Millar Biochem. Soc. Trans. vol. 31, p. 869-874, 2003.

Therefore, the need exists for nAChR ligands that are selective for the various nicotinic ACh receptors. See M. W. Holladay et al. J. Med. Chem. vol. 40, p. 4169-4194, 1997 and G. K. Lloyd et al. J. Pharmacol. Exp. Ther. vol. 292, p. 461-467, 2000. Therapeutic agents that are selective for certain nicotinic ACh receptor subtypes would be highly valuable because they could increase both the safety and efficacy of the therapeutic agent.

### SUMMARY OF THE INVENTION

One aspect of the present invention relates to 10-substituted cytisine compounds. In certain instances, the cytisine is substituted at the 10-position by an alkyl, aryl, or aralkyl group. In certain instances, the 10-substituted cytisine compound has a Kᵢ of less than about 25 nM in an assay based on an α4β2 nAChR receptor. In certain instances, the cytisine is substituted at the 10-position by a methyl or hydroxymethyl group. Another aspect of the present invention relates to a pharmaceutical composition comprising a 10-substituted cytisine compound. Another aspect of the present invention relates to a method of modulating a nicotinic ACh receptor in a mammal, comprising the step of administering to a mammal in need thereof a therapeutically effective amount of a 10-substituted cytisine. In certain instances, the mammal is a human. Another aspect of the present invention relates to a method of treating a disease impacted by a nicotinic ACh receptor, comprising the step of administering to a mammal in need thereof a therapeutically effective amount of a 10-substituted cytisine. In certain instances, said disease impacted by a nicotinic ACh receptor is selected from the group consisting of Alzheimer's disease, Parkinson's disease, dyskinesias, Tourette's syndrome, and schizophrenia.

### BRIEF DESCRIPTION OF THE FIGURES

FIG. 1 depicts the structure of a neuronal nicotinic acetylcholine receptor (nAChR).

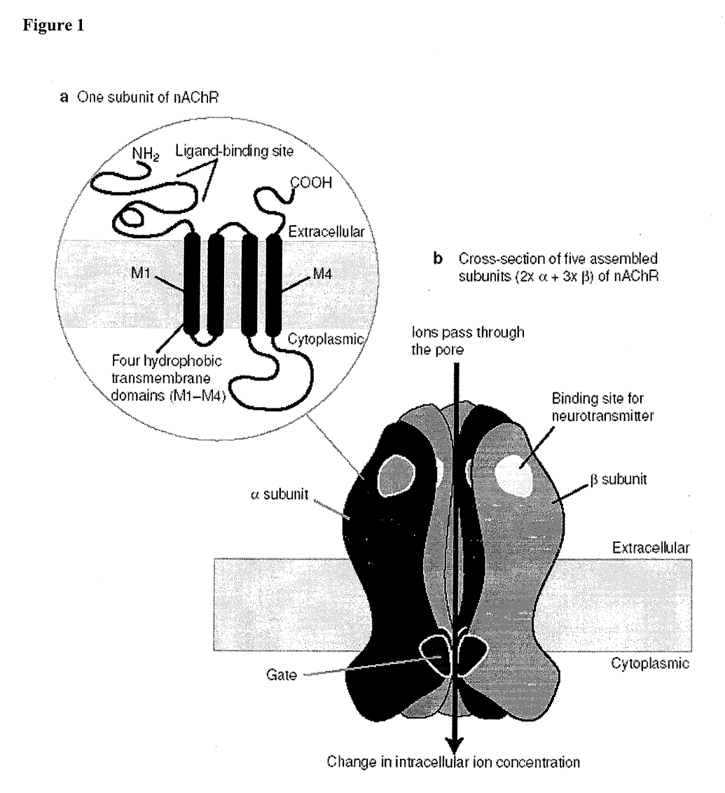

FIG. 2 depicts the structure of (−)-nicotine, cytisine, (−)-epibatidine, and compound A-84543.

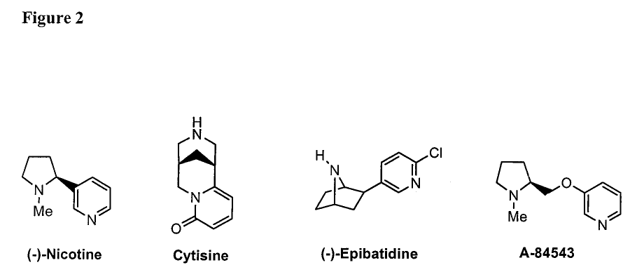

### DETAILED DESCRIPTION OF THE INVENTION

The present invention provides substituted cytisine compounds and pharmaceutical compositions comprising the same. The cytisine compounds of the invention can be used for treating diseases impacted by a nicotinic ACh receptor, such as Alzheimer's disease, Parkinson's disease, Schizophrenia, and tobacco abuse. The present invention also provides methods for modulating a nicotinic ACh receptor in vivo or in vitro. Although cytisine has been reported to bind to nicotinic ACh receptors, the Applicants have surprisingly discovered that substitution at the 10-position of cytisine provides compounds that bind with high selectivity to various nicotinic ACh receptor subtypes. Binding selectivity is important for therapeutic applications because different receptor subtypes have unique pharmacological and biophysical properties. Thus, compounds that bind to a nicotinic ACh receptor with high selectivity may provide more efficacious treatments with reduced side effects.

Neuronal nicotinic acetylcholine receptors (nAChRs) are differently expressed in many regions of the central and peripheral nervous system.^{1,2} These receptors are made up of various combinations of subunits. At present, seventeen (α1-α10, β1-β4, γ, δ and ε) different types of subunits that have been identified. The different receptor subtypes are found at different locations within the nervous system and have important implications for therapeutic treatments because various subunits have unique pharmacological and biophysical properties.^{3,4} The α4β2 nAChR is the most abundant subtype in the brain.⁷ Several findings suggest that α4β2 receptors are involved in behavioral activity, such as nicotine dependence, avoidance learning, and antinociception.⁸

Nicotine (1) and epibatidine (2) are both naturally occurring nAChR agonists that have attracted interest as lead candidates for analog synthesis aimed at identifying structures with improved pharmacological properties.^{1,9,10} For example, we recently reported that introduction of a hydrophobic or hydrogen-bonding alkynyl group into the C-5 position of the pyridine ring of epibatidine and A-84543 (3) significantly increased the selectivity for nAChRs containing β2 subunits.¹¹

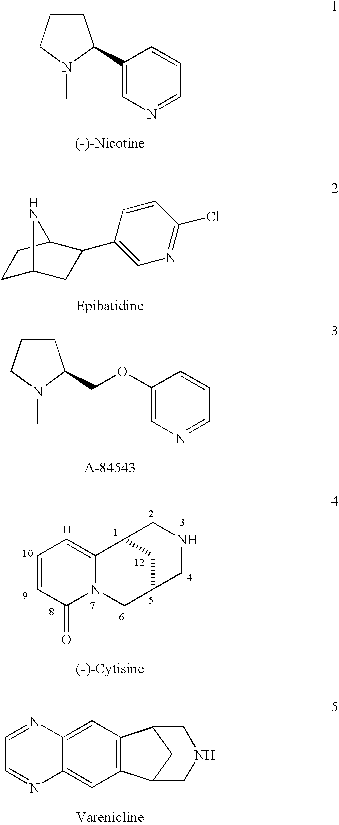

(−)-Cytisine (4) is a natural quinolizidine alkaloid reported to behave as a partial agonist at the α4β2 nAChR with EC₅₀≈1 μM having nanomolar binding affinity (Kᵢ≈1 nM).¹²⁻¹⁶ [³H]Cytisine has been used as a radioligand in the study of nAChRs.^{13,17} Three total syntheses of cytisine¹⁸ were achieved in the 1950s. Recently, further interest in this alkaloid was stimulated by the two alternative approaches to cytisine reported by Coe¹⁹ and O'Neill et al.²⁰ Their efforts resulted in the discovery of varenicline (5), a substantially re-engineered version of cytisine which has become a clinical candidate for use in smoking cessation.²¹ Several other reports, including two enantioselective routes to this alkaloid, have been published along with reports of certain cytisine analogs. ²²⁻²⁴

Herein, we describe the synthesis and pharmacological evaluation of certain 10-substituted cytisine compounds of the invention. Procedures for the preparation of the 10-substituted cytisine compounds were based on O'Neill's strategy, except modified so as to introduce the desired structural changes. ²⁰ At the onset of our work in this area, we chose to synthesize some simplified cytisine analogs. Deletion of the C-1/C-13 bond in cytisine yields structure 6. This compound, and its isomers 7a and 7b, can be prepared from piperidin-3-yl- and -4-ylmethanol by N-protection, iodide installation, and then reaction with α-pyridone, followed by deprotection. Since initial biological assays indicated that the binding affinities of compounds 6a-c and 7a-b at the nAChR subtypes were lower than that of cytisine,¹⁶ we subsequently retained the core tricyclic structure while placing substituents on the pyridone ring.

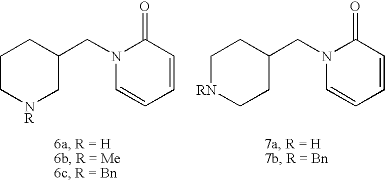

In order to prepare 10-hydroxymethyl cytisine, we first prepared compound 10 by borane reduction of commercially available 2-chloro-6-methoxyisonicotinic acid (8),²⁵ followed by hydroxyl protection (Scheme 1).

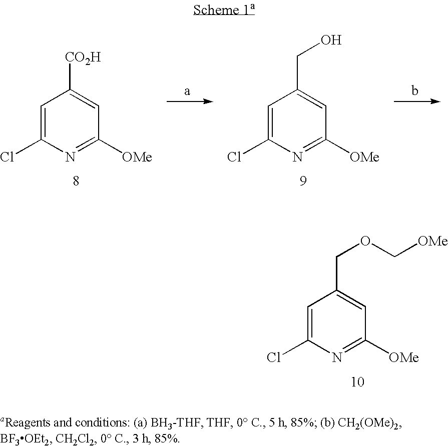

Pd-catalyzed Stille coupling of preformed stannane²⁰ 11 with 10 under the optimized conditions proceeded smoothly to afford compound 12 in 79% isolated yield (Scheme 2). With trans-benzyl(chloro)bis(triphenylphosphine)palladium(II) as the catalyst, the reaction proceeded much faster, but gave low yields on scale-up. The required alcohol was obtained using 1 M LiAlH₄ solution in THF at −20° C. for 3.5 h. Following the strategy of O'Neill with some modifications, and after deprotection of the methoxymethyl (MOM) group with trifluoroacetic acid (TFA) at room temperature, we obtained N-benzyl-10-(hydroxymethyl) cytisine (14).

Debenzylation of 14 using 0.01 eq. of 20% Pd(OH)₂—C with H₂ (1 atm) in the presence of (Boc)₂O and MeOH for 5 minutes at reflux gave 10-hydroxymethyl analog 17 in 97% yield. Hydrogenation of N-benzylcytisine 14 over Pd—C in the presence of Boc₂O provided 16 and 17 as a mixture of products.²⁷ Semi-preparative HPLC purification of the resultant mixture gave the less polar compound 16 and the more polar 10-hydroxymethyl derivative 17. Final N-Boc deprotection with TFA²⁸ gave 10-substituted racemic cytisine derivatives 15 and 17a.

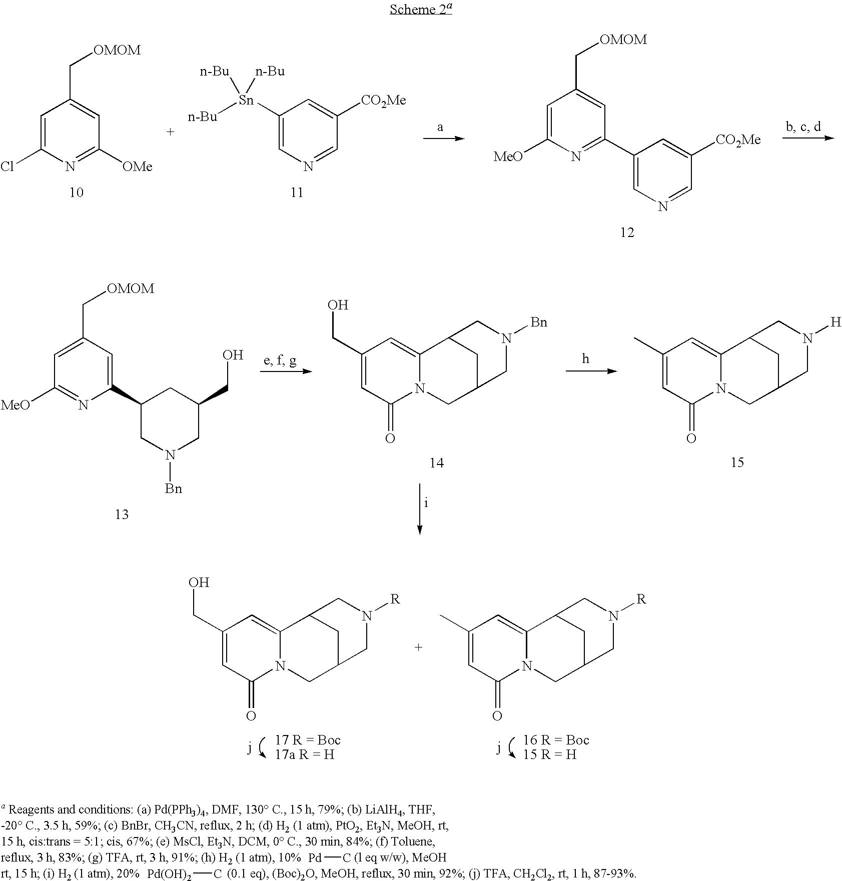

We further expanded the SAR of cytisine by preparing some additional analogs starting from (−)-cytisine itself Most of the previously reported SARs of this molecule have focused on modifications at the alicyclic nitrogen (position 3) and also on the 9- and 11-positions of the pyridone ring.^{24,29,30} Moreover, a recent report showed that substitution at the 6-position could be brought about via a novel N-acyl migration reaction.³¹ As the biological activity of some of these compounds has not been described in full, we selected four of the compounds together with new analogs to extend the SAR studies. Following a literature procedure we synthesized the N-Boc-protected 9-bromocytisine 18 and its Stille coupling product with tri-n-butylvinylstannane.²⁹ Final deprotection with TFA afforded the derivative (−)-19 (Scheme 3).^{29b}

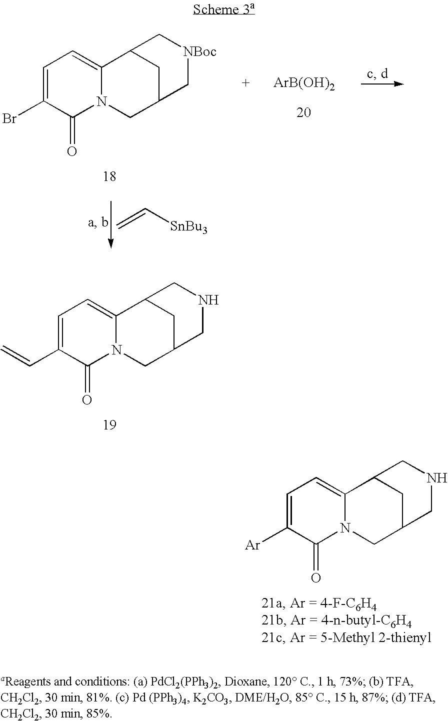

Suzuki coupling of 18 with various boronic acids 20 gave 21a-c (Scheme 3). The synthesis of 21a using the Stille coupling procedure²⁹ has already been reported. The 6-substituted derivatives 22 and 23 were also prepared following known procedures.³¹

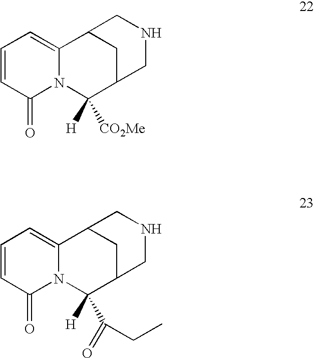

Other compounds amendable to the present invention include compounds 24-29 depicted below. As illustrated below, the C-10 position of cytisine can be substituted with a variety of alkyl, cycloalkyl, and aryl groups by way of a heteroalkyl linker.

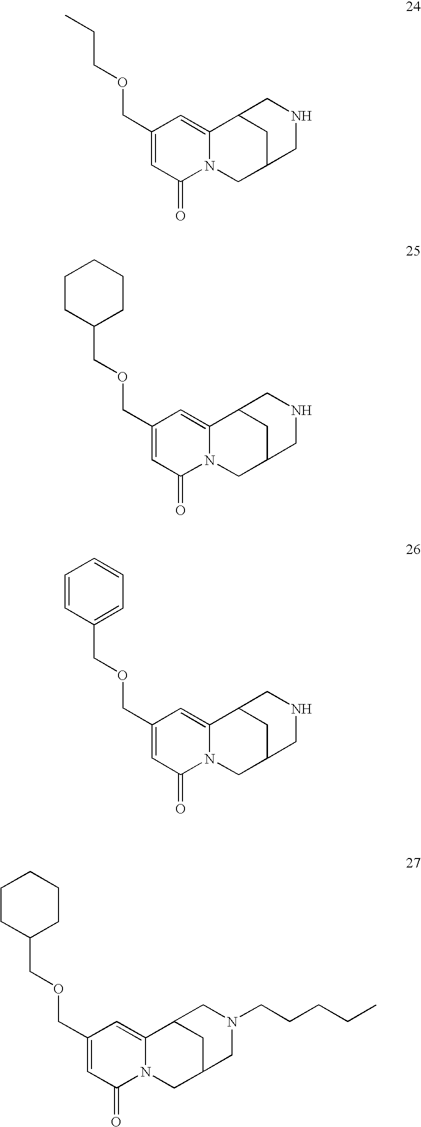

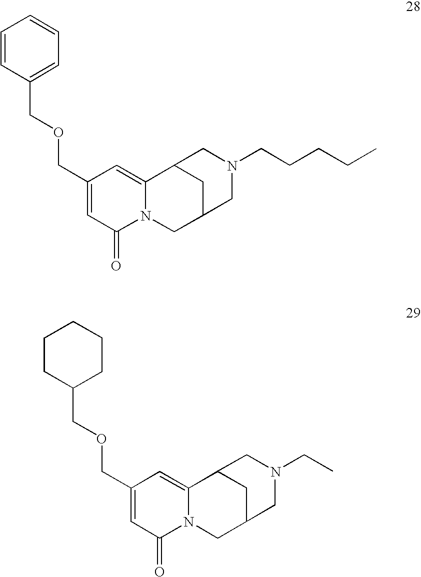

The in vitro binding affinity (Kᵢ value) of 10 cytisine analogs (15, 17a, 19, 21a, 21b, 21c, 22-25) was measured at six defined nAChR subtypes expressed in stably transfected cell lines using competition binding assays as previously reported (Table 1).^{10,16,32} Surprisingly, compound 15 showed high selectivity for the α4β2 subtype over the other subtypes. This is especially true for the selectivity between the α4β2 subtype and α3β4 subtype, where the affinity ratio of α3β4/α4β2 is larger than 3000-fold. Notably, the α4β2 subtype is the main subtype of ganglionic nAChRs. Compound 17a, with a 10-hydroxymethyl group, also has a larger α3β4/α4β2 affinity ratio than cytisine. The 9-vinyl compound 19 was slightly more potent than cytisine at some of the nAChRs.

TABLE 1
Binding affinities and calculated lipophilicities of (±)-Epibatidine (2), (−)-Cytisine
(4) and Cytisine analogs at nAChR subtypes.^{a}

| | | | | | | | | Affinity | |
|---|---|---|---|---|---|---|---|---|---|
| | Kᵢ (nM)^{b} | | | | | | | ratio | Calculated |
| Ligand | α2β2 | α2β4 | α3β2 | α3β4 | α4β2 | α4β4 | Forebrain | (α3β4/α4β2) | ClogP^{c} |
| rac-15 | 7.5 | 180 | 540 | 6700 | 1.9 | 38 | 20 | 3526 | 1.15 |
| rac-17a | 32 | 300 | 467 | 10000 | 11 | 68 | 38 | 909 | −0.32 |
| (−)-19 | 0.7 | 9.0 | 28 | 95 | 0.73 | 2.3 | 5.2 | 130 | 1.50 |
| (−)-21a | 820 | 8100 | 12000 | 66000 | 420 | 3300 | 3100 | 157 | 2.56 |
| (−)-21b | 8000 | 28000 | 36000 | 140000 | 8200 | 13000 | 21000 | 17 | 4.38 |
| (−)-21c | 500 | 1700 | 6000 | 23000 | 390 | 590 | 1200 | 59 | 2.73 |
| (−)-22 | 17000 | 75000 | 250000 | 320000 | 24000 | 33000 | 48000 | 13 | 0.57 |
| (−)-23 | 14000 | 50000 | 300000 | 280000 | 16000 | 20000 | 23000 | 18 | 0.71 |
| rac-24^{d} | 31 | 180 | 1300 | 5100 | 130 | 32 | 99 | 39 | 1.36 |
| rac-25^{e} | <17 | 780 | 800 | 25000 | <29 | 150 | 170 | >862 | 3.14 |
| (±)-Epibatidine | 0.02 | 0.09 | 0.04 | 0.57 | 0.06 | 0.16 | 0.06 | 10 | 1.81 |
| (2) | | | | | | | | | |
| (−)-Cytisine (4) | 1.07 | 5.41 | 37.20 | 217.00 | 1.51 | 2.10 | 1.92 | 144 | 0.60 |

^{a}Competition binding assays were carried out in membrane homogenates of stably transfected cells or rat forebrain tissue as described previously.¹⁶ The nAChRs were labeled with [³H]epibatidine. The K_{d} values for [³H]epibatidine used for calculating Kᵢ values were 0.02 for α2β2, 0.08 for α2β4, 0.03 for α3β2, 0.3 for α3β4, 0.04 for α4β2, 0.09 for α4β4 and 0.05 for rat forebrain.
^{b}Kᵢ values of the cytisine analogs shown are the mean of three to five independent measurements. For clarity, the SEM for the Kᵢ values shown are omitted, but in all cases were less than 45% of the mean values. The Kᵢ values of epibatidine (2) and cytisine (4) were published previously and are shown here for comparison.¹⁶
^{c}The ClogP values are calculated using the online version of Syracuse.
^{d}Competition binding with [3H]-Epibatidine, concentration range 0.0000381-10 μM.
^{e}Estimated Kᵢ (nM) from single run concentration binding assay.

TABLE 2
Comparison of agonist activities of(−)-nicotine and (−)-19 at two major
nAChR subtypes, α3β4 and α4β2.

| | α3β4 nAChR's^{b} | | α4β2 nAChR's^{c} | |
|---|---|---|---|---|
| | | Relative Eₘₐₓ | | Relative Eₘₐₓ |
| | EC₅₀ | (% of | EC₅₀ | (% of |
| Compound | (μM) | nicotine Eₘₐₓ) | (μM) | nicotine Eₘₐₓ) |
| (−)-Nicotine | 35 ± 8 | 100 | 10 ± 1 | 100 |
| (−)-19 | 30 ± 7 | 83 ± 3 | 1.3 ± 0.4 | 22 ± 2 |

^{a}Agonist activities were measured using ⁸⁶ Rb⁺ efflux assays. Values shown are the mean ± standard error of three independent experiments performed in quadruplicate.
^{b}KXα3β4R2 cells stably expressing rat α3β4 nAChRs were used as described previously.^{16,32,33}

The above eight cytisine analogs were next tested for their agonist activities at the two major neuronal nAChR subtypes, α3β4 and α4β2 using ⁸⁶Rb⁺ efflux assays previously reported. ^{15,32} They were tested at 4 concentrations (0.1, 1, 10 and 100 μM). Compound 19 stimulated ⁸⁶Rb⁺ efflux from cells expressing either α3β4 or α4β2 nAChR subtypes. Compound 19 was further evaluated for its agonist potency and efficacy (Table 2). Consistent with its higher binding affinity at α4β2 than at α3β4 nAChRs, the compound was 20-fold more potent at the α4β2 subtype (EC₅₀=1.3 μM) than at the α3β4 subtype (EC₅₀=30 μM). Compared to the efficacy of (−)-nicotine, the maximal efficacies of 19 were 83% and 22% of those of nicotine at the α3β4 receptors and α4β2 receptors, respectively. Certain aspects of the agonist activity profile of 19 mirror that of cytisine.^{15,33} The other seven compounds did not show agonist activity at the concentrations used at these two nAChR subtypes.

Compounds 15 and 17a are antagonists of the α4β2 nAChR subtype. We investigated the antagonist properties of compounds 15 and 17a because they did not show agonist activity at α4β2 nAChRs despite their high selectivity for this nAChR subtype in the binding assays. Compounds 15 and 17a were tested for their antagonist activities at the α4β2 and α3β4 receptors at concentrations from 0.1 μM to 100 μM. The test results indicate that the compounds did not significantly block nicotine stimulated responses at concentrations up to 10 μM. However, at 100 μM, both compounds inhibited more than 50% of the function of the α4β2 nAChR subtype but only slightly inhibited the function of the α3β4 nAChR subtype. Thus, compounds 15 and 17a appear to have high affinity for the α4β2 nAChR subtype in its desensitized conformation (i.e., in the receptor binding assays), but low affinity for the receptors in their resting conformation, as shown by their low potency in functional assays. This is typical of most classical nicotinic ligands.³⁴

While (−)-cytisine is a potent, partial α4β2 nAChR agonist, it does not show strong efficacy as a smoking cessation aid.³⁵ This lack of efficacy may result at least in part from its poor penetration of the blood-brain barrier (BBB).³⁶ Lipophilicity is an important indicator for predicting absorption and BBB penetration.³⁷ Compound 19 has a higher calculated ClogP value than that of cytisine (Table 1).³⁸ Its ClogP value is between that of nicotine (1.00) and epibatidine (1.80), both of which penetrate the BBB easily. This data suggests that compound 19 may have an improved BBB penetration in comparison to cytisine. Accordingly, compound 19 may be a better candidate to use in targeting CNS receptors in vivo, in particular for use in smoking cessation. The calculated ClogP value for compounds 26-29 is presented in Table 3.

TABLE 3
Calculated lipophilicities of cytisine analogs 26-29.

| Compound | Calculated ClogP |
|---|---|
| 26 | 2.09 |
| 27 | 5.32 |
| 28 | 4.26 |
| 29 | 3.84 |

### REFERENCES

(1) Holladay, M. W.; Dart, M. J.; Lynch, J. K. Neuronal nicotinic acetylcholine receptors as targets for drug discovery. J. Med. Chem. 1997, 40, 4169-4194.
(2) (a) Karlin, A. Emerging structure of the nicotinic acetylcholine receptors. Nat. Rev. Neurosci. 2002, 3, 102-114; (b) Jensen, A. A.; Frølund, B.; Liljefors, T.; Krogsgaard-Larsen, P. Neuronal nicotinic acetylcholine receptors: structural revelations, target identifications, and therapeutic inspirations. J. Med. Chem. 2005, 48, 4705-4745.
(3) (a) Hogg, R. C.; Bertrand, D. Nicotinic acetylcholine receptors as drug targets. Curr. Drug. Targets. CNS Neurol. Disord. 2004, 3, 123-130; (b) Clementi, F.; Fornasari, D.; Gotti, C. Neuronal nicotinic acetylcholine receptors: from structure to therapeutics. Trends Pharmacol Sci 2000, 21, 35-37; (c) Kellar, K. J.; Dâavila-Garcâia, M. I.; Xiao, Y. Pharmacology of neuronal nicotinic acetylcholine receptors: effects of acute and chronic nicotine. Nicotine Tob. Res. 1999, 1, S117-120; discussion S139-140; (d) Daly, J. W. Nicotinic Agonists, Antagonists and Modulators From Natural Sources. Cell. Mol. Neurobiol. 2005, 25, 513-551.
(4) Millar, N. S. Assembly and subunit diversity of nicotinic acetylcholine receptors. Biochem. Soc. Trans. 2003, 31, 869-874.
(5) Gotti, C.; Clementi, F. Neuronal nicotinic receptors: from structure to pathology. Prog. Neurobiol. 2004, 74, 363-396.
(6) Lloyd, G. K.; Williams, M. Neuronal nicotinic acetylcholine receptors as novel drug targets. J Pharmacol. Exp. Ther. 2000, 292, 461-467.
(7) Flores, C. M.; Rogers, S. W.; Pabreza, L. A.; Wolfe, B. B.; Kellar, K. J. A subtype of nicotinic cholinergic receptor in rat brain is composed of alpha 4 and beta 2 subunits and is up-regulated by chronic nicotine treatment. Mol. Pharmacol. 1992, 41, 31-37.
(8) (a) Cordero-Erausquin, M.; Marubio, L. M.; Klink, R.; Changeux, J. P. Nicotinic receptor function: new perspectives from knockout mice. Trends Pharmacol. Sci. 2000, 21, 211-217; (b) Picciotto, M. R.; Zoli, M.; Rimondini, R.; Lâena, C.; Marubio, L. M.; Pich, E. M.; Fuxe, K.; Changeux, J. P. Acetylcholine receptors containing the beta2 subunit are involved in the reinforcing properties of nicotine. Nature 1998, 391, 173-177; (c) Marubio, L. M.; Gardier, A. M.; Durier, S.; David, D.; Klink, R.; Arroyo-Jimenez, M. M.; McIntosh, J. M.; Rossi, F.; Champtiaux, N.; Zoli, M.; Changeux, J. P. Effects of nicotine in the dopaminergic system of mice lacking the alpha4 subunit of neuronal nicotinic acetylcholine receptors. Eur. J. Neurosci. 2003, 17, 1329-1337; (d) Tapper, A. R.; McKinney, S. L.; Nashmi, R.; Schwarz, J.; Deshpande, P.; Labarca, C.; Whiteaker, P.; Marks, M. J.; Collins, A. C.; Lester, H. A. Nicotine activation of alpha4* receptors: sufficient for reward, tolerance, and sensitization. Science 2004, 306, 1029-1032.
(9) (a) Ferretti, G.; Dukat, M.; Giannella, M.; Piergentili, A.; Pigini, M.; Quaglia, W.; Damaj, M. I.; Martin, B. R.; Glennon, R. A. Binding of nicotine and homoazanicotine analogues at neuronal nicotinic acetylcholinergic (nACh) receptors. Bioorg. Med. Chem. Lett. 2003, 13, 733-735; (b) Tonder, J. E.; Olesen, P. H. Agonists at the alpha4beta2 nicotinic acetylcholine receptors: structure-activity relationships and molecular modelling. Curr. Med. Chem. 2001, 8, 651-674; (c) Carroll, F. I.; Lee, J. R.; Navarro, H. A.; Ma, W.; Brieaddy, L. E.; Abraham, P.; Damaj, M. I.; Martin, B. R. Synthesis, nicotinic acetylcholine receptor binding, and antinociceptive properties of 2-exo-2-(2′,3′-disubstituted 5′-pyridinyl)-7-azabicyclo[2.2.1]heptanes: epibatidine analogues. J. Med. Chem. 2002, 45, 4755-4761; (d) Wei, Z. L.; Xiao, Y.; Kellar, K. J.; Kozikowski, A. P. Synthesis and pharmacological characterization of bivalent ligands of epibatidine at neuronal nicotinic acetylcholine receptors. Bioorg. Med. Chem. Lett. 2004, 14, 1855-1858.
(10) Wei, Z. L.; Xiao, Y.; George, C.; Kellar, K. J.; Kozikowski, A. P. Functionalization of the alicyclic skeleton of epibatidine: synthesis and nicotinic acetylcholine receptor binding affinities of epibatidine analogues. Org. Biomol. Chem. 2003, 1, 3878-3881.
(11) Wei, Z. L.; Xiao, Y.; Yuan, H.; Baydyuk, M.; Petukhov, P. A.; Musachio, J. L.; Kellar, K. J.; Kozikowski, A. P. Novel pyridyl ring C5 substituted analogues of epibatidine and 3-(1-methyl-2(S)-pyrrolidinylmethoxy)pyridine (A-84543) as highly selective agents for neuronal nicotinic acetylcholine receptors containing beta2 subunits. J. Med. Chem. 2005, 48, 1721-1724.
(12) Ing, H. R. Structure determination. J. Chem. Soc. 1932, 2778.
(13) Pabreza, L. A.; Dhawan, S.; Kellar, K. J. [³H]cytisine binding to nicotinic cholinergic receptors in brain. Mol. Pharmacol. 1991, 39, 9-12.
(14) (a) Hall, M.; Zerbe, L.; Leonard, S.; Freedman, R. Characterization of [³H]cytisine binding to human brain membrane preparations. Brain Res. 1993, 600, 127-133; (b) Anderson, D. J.; Arneric, S. P. Nicotinic receptor binding of [³H]cytisine, [³H]nicotine and [³H]methylcarbamylcholine in rat brain. Eur. J. Pharmacol. 1994, 253, 261-267.
(15) Eaton, J. B.; Peng, J. H.; Schroeder, K. M.; George, A. A.; Fryer, J. D.; Krishnan, C.; Buhlman, L.; Kuo, Y. P.; Steinlein, O.; Lukas, R. J. Characterization of human alpha 4 beta 2-nicotinic acetylcholine receptors stably and heterologously expressed in native nicotinic receptor-null SH-EP1 human epithelial cells. Mol. Pharmacol. 2003, 64, 1283-1294.
(16) Xiao, Y.; Kellar, K. J. The comparative pharmacology and up-regulation of rat neuronal nicotinic receptor subtype binding sites stably expressed in transfected mammalian cells. J. Pharmacol. Exp. Ther. 2004, 310, 98-107.
(17) Flesher, J. E.; Scheffel, U.; London, E. D.; Frost, J. J. In vivo labeling of nicotinic cholinergic receptors in brain with [³H]cytisine. Life. Sci. 1994, 54, 1883-1890.
(18) (a) Govindachari, T. R.; Rajadurai, S.; Subramanian, M.; Thyagarajan, B. S. J. Chem. Soc. 1957, 3839-3844; (b) Van Tamelen, E. E.; Baran, J. S. The synthesis of dl-cytisine. J. Am. Chem. Soc. 1955, 77, 4944-4945; (b) Van Tamelen, E. E.; Baran, J. S. Total synthesis of oxygenated lupin alkaloids. J. Am. Chem. Soc. 1958, 80, 4659-4670.
(19) Coe, J. W. Total synthesis of (+/−)-cytisine via the intramolecular Heck cyclization of activated N-alkyl glutarimides. Org. Lett. 2000, 2, 4205-4208.
(20) O'Neill, B. T.; Yohannes, D.; Bundesmann, M. W.; Arnold, E. P. Total synthesis of (+/−)-cytisine. Org. Lett. 2000, 2, 4201-4204.
(21) Coe, J. W.; Brooks, P. R.; Vetelino, M. G.; Wirtz, M. C.; Arnold, E. P.; Huang, J.; Sands, S. B.; Davis, T. I.; Lebel, L. A.; Fox, C. B.; Shrikhande, A.; Heym, J. H.; Schaeffer, E.; Rollema, H.; Lu, Y.; Mansbach, R. S.; Chambers, L. K.; Rovetti, C. C.; Schulz, D. W.; Tingley, F. D., III; O'Neill, B. T. Varenicline: an a4b2 nicotinic receptor partial agonist for smoking cessation. J. Med. Chem. 2005, 48, 3474-3477.
(22) (a) Botuha, C.; Galley, C. M.; Gallagher, T. A short synthesis of (+/−)-cytisine. Org. Biomol. Chem. 2004, 2, 1825-1826; (b) Danieli, B.; Lesma, G.; Passarella, D.; Sacchetti, A.; Silvani, A.; Virdis, A. Total enantioselective synthesis of (−)-cytisine. Org. Lett. 2004, 6, 493-496; (c) Nshimyumukiza, P. C., D.; Rouden, J.; Lasne, M. C.; Plaquevent, J. C. Construction of functionalized/substituted bipyridines by means of negishi cross-coupling reactions. Formal synthesis of (+−)-cytisine. Tetrahedron Lett. 2001, 42, 7787-7790; (d) Coe, J. W.; Vetelino, M. G.; Bashore, C. G.; Wirtz, M. C.; Brooks, P. R.; Arnold, E. P.; Lebel, L. A.; Fox, C. B.; Sands, S. B.; Davis, T. I.; Schulz, D. W.; Rollema, H.; Tingley, F. D., 3rd; O'Neill, B. T. In pursuit of alpha4beta2 nicotinic receptor partial agonists for smoking cessation: carbon analogs of (−)-cytisine. Bioorg. Med. Chem. Lett. 2005, 15, 2974-2979; (e) Stead, D.; O'Brien, P.; Sanderson, A. J. Concise synthesis of (+/−)-cytisine via lithiation of N-Boc-bispidine. Org. Lett. 2005, 7, 4459-4462.
(23) Honda, T.; Takahashi, R.; Namiki, H. Syntheses of (+)-cytisine, (−)-kuraramine, (−)-isokuraramine, and (−)-jussiaeiine A. J. Org. Chem. 2005, 70, 499-504.
(24) Fitch, R. W.; Kaneko, Y.; Klaperski, P.; Daly, J. W.; Seitz, G.; Gèundisch, D. Halogenated and isosteric cytisine derivatives with increased affinity and functional activity at nicotinic acetylcholine receptors. Bioorg. Med. Chem. Lett. 2005, 15, 1221-1224.
(25) Adamczyk, M. A., S. R.; Reddy, R. E. Nonproteinogenic amino acids: an efficient asymmetric synthesis of (S)-(−)-acromelobic acid and (S)-(−)-acromelobinic acid. Tetrahedron 2002, 58, 6951-6963.
(26) Pichon, N.; Harrison-Marchand, A.; Mailliet, P.; Maddaluno, J. Diastereoselective syntheses of new analogues of the farnesyltransferase inhibitor RPR 130401. J. Org. Chem. 2004, 69, 7220-7227.
(27) Gillaizeau-Gauthier, I.; Royer, J.; Husson, H.-P. Toward an asymmetric general access to azabicyclo[n.2.1]alkanes according to the CN(R,S) method. Eur. J. Org. Chem. 2002, 1484-1489.
(28) Roger, G.; Lagnel, B.; Rouden, J.; Besret, L.; Valette, H.; Demphel, S.; Gopisetti, J.; Coulon, C.; Ottaviani, M.; Wrenn, L. A.; Letchworth, S. R.; Bohme, G. A.; Benavides, J.; Lasne, M. C.; Bottlaender, M.; Dollâe, F. Synthesis of a [2-pyridinyl-¹⁸F]-labelled fluoro derivative of (−)-cytisine as a candidate radioligand for brain nicotinic alpha4beta2 receptor imaging with PET. Bioorg. Med. Chem. 2003, 11, 5333-5343.
(29) (a) Marriáere, E.; Rouden, J.; Tadino, V.; Lasne, M. C. Synthesis of analogues of (−)-cytisine for in vivo studies of nicotinic receptors using positron emission tomography. Org. Lett. 2000, 2, 1121-1124; (b) O'Neill, B. T. PCT Int. Appl. WO9818798, 1998: Abstr. 1998, 129, 4774k.
(30) (a) Canu Boido, C.; Carotti, A; Sparatore, F.; Cytisine derivatives as ligands for neuronal nicotine receptors and with various pharmacological activities. Il Farmaco. 2003, 58, 265-277; (b) Nicolotti, O.; Canu Boido, C.; Sparatore, F.; Carotti, A. Cytisine derivatives as high affinity nAChR ligands: synthesis and comparative molecular field analysis. Il Farmaco 2002, 57, 469-478.
(31) Rouden, J.; Ragot, A.; Gouault, S.; Cahard, D.; Plaquevent, J. C.; Lasne, M. C. Regio- and diastereoselective functionalization of (−)-cytisine: an unusual N—C acyl migration. Tetrahedron: Asymmetry 2002, 13, 1299-1305.
(32) Xiao, Y.; Meyer, E. L.; Thompson, J. M.; Surin, A.; Wroblewski, J.; Kellar, K. J. Rat alpha3/beta4 subtype of neuronal nicotinic acetylcholine receptor stably expressed in a transfected cell line: pharmacology of ligand binding and function. Mol. Pharmacol. 1998, 54, 322-333.
(33) Meyer, E. L.; Xiao, Y.; Kellar, K. J. Agonist regulation of rat alpha 3 beta 4 nicotinic acetylcholine receptors stably expressed in human embryonic kidney 293 cells. Mol. Pharmacol. 2001, 60, 568-576.
(34) Quick, M. W.; Lester, R. A. Desensitization of neuronal nicotinic receptors. J. Neurobiol. 2002, 53, 457-478.
(35) Scharfenberg, G.; Benndorf, S.; Kempe, G. [Cytisine (Tabex) as a pharmaceutical aid in stopping smoking]. Dtsch. Gesundheitsw. 1971, 26, 463-465.
(36) (a) Barlow, R. B.; McLeod, L. J. Some studies on cytisine and its methylated derivatives. Br. J. Pharmacol. 1969, 35, 161-174; (b) Reavill, C.; Walther, B.; Stolerman, I. P.; Testa, B. Behavioural and pharmacokinetic studies on nicotine, cytisine and lobeline. Neuropharmacology 1990, 29, 619-624; (c) Romano, C.; Goldstein, A.; Jewell, N. P. Characterization of the receptor mediating the nicotine discriminative stimulus. Psychopharmacology (Berl) 1981, 74, 310-315.
(37) (a) Waterhouse, R. N. Determination of lipophilicity and its use as a predictor of blood-brain barrier penetration of molecular imaging agents. Mol. Imaging. Biol. 2003, 5, 376-389; (b) Laruelle, M.; Slifstein, M.; Huang, Y. Relationships between radiotracer properties and image quality in molecular imaging of the brain with positron emission tomography. Mol. Imaging. Biol. 2003, 5, 363-375; (c) Smith, D. A.; Jones, B. C.; Walker, D. K. Design of drugs involving the concepts and theories of drug metabolism and pharmacokinetics. Med. Res. Rev. 1996, 16, 243-266.
(38) Meylan, W. M.; Howard, P. H. Atom/fragment contribution method for estimating octanol-water partition coefficients. J. Pharm. Sci. 1995, 84, 83-92.

### Binding Affinity Assays

In addition to the binding affinity assays described above and the assays described in the examples, additional assays are provided herein for testing the activity of the compounds of the invention.

Nicotinic ACh receptors in the brain are pentameric structures composed of subunits distinct from those found in skeletal muscles. The existence of eight α-subunits (α2-α9) and three β-subunits (β2-β4) in the mammalian brain has been described. The predominant subtype with high affinity for nicotine is comprised of three α-subunits and two β-subunits.

The affinity of compounds of the invention for nicotinic ACh receptors may be investigated in three tests for in vitro inhibition of ³H-epibatidin binding, ³H-α-bungarotoxin binding and ³H-cytisine binding as described below.

### In Vitro Inhibition of ³H-cytisine Binding

The predominant subtype with high affinity for nicotine is comprised of α4 and β2 subunits. nAChRs of the latter type may selectively be labelled by the nicotine agonist ³H-cytisine.

Tissue Preparation: Preparations may be performed at 0-4° C. unless otherwise indicated. Cerebral cortices from male Wistar rats (150-250 g) may be homogenized for 20 sec in 15 mL Tris, HCl (50 mM, pH 7.4) containing 120 mM NaCl, 5 mM KCl, 1 mM MgCl₂ and 2.5 mM CaCl₂ using an Ultra-Turrax homogenizer. The homogenate may then be centrifuged at 27,000×g for 10 min. The supernatant may then be discarded and the pellet resuspended in fresh buffer and centrifuged a second time. The final pellet may be resuspended in fresh buffer (35 mL per g of original tissue) and used for binding assays.

Assay: Aliquots of 500 μl homogenate may be added to 25 μl of test solution and 25 μl of ³H-cytisine (1 nM, final concentration), mixed and incubated for 90 min at 2° C. Non-specific binding may then be determined using (−)-nicotine (100 μM, final concentration). After incubation the samples may be added to 5 mL of ice-cold buffer and poured directly onto Whatman GF/C glass fiber filters under suction and immediately washed with 2×5 mL ice-cold buffer. The amount of radioactivity on the filters may then be determined by conventional liquid scintillation counting. Specific binding is total binding minus non-specific binding.

In Vitro Inhibition of ³H-α-bungarotoxin Binding Rat Brain

α-Bungarotoxin is a peptide isolated from the venom of the Elapidae snake Bungarus multicinctus (Mebs et al., Biochem. Biophys. Res. Commun., 44(3), 711 (1971)) and has high affinity for neuronal and neuromuscular nicotinic receptors, where it acts as a potent antagonist. ³H-α-Bungarotoxin binds to a single site in rat brain with a unique distribution pattern in rat brain (Clarke et al., J. Neurosci. 5, 1307-1315 (1985)).

³H-α-Bungarotoxin labels nAChR are formed by the α7 subunit isoform found in the brain and the isoform in the neuromuscular junction (Changeaux, Fidia Res. Found. Neurosci. Found. Lect. 4, 21-168 (1990). Functionally, the α7 homo-oligomer expressed in oocytes has a calcium permeability greater than neuromuscular receptors and, in some instances greater than NMDA channels (Seguela et al., J. Neurosci. 13, 596-604 (1993).

Tissue Preparation: Preparations may be performed at 0-4° C. unless otherwise indicated. Cerebral cortices from male Wistar rats (150-250 g) may be homogenized for 10 sec in 15 mL 20 mM Hepes buffer containing 118 mM NaCl, 4.8 mM KCl, 1.2 mM MgSO₄ and 2.5 mM CaCl₂ (pH 7.5) using an Ultra-Turrax homogenizer. The tissue suspension may then be centrifuged at 27,000×g for 10 min. The supernatant is discarded and the pellet is washed twice by centrifugation at 27,000×g for 10 min in 20 mL fresh buffer, and the final pellet may be resuspended in fresh buffer containing 0.01% BSA (35 mL per g of original tissue) and used for binding assays.

Assay: Aliquots of 500 μl homogenate may be added to 25 μl of test solution and 25 μl of ³H-α-bungarotoxin (2 nM, final concentration), mixed and incubated for 2 h at 37° C. Non-specific binding may then be determined using (−)-nicotine (1 mM, final concentration). After incubation the samples may be added to 5 mL of ice-cold Hepes buffer containing 0.05% PEI and poured directly onto Whatman GF/C glass fibre filters (presoaked in 0.1% PEI for at least 6 h) under suction and immediately washed with 2×5 mL ice-cold buffer. The amount of radioactivity on the filters may then be determined by conventional liquid scintillation counting. Specific binding is total binding minus non-specific binding.

### In Vitro Inhibition of ³H-epibatidin Binding

As discussed previously, Epibatidin is an alkaloid that was first isolated from the skin of the Ecuadorian frog Epipedobates tricolor and was found to have very high affinity for neuronal nicotinic receptors, where it acts as a potent agonist. It is believed that ³H-epibatidin binds to two sites in rat brain, both of which have pharmacological profiles consistent with neuronal nicotinic receptors and a similar brain regional distribution (Hougling et al., Mol. Pharmacol. 48, 280-287 (1995)).

The high affinity binding site for ³H-epibatidin is most certainly binding to the α4β2 subtype of nicotinic receptors. The identity of the low affinity site is still believed to be unknown. The inability of α-bungarotoxin to compete for ³H-epibatidin binding sites may indicate that neither site measured represents the nicotinic receptor composed of α7 subunits.

Tissue preparation: Preparations may be performed at 0-4° C. unless otherwise indicated. The forebrain (cerebellum) from a male Wistar rat (150-250 g) may be homogenized for 10-20 sec in 20 mL Tris, HCl (50 mM, pH 7.4) using an Ultra-Turrax homogenizer. The tissue suspension may then be centrifuged at 27,000×g for 10 min. The supernatant is then discarded and the pellet may then be washed three times by centrifugation at 27,000×g for 10 min in 20 mL fresh buffer, and the final pellet may be resuspended in fresh buffer (400 mL per g of original tissue) and used for binding assays.

Assay: Aliquots of 2.0 mL homogenate may be added to 0.100 mL of test solution and 0.100 mL of ³H-epibatidin (0.3 nM, final concentration), mixed and incubated for 60 min at room temperature. Non-specific binding may then be determined using (−)-nicotine (30 μM, final concentration). After incubation the samples may then be poured directly onto Whatman GF/C glass fibre filters (presoaked in 0.1% PEI for at least 20 min) under suction and immediately washed with 2×5 mL ice-cold buffer. The amount of radioactivity on the filters may be determined by conventional liquid scintillation counting. Specific binding is total binding minus non-specific binding.

### Compounds & Pharmaceutical Compositions of the Invention

One aspect of the invention relates to a compound represented by formula I:

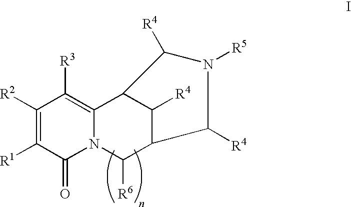

or a pharmaceutically acceptable salt thereof;

wherein

the stereochemical configuration at any stereocenter of said compound is R, S, or a mixture thereof;

R¹, R³, and R⁶ represent independently for each occurrence H, alkyl, cycloalkyl, alkenyl, cycloalkenyl, alkynyl, aryl, aralkyl, halogen, cyano, nitro, —OR⁷, —N(R⁷)₂, —SR⁷, —C(O)R⁷, —CO₂R⁷, —C(O)N(R⁷)₂, —OC(O)R⁷, —N(R⁷)C(O)R⁷, or —(C(R⁸)₂)ₚCR⁸═C(R⁸)₂; wherein said alkyl, cycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷;

R² is alkyl, cycloalkyl, heterocycloalkyl, alkenyl, cycloalkenyl, alkynyl, aryl, aralkyl, halogen, cyano, nitro, —OR⁷, —N(R⁷)₂, —S, —C(O)R⁷, —CO₂R⁷, —C(O)N(R⁷)₂, —OC(O)R⁷, —N(R⁷)C(O)R⁷, —SC(O)R⁷, or —(C(R⁸)₂)ₚCR⁸═C(R⁸)₂; wherein said alkyl, cycloalkyl, heterocycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷; or R² and R³ taken together form a 5-7 member ring containing 0, 1, or 2 heteroatoms selected from the group consisting of O and N; or R¹ and R² taken together form a 5-7 member ring containing 0, 1, or 2 heteroatoms selected from the group consisting of O and N; or R² is

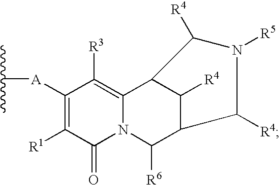

R⁴ represents independently for each occurrence H, alkyl, alkenyl, halogen, —OR⁷, —N(R⁷)₂, or —(C(R⁸)₂)ₚCR⁸═C(R⁸)₂;

R⁵ is H, alkyl, cycloalkyl, alkenyl, cycloalkenyl, alkynyl, aryl, aralkyl, —C(O)R⁷, —CO₂R⁷, —C(O)N(R⁷)₂, or —(C(R⁸)₂)ₚCR⁸═C(R⁸)₂; wherein said alkyl, cycloalkyl, alkenyl, cycloalkenyl, alkynyl, aryl, and aralkyl are optionally substituted with one or more of halogen, —OR⁷, —N(R⁷)₂, —SR⁷, —C(O)R⁷, —CO₂R⁷, —C(O)N(R⁷)₂, —OC(O)R⁷, or —N(R⁷)C(O)R⁷;

R⁷ represents independently for each occurrence H, alkyl, cycloalkyl, heterocycloalkyl, alkenyl, cycloalkenyl, allyl, alkynyl, aryl, aralkyl, alkoxyalkyl, aryloxyalkyl, or cycloalkyloxyalkyl;

R⁸ represents independently for each occurrence H or (C₁-C₆)alkyl;

A is an alkyl diradical, alkenyl diradical, aryl diradical, aralkyl diradical, or —(C(R⁸)₂)ₘ—X—(C(R⁸)₂)ₘ—;

X is O, —N(R⁷)—, or S;

m and p represent independently for each occurrence 1, 2, 3, 4, 5, or 6; and

n is 1 or 2.

In certain embodiments, the present invention relates to the aforementioned compound, wherein R¹ represents independently for each occurrence H, alkyl, alkenyl, aryl, aralkyl, halogen, —C(O)R⁷, —CO₂R⁷, —C(O)N(R⁷)₂, or —(C(R⁸)₂)ₚCR⁸═C(R⁸)₂.

In certain embodiments, the present invention relates to the aforementioned compound, wherein R¹ represents independently for each occurrence H or (C₁-C₆)alkyl.

In certain embodiments, the present invention relates to the aforementioned compound, wherein R¹ represents independently for each occurrence H.

In certain embodiments, the present invention relates to the aforementioned compound, wherein R² is alkyl, cycloalkyl, heterocycloalkyl, alkenyl, cycloalkenyl, alkynyl, aryl, aralkyl, halogen, cyano, nitro, —OR⁷, —N(R⁷)₂, —SR⁷, —C(O)R⁷, —CO₂R⁷, —C(O)N(R⁷)₂, —OC(O)R⁷, —N(R⁷)C(O)R⁷, or —(C(R⁸)₂)ₚCR⁸═C(R⁸)₂; wherein said alkyl, cycloalkyl, heterocycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷.

In certain embodiments, the present invention relates to the aforementioned compound, wherein R² is alkyl, cycloalkyl, heterocycloalkyl, alkenyl, aryl, aralkyl, halogen, cyano, —C(O)R⁷, —CO₂R⁷, —C(O)N(R⁷)₂, or —(C(R⁸)₂)ₚCR⁸═C(R⁸)₂; wherein said alkyl, cycloalkyl, heterocycloalkyl, alkenyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷.

In certain embodiments, the present invention relates to the aforementioned compound, wherein R² is alkyl, cycloalkyl, alkenyl, aryl, or —(C(R⁸)₂)ₚCR⁸═C(R⁸)₂; wherein said alkyl, cycloalkyl, alkenyl, and aryl are optionally substituted with one or more of halogen, —OR⁷, —N(R⁷)₂, or —SR⁷; wherein R⁷ is H or (C₁-C₆)alkyl.

In certain embodiments, the present invention relates to the aforementioned compound, wherein R² is alkyl or cycloalkyl; wherein said alkyl and cycloalkyl are optionally substituted with one or more of halogen, —OR⁷, —N(R⁷)₂, or —SR⁷; wherein R⁷ is H or (C₁-C₆)alkyl.

In certain embodiments, the present invention relates to the aforementioned compound, wherein R² is (C₁-C₆)alkyl optionally substituted with —OR⁷, —N(R⁷)₂, or —SR⁷; wherein R⁷ is H or (C₁-C₆)alkyl.

In certain embodiments, the present invention relates to the aforementioned compound, wherein R² is (C₁-C₆)alkyl optionally substituted with —OR⁷; wherein R⁷ is H or (C₁-C₆)alkyl.

In certain embodiments, the present invention relates to the aforementioned compound, wherein R² is (C₁-C₆)alkyl.

In certain embodiments, the present invention relates to the aforementioned compound, wherein R² is methyl, ethyl, propyl, isopropyl, butyl, isobutyl, sec-butyl, or pentyl optionally substituted with —OR⁷; wherein R⁷ is H or (C₁-C₆)alkyl.

In certain embodiments, the present invention relates to the aforementioned compound, wherein R² is methyl, ethyl, propyl, isopropyl, butyl, isobutyl, sec-butyl, or pentyl.

In certain embodiments, the present invention relates to the aforementioned compound, wherein R² is methyl.

In certain embodiments, the present invention relates to the aforementioned compound, wherein R² is —CH₂OH.

In certain embodiments, the present invention relates to the aforementioned compound, wherein R³ represents independently for each occurrence H, alkyl, alkenyl, aryl, aralkyl, halogen, —C(O)R⁷, —CO₂R⁷, —C(O)N(R⁷)₂, or —(C(R⁸)₂)ₚCR⁸═C(R⁸)₂.

In certain embodiments, the present invention relates to the aforementioned compound, wherein R³ represents independently for each occurrence H or (C₁-C₆)alkyl.

In certain embodiments, the present invention relates to the aforementioned compound, wherein R³ represents independently for each occurrence H.

In certain embodiments, the present invention relates to the aforementioned compound, wherein R⁴ represents independently for each occurrence H or alkyl.

In certain embodiments, the present invention relates to the aforementioned compound, wherein R⁴ is H.

In certain embodiments, the present invention relates to the aforementioned compound, wherein R⁵ is H, alkyl, cycloalkyl, aryl, aralkyl, —C(O)R⁷, —CO₂R⁷, —C(O)N(R⁷)₂, or —(C(R⁸)₂)ₚCR⁸═C(R⁸)₂.

In certain embodiments, the present invention relates to the aforementioned compound, wherein R⁵ is H, alkyl, or benzyl.

In certain embodiments, the present invention relates to the aforementioned compound, wherein R⁵ is H.

In certain embodiments, the present invention relates to the aforementioned compound, wherein R⁶ represents independently for each occurrence H, alkyl, alkenyl, aryl, aralkyl, halogen, —C(O)R⁷, —CO₂R⁷, —C(O)N(R⁷)₂, or —(C(R⁸)₂)ₚCR⁸═C(R⁸)₂.

In certain embodiments, the present invention relates to the aforementioned compound, wherein R⁶ represents independently for each occurrence H or (C₁-C₆)alkyl.

In certain embodiments, the present invention relates to the aforementioned compound, wherein R⁶ is H.

In certain embodiments, the present invention relates to the aforementioned compound, wherein R⁷ represents independently for each occurrence H, alkyl, cycloalkyl, heterocycloalkyl, alkenyl, cycloalkenyl, allyl, alkynyl, aryl, or aralkyl.

In certain embodiments, the present invention relates to the aforementioned compound, wherein R⁷ represents independently for each occurrence H, alkyl, cycloalkyl, aryl, or aralkyl.

In certain embodiments, the present invention relates to the aforementioned compound, wherein R⁷ represents independently for each occurrence H, alkoxymethyl, aryloxymethyl, or cycloalkyloxymethyl.

In certain embodiments, the present invention relates to the aforementioned compound, wherein R⁷ represents independently for each occurrence H or alkyl.

In certain embodiments, the present invention relates to the aforementioned compound, wherein R⁷ is H.

In certain embodiments, the present invention relates to the aforementioned compound, wherein n is 1.

In certain embodiments, the present invention relates to the aforementioned compound, wherein R¹, R³, R⁴, R⁵, and R⁶ represent independently for each occurrence H or alkyl.

In certain embodiments, the present invention relates to the aforementioned compound, wherein R¹, R³, R⁴, R⁵, and R⁶ represent independently for each occurrence H or alkyl; and n is 1.

In certain embodiments, the present invention relates to the aforementioned compound, wherein R¹, R³, R⁴, R⁵, and R⁶ represent independently for each occurrence H or alkyl; n is 1; and R² is alkyl, cycloalkyl, heterocycloalkyl, alkenyl, aryl, aralkyl, halogen, cyano, —C(O)R⁷, —CO₂R⁷, —C(O)N(R⁷)₂, or —(C(R⁸)₂)ₚCR⁸═C(R⁸)₂; wherein said alkyl, cycloalkyl, heterocycloalkyl, alkenyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷.

In certain embodiments, the present invention relates to the aforementioned compound, wherein R¹, R³, R⁴, R⁵, and R⁶ represent independently for each occurrence H or alkyl; n is 1; R² is alkyl, cycloalkyl, alkenyl, aryl, or —(C(R⁸)₂)ₚCR⁸═C(R⁸)₂; wherein said alkyl, cycloalkyl, alkenyl, and aryl are optionally substituted with one or more of halogen, —OR⁷, —N(R⁷)₂, or —SR⁷; and R⁷ is H or (C₁-C₆)alkyl.

In certain embodiments, the present invention relates to the aforementioned compound, wherein R¹, R³, R⁴, R⁵, and R⁶ represent independently for each occurrence H or alkyl; n is 1; R² represents independently for each occurrence (C₁-C₆)alkyl optionally substituted with —OR⁷, —N(R⁷)₂, or —SR⁷; and R⁷ is H or (C₁-C₆)alkyl.

In certain embodiments, the present invention relates to the aforementioned compound, wherein R¹, R³, R⁴, R⁵, and R⁶ represent independently for each occurrence H or alkyl; n is 1; R² represents independently for each occurrence methyl, ethyl, propyl, isopropyl, butyl, isobutyl, sec-butyl, or pentyl optionally substituted with —OR⁷; and R⁷ is H or (C₁-C₆)alkyl.

In certain embodiments, the present invention relates to the aforementioned compound, wherein R¹, R³, R⁴, R⁵, and R⁶ are H; n is 1; R² represents independently for each occurrence methyl, ethyl, propyl, isopropyl, butyl, isobutyl, sec-butyl, or pentyl optionally substituted with —OR⁷; and R⁷ is H or (C₁-C₆)alkyl.

In certain embodiments, the present invention relates to the aforementioned compound, wherein R¹, R³, R⁴, R⁵, and R⁶ are H; n is 1; and R² is methyl.

In certain embodiments, the present invention relates to the aforementioned compound, wherein R¹, R³, R⁴, R⁵, and R⁶ are H; n is 1; and R² is —CH₂OH.

In certain embodiments, the present invention relates to the aforementioned compound, wherein the α3β4/α4β2 nAChR subtype binding affinity ratio for said compound of formula I is greater than about 500:1 in a nAChR binding assay.

In certain embodiments, the present invention relates to the aforementioned compound, wherein the α3β4/α4β2 nAChR subtype binding affinity ratio for said compound of formula I is greater than about 1000:1 in a nAChR binding assay.

In certain embodiments, the present invention relates to the aforementioned compound, wherein the α3β4/α4β2 nAChR subtype binding affinity ratio for said compound of formula I is greater than about 2000:1 in a nAChR binding assay.

In certain embodiments, the present invention relates to the aforementioned compound, wherein the α3β4/α4β2 nAChR subtype binding affinity ratio for said compound of formula I is greater than about 3000:1 in a nAChR binding assay.

In certain embodiments, the present invention relates to the aforementioned compound, wherein said compound of formula I has a Kᵢ of less than about 500 nM in an assay based on an α4β2 nAChR receptor.

In certain embodiments, the present invention relates to the aforementioned compound, wherein said compound of formula I has a Kᵢ of less than about 100 nM in an assay based on an α4β2 nAChR receptor.

In certain embodiments, the present invention relates to the aforementioned compound, wherein said compound of formula I has a Kᵢ of less than about 50 nM in an assay based on an α4β2 nAChR receptor.

In certain embodiments, the present invention relates to the aforementioned compound, wherein said compound of formula I has a Kᵢ of less than about 25 nM in an assay based on an α4β2 nAChR receptor.

In certain embodiments, the present invention relates to the aforementioned compound, wherein said compound of formula I has a Kᵢ of less than about 10 nM in an assay based on an α4β2 nAChR receptor.

Another aspect of the invention relates to a compound represented by formula II:

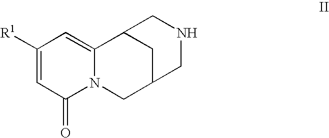

or a pharmaceutically acceptable salt thereof;

wherein

the stereochemical configuration at any stereocenter of said compound is R, S, or a mixture thereof;

R₁ is —OH, —SH, halogen, —CF₃, —CN, —NO₂, optionally substituted C₁-C₆ alkyl chain, optionally substituted benzyl, optionally substituted heteroaryl, optionally substituted cycloalkyl, —NH₂, di-[(C₁-C₆)alkylamino, (C₁-C₆) monoalkylamino, (C₆-C₁₀) arylamino, (C₃-C₈)cycloalkylamino, heteroarylamino, cycloheteroalkylamino; —C(O)R wherein R is H, optionally substituted (C₁-C₆)alkyl, optionally substituted aryl, or optionally substituted benzyl; —CO₂R wherein R is H, (C₁-C₆) alkyl, phenyl, or benzyl; —CON(R)₂ wherein each R is hydrogen, (C₁-C₆)alkyl or (C₆-C₁₀)aryl; —NHC(O)R, wherein R is optionally substituted alkyl (C₁-C₆ chain), optionally substituted aryl, or optionally substituted benzyl; —XR wherein X is O, S or N, and R is hydrogen, alkyl, or aryl bearing 0, 1 or 2 substituents; optionally benzene-fused (C₆-C₁₀) aryl; optionally benzene-fused (C₃-C₈)cycloalkyl; optionally benzene-fused heteroaryl wherein said heteroaryl group contains 5 to 10 atoms comprising one to four heteroatoms; optionally benzene-fused cycloheteroalkyl wherein said cycloheteroalkyl contains 4 to 8 atoms comprising one or two heteroatoms selected from group consisting of N, S and O; —CH₂XR, wherein X is O, S or N, and when X=O, R is selected from the group consisting of hydrogen, allyl, optionally substituted alkenyl, alkoxy methyl, cycloalkyloxy methyl, —C(O)R″ and aryl bearing 0, 1 or 2 substituents, wherein R″ is optionally substituted alkyl, optionally substituted aryl, or optionally substituted benzyl, when X=S or N, R is hydrogen, optionally substituted alkyl, optionally substituted aryl, —NH₂, di-[(C₁-C₆)alkylamino, (C₁-C₆)monoalkylamino, (C₆-C₁₀) arylamino, (C₃-C₈)cycloalkylamino, heteroarylamino, cycloheteroalkylamino or —NHC(O)R′″, wherein R′″ is optionally substituted (C₁-C₆)alkyl chain, optionally substituted aryl, optionally substituted benzyl; —(CH₂)ₙ—OCH₂-(10-Cytisine); —(CH₂)ₙ(10-Cytisine); alkenyl; alkynyl; wherein said alkenyl, alkynyl, and aryl are optionally substituted with halogen, CN, OH, hydroxymethyl, alkoxy, NO₂, amine, alkyl amine, or —NHC(O)R, wherein R is alkyl (C₁-C₆ chain), aryl, or benzyl; and n is 1, 2, 3, 4, 5, or 6.

In certain embodiments, the present invention relates to the aforementioned compound, wherein said compound of formula II is a single enantiomer.

In certain embodiments, the present invention relates to the aforementioned compound, wherein said compound of formula II is a single diastereomer.

Another aspect of the invention relates to a compound represented by formula III:

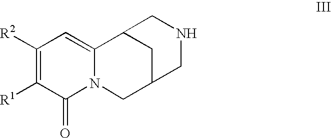

or a pharmaceutically acceptable salt thereof;

wherein

the stereochemical configuration at any stereocenter of said compound is R, S, or a mixture thereof;

R¹ and R² taken together form a 5-8 member ring containing 0, 1, 2, or 3 heteroatoms selected from the group consisting of N, O, and S; and said 5-8 member ring is optionally fused with an aryl or heteroaryl ring.

In certain embodiments, the present invention relates to 9-bromo-1,2,3,4,5,6-hexahydro-10-methyl-1,5-methano-pyrido[1,2-a][1,5]diazocin-8-one; 11-bromo-1,2,3,4,5,6-hexahydro-10-methyl-1,5-methano-pyrido[1,2-a][1,5]diazocin-8-one; 9-chloro-1,2,3,4,5,6-hexahydro-10-methyl-1,5-methano-pyrido[1,2-a][1,5]diazocin-8-one; 11-chloro-1,2,3,4,5,6-hexahydro-10-methyl-1,5-methano-pyrido[1,2-a][1,5]diazocin-8-one; 9-fluoro-1,2,3,4,5,6-hexahydro-10-methyl-1,5-methano-pyrido[1,2-a][1,5]diazocin-8-one; 11-fluoro-1,2,3,4,5,6-hexahydro-10-methyl-1,5-methano-pyrido[1,2-a][1,5]diazocin-8-one; 9,11-difluoro-1,2,3,4,5,6-hexahydro-10-methyl-1,5-methano-pyrido[1,2-a][1,5]diazocin-8-one; 9-ethyl-1,2,3,4,5,6-hexahydro-10-methyl-1,5-methano-pyrido[1,2-a][1,5]diazocin-8-one; 11-ethyl-1,2,3,4,5,6-hexahydro-10-methyl-1,5-methano-pyrido[1,2-a][1,5]diazocin-8-one; 9,11-diethyl-1,2,3,4,5,6-hexahydro-10-methyl-1,5-methano-pyrido[1,2-a][1,5]diazocin-8-one; 9,10-dimethyl-1,2,3,4,5,6-hexahydro-1,5-methano-pyrido[1,2-a][1,5]diazocin-8-one; 10,11-dimethyl-1,2,3,4,5,6-hexahydro-1,5-methano-pyrido[1,2-a][1,5]diazocin-8-one; 9,10,11-trimethyl-1,2,3,4,5,6-hexahydro-1,5-methano-pyrido[1,2-a][1,5]diazocin-8-one; 9-phenyl-1,2,3,4,5,6-hexahydro-10-methyl-1,5-methano-pyrido[1,2-a][1,5]diazocin-8-one; 11-phenyl-1,2,3,4,5,6-hexahydro-10-methyl-1,5-methano-pyrido[1,2-a][1,5]diazocin-8-one; 9,11-diphenyl-1,2,3,4,5,6-hexahydro-10-methyl-1,5-methano-pyrido[1,2-a][1,5]diazocin-8-one; 9-vinyl-1,2,3,4,5,6-hexahydro-10-methyl-1,5-methano-pyrido[1,2-a][1,5]diazocin-8-one; 11-vinyl-1,2,3,4,5,6-hexahydro-10-methyl-1,5-methano-pyrido[1,2-a][1,5]diazocin-8-one; 9,11-divinyl-1,2,3,4,5,6-hexahydro-10-methyl-1,5-methano-pyrido[1,2-a][1,5]diazocin-8-one; 9-bromo-3,10-dimethyl-1,2,3,4,5,6-hexahydro-1,5-methano-pyrido[1,2-a][1,5]diazocin-8-one; 3-benzyl-9-bromo-1,2,3,4,5,6-hexahydro-10-methyl-1,5-methano-pyrido[1,2-a][1,5]diazocin-8-one; 3-benzyl-9-chloro-1,2,3,4,5,6-hexahydro-10-methyl-1,5-methano-pyrido[1,2-a][1,5]diazocin-8-one; 9-morpholino-1,2,3,4,5,6-hexahydro-10-methyl-1,5-methano-pyrido[1,2a][1,5]diazocin-8-one; 9-benzylamino-1,2,3,4,5,6-hexahydro-10-methyl-1,5-methano-pyrido[1,2a][1,5]diazocin-8-one; 9-pyrrolidino-1,2,3,4,5,6-hexahydro-10-methyl-1,5-methano-pyrido[1,2a][1,5]diazocin-8-one; 9-dimethylamino-1,2,3,4,5,6-hexahydro-10-methyl-1,5-methano-pyrido[1,2a][1,5]diazocin-8-one; 9-acetyl-1,2,3,4,5,6-hexahydro-10-methyl-1,5-methano-pyrido[1,2a][1,5]diazocin-8-one; 9-(2-tetrahydrofuranyl)-1,2,3,4,5,6-hexahydro-10-methyl-1,5-methano-pyrido[1,2a][1,5]diazocin-8-one; 9-iodo-1,2,3,4,5,6-hexahydro-10-methyl-1,5-methano-pyrido[1,2a][1,5]diazocin-8-one; 9-cyano-1,2,3,4,5,6-hexahydro-10-methyl-1,5-methano-pyrido[1,2a][1,5]diazocin-8-one; 9-ethynyl-1,2,3,4,5,6-hexahydro-10-methyl-1,5-methano-pyrido[1,2a][1,5]diazocin-8-one; 9-(2-propenyl)-1,2,3,4,5,6-hexahydro-10-methyl-1,5-methano-pyrido[1,2a][1,5]diazocin-8-one; 9-(2-propyl)-1,2,3,4,5,6-hexahydro-10-methyl-1,5-methano-pyrido[1,2a][1,5]diazocin-8-one; 11-phenyl-1,2,3,4,5,6-hexahydro-10-hydroxymethyl-1,5-methano-pyrido[1,2-a][1,5]diazocin-8-one; 9,11-diphenyl-1,2,3,4,5,6-hexahydro-10-hydroxymethyl-1,5-methano-pyrido[1,2-a][1,5]diazocin-8-one; 9-vinyl-1,2,3,4,5,6-hexahydro-10-hydroxymethyl-1,5-methano-pyrido[1,2-a][1,5]diazocin-8-one; 11-vinyl-1,2,3,4,5,6-hexahydro-10-hydroxymethyl-1,5-methano-pyrido[1,2-a][1,5]diazocin-8-one; 9,11-divinyl-1,2,3,4,5,6-hexahydro-10-hydroxymethyl-1,5-methano-pyrido[1,2-a][1,5]diazocin-8-one; 3-benzyl-9-bromo-1,2,3,4,5,6-hexahydro-10-hydroxymethyl-1,5-methano-pyrido[1,2-a][1,5]diazocin-8-one; 3-benzyl-9-chloro-1,2,3,4,5,6-hexahydro-10-hydroxymethyl-1,5-methano-pyrido[1,2-a][1,5]diazocin-8-one; 9-morpholino-1,2,3,4,5,6-hexahydro-10-hydroxymethyl-1,5-methano-pyrido[1,2a][1,5]diazocin-8-one; 9-benzylamino-1,2,3,4,5,6-hexahydro-10-hydroxymethyl-1,5-methano-pyrido[1,2a][1,5]diazocin8-one; 9-pyrrolidino-1,2,3,4,5,6-hexahydro-10-hydroxymethyl-1,5-methano-pyrido[1,2a][1,5]diazocin-8-one; 9-dimethylamino-1,2,3,4,5,6-hexahydro-10-hydroxymethyl-1,5-methano-pyrido[1,2a][1,5]diazocin-8-one; 9-acetyl-1,2,3,4,5,6-hexahydro-10-hydroxymethyl-1,5-methano-pyrido[1,2a][1,5]diazocin-8-one; 9-(2-tetrahydrofuranyl)-1,2,3,4,5,6-hexahydro-10-hydroxymethyl-1,5-methano-pyrido[1,2a][1,5]diazocin-8-one; 9-iodo-1,2,3,4,5,6-hexahydro-10-hydroxymethyl-1,5-methano-pyrido[1,2a][1,5]diazocin-8-one; 9-cyano-1,2,3,4,5,6-hexahydro-10-hydroxymethyl-1,5-methano-pyrido[1,2a][1,5]diazocin-8-one; 9-ethynyl-1,2,3,4,5,6-hexahydro-10-hydroxymethyl-1,5-methano-pyrido[1,2a][1,5]diazocin-8-one; 9-(2-propenyl)-1,2,3,4,5,6-hexahydro-10-hydroxymethyl-1,5-methano-pyrido[1,2a][1,5]diazocin-8-one; 9-(2-propyl)-1,2,3,4,5,6-hexahydro-10-hydroxymethyl-1,5-methano-pyrido[1,2a][1,5]diazocin-8-one; 9-carbomethoxy-1,2,3,4,5,6-hexahydro-10-hydroxymethyl-1,5-methano-pyrido[1,2a][1,5]diazocin-8-one; 9-carboxyaldehyde-1,2,3,4,5,6-hexahydro-10-hydroxymethyl-1,5-methano-pyrido[1,2a][1,5]diazocin-8-one; 9-(2-methoxyphenyl)-1,2,3,4,5,6-hexahydro-10-hydroxymethyl-1,5-methano-pyrido[1,2a][1,5]diazocin-8-one; 9-(2,6-difluorophenyl)-1,2,3,4,5,6-hexahydro-10-hydroxymethyl-1,5-methano-pyrido[1,2a][1,5]diazocin-8-one; 9-[2-(1,1,1-trifluoromethylphenyl)-1,2,3,4,5,6-hexahydro-10-hydroxymethyl-1,5-methano-pyrido[1,2a][1,5]diazocin-8-one; 9-(4-methoxyphenyl)-1,2,3,4,5,6-hexahydro-10-hydroxymethyl-1,5-methano-pyrido[1,2a][1,5]diazocin-8-one; 9-(2-ethoxy-5-methylphenyl)-1,2,3,4,5,6-hexahydro-10-hydroxymethyl-1,5-methano-pyrido[1,2a][1,5]diazocin-8-one; 9-(2-benzofuranyl)-1,2,3,4,5,6-hexahydro-10-hydroxymethyl-1,5-methano-pyrido[1,2a][1,5]diazocin-8-one; 9-(2-thienyl)-1,2,3,4,5,6-hexahydro-10-hydroxymethyl-1,5-methano-pyrido[1,2a][1,5]diazocin-8-one; 9-(3-thienyl)-1,2,3,4,5,6-hexahydro-10-hydroxymethyl-1,5-methano-pyrido[1,2a][1,5]diazocin-8-one; 9-[3-(4-methylthienyl)]-1,2,3,4,5,6-hexahydro-10-hydroxymethyl-1,5-methano-pyrido[1,2a][1,5]diazocin-8-one; 9-[2-(3-methylthienyl)]-1,2,3,4,5,6-hexahydro-10-hydroxymethyl-1,5-methano-pyrido[1,2a][diazocin-8-one; 9-[3-(2-fluoropyridyl)]-1,2,3,4,5,6-hexahydro-10-hydroxymethyl-1,5-methano-pyrido[1,2a][1,5]diazocin-8-one; 9-(2-pyridyl)-1,2,3,4,5,6-hexahydro-10-hydroxymethyl-1,5-methano-pyrido[1,2a][1,5]diazocin-8-one; 9-(2-furanyl)-1,2,3,4,5,6-hexahydro-10-hydroxymethyl-1,5-methano-pyrido[1,2a][1,5]diazocin-8-one; 9-(3-furanyl)-1,2,3,4,5,6-hexahydro-10-hydroxymethyl-1,5-methano-pyrido[1,2a][1,5]diazocin-8-one; 9-(2-trifluoromethylphenyl)-1,2,3,4,5,6-hexahydro-10-hydroxymethyl-1,5-methano-pyrido[1,2a][1,5]diazocin-8-one; 9-(4-trifluoromethylphenyl)-1,2,3,4,5,6-hexahydro-10-hydroxymethyl-1,5-methano-pyrido[1,2a][1,5]diazocin-8-one; 9-phenyl-1,2,3,4,5,6-hexahydro-10-hydroxymethyl-1,5-methano-pyrido[1,2a][1,5]diazocin-8-one; 11-phenyl-1,2,3,4,5,6-hexahydro-10-hydroxymethyl-1,5-methano-pyrido[1,2a][1,5]diazocin-8-one; 9-(2-methylphenyl)-1,2,3,4,5,6-hexahydro-10-hydroxymethyl-1,5-methano-pyrido[1,2a][1,5]diazocin-8-one; 9-(3-acetylphenyl)-1,2,3,4,5,6-hexahydro-10-hydroxymethyl-1,5-methano-pyrido[1,2a][1,5]diazocin-8-one; 9-(2-chlorophenyl)-1,2,3,4,5,6-hexahydro-10-hydroxymethyl-1,5-methano-pyrido[1,2a][1,5]diazocin-8-one; 9-(3,4-dichlorophenyl)-1,2,3,4,5,6-hexahydro-10-hydroxymethyl-1,5-methano-pyrido[1,2a][1,5]diazocin-8-one; 9-(2-fluorophenyl)-1,2,3,4,5,6-hexahydro-10-hydroxymethyl-1,5-methano-pyrido[1,2a][1,5]diazocin-8-one; 9-(4-fluorophenyl)-1,2,3,4,5,6-hexahydro-10-hydroxymethyl-1,5-methano-pyrido[1,2a][1,5]diazocin-8-one; 9-(3-fluorophenyl)-1,2,3,4,5,6-hexahydro-10-hydroxymethyl-1,5-methano-pyrido[1,2a][1,5]diazocin-8-one; 9-(3,5-difluorophenyl)-1,2,3,4,5,6-hexahydro-10-hydroxymethyl-1,5-methano-pyrido[1,2a][1,5]diazocin-8-one; 9-(2,4-difluorophenyl)-1,2,3,4,5,6-hexahydro-10-hydroxymethyl-1,5-methano-pyrido[1,2a][1,5]diazocin-8-one; 9-(2-fluoro4-chlorophenyl)-1,2,3,4,5,6-hexahydro-10-hydroxymethyl-1,5-methano-pyrido[1,2a][1,5]diazocin-8-one; 9-(2-fluoro4-methoxyphenyl)-1,2,3,4,5,6-hexahydro-1,5-methano-pyrido[1,2a][1,5]diazocin-8-one; 9-(2,5-difluorophenyl)-1,2,3,4,5,6-hexahydro-10-hydroxymethyl-1,5-methano-pyrido[1,2a][1,5]diazocin-8-one; N-t-BOC-9-iodo-1,2,3,4,5,6-hexahydro-10-hydroxymethyl-1,5-methano-pyrido[1,2a][1,5]diazocin-8-one; N-cBz-9-iodo-1,2,3,4,5,6-hexahydro-10-hydroxymethyl-1,5-methano-pyrido[1,2a][1,5]diazocin-8-one; N-trifluoroacety-9-iodo-1,2,3,4,5,6-hexahydro-10-hydroxymethyl-1,5-methano-pyrido[1,2a][1,5]diazocin-8-one; N-trifluoroacety-9-bromo-1,2,3,4,5,6-hexahydro-10-hydroxymethyl-1,5-methano-pyrido[1,2a][1,5]diazocin-8-one; N-acetyl-9-iodo-1,2,3,4,5,6-hexahydro-10-hydroxymethyl-1,5-methano-pyrido[1,2a][1,5]diazocin-8-one; N-tBOC-9-boronic acid-1,2,3,4,5,6-hexahydro-10-hydroxymethyl-1,5-methano-pyrido[1,2a][1,5]diazocin-8-one; N-acetyl-9-bromo-1,2,3,4,5,6-hexahydro-10-hydroxymethyl-1,5-methano-pyrido[1,2a][1,5]diazocin-8-one; N-t-BOC-9-acetyl-1,2,3,4,5,6-hexahydro-10-hydroxymethyl-1,5-methano-pyrido[1,2a][1,5]diazocin-8-one; N-trifluoroacety-9-acetyl-1,2,3,4,5,6-hexahydro-10-hydroxymethyl-1,5-methano-pyrido[1,2a][1,5]diazocin-8-one; N-cBz-9-acetyl-1,2,3,4,5,6-hexahydro-10-hydroxymethyl-1,5-methano-pyrido[1,2a][1,5]diazocin-8-one; N-acetyl-9-acetyl-1,2,3,4,5,6-hexahydro-10-hydroxymethyl-1,5-methano-pyrido[1,2a][1,5]diazocin-8-one; N-t-BOC-9-cyano-1,2,3,4,5,6-hexahydro-10-hydroxymethyl-1,5-methano-pyrido[1,2a][1,5]diazocin-8-one; N-t-BOC-9-ethyny-1,2,3,4,5,6-hexahydro-10-hydroxymethyl-1,5-methano-pyrido[1,2a][1,5]diazocin-8-one; N-t-BOC-9-dimethylamino-1,2,3,4,5,6-hexahydro-10-hydroxymethyl-1,5-methano-pyrido[1,2a][1,5]diazocin-8-one; N-t-BOC-9-(2-propenyl)-1,2,3,4,5,6-hexahydro-10-hydroxymethyl-1,5-methano-pyrido[1,2a][1,5]diazocin-8-one; N-t-BOC-9-(2-propyl)-1,2,3,4,5,6-hexahydro-10-hydroxymethyl-1,5-methano-pyrido[1,2a][1,5]diazocin-8-one; N-t-BOC-9-(2-(1,2-propanediol)-1,2,3,4,5,6-hexahydro-10-hydroxymethyl-1,5-methano-pyrido[1,2a][1,5]diazocin-8-one; N-t-BOC-9-carbomethoxy-1,2,3,4,5,6-hexahydro-10-hydroxymethyl-1,5-methano-pyrido[1,2a][1,5]diazocin-8-one; N-t-BOC-9-carboxyaldehyde-1,2,3,4,5,6-hexahydro-10-hydroxymethyl-1,5-methano-pyrido[1,2a][1,5diazocin-8-one; N-t-BOC-9-bromo-1,2,3,4,5,6-hexahydro-10-hydroxymethyl-1,5-methano-pyrido[1,2a][1,5]diazocin-8-one; N-t-BOC-11-bromo-1,2,3,4,5,6-hexahydro-10-hydroxymethyl-1,5-methano-pyrido[1,2-a][1,5]diazocin-8-one; N-t-BOC-9,11-dibromo-1,2,3,4,5,6-hexahydro-10-hydroxymethyl-1,5-methano-pyrido[1,2-a][1,5]diazocin-8-one; N-t-BOC-9-chloro-1,2,3,4,5,6-hexahydro-10-hydroxymethyl-1,5-methano-pyrido[1,2-a][1,5]diazocin-8-one; N-t-BOC-11-chloro-1,2,3,4,5,6-hexahydro-10-hydroxymethyl-1,5-methano-pyrido[1,2-a][1,5]diazocin-8-one; N-t-BOC-9,11-dichloro-1,2,3,4,5,6-hexahydro-10-hydroxymethyl-1,5-methano-pyrido[1,2-a][1,5]diazocin-8-one; N-t-BOC-9-fluoro-1,2,3,4,5,6-hexahydro-10-hydroxymethyl-1,5-methano-pyrido[1,2-a][1,5]diazocin-8-one; N-t-BOC-11-fluoro-1,2,3,4,5,6-hexahydro-10-hydroxymethyl-1,5-methano-pyrido[1,2-a][1,5]diazocin-8-one; N-t-BOC-9,11-difluoro-1,2,3,4,5,6-hexahydro-10-hydroxymethyl-1,5-methano-pyrido[1,2-a][1,5]diazocin-8-one, or a pharmaceutically acceptable salt thereof.

Another aspect of the invention relates to a pharmaceutical composition comprising any one of the aforementioned compounds and a pharmaceutically acceptable excipient.

### Methods of the Invention

One aspect of the invention relates to a method of modulating a nicotinic ACh receptor in a mammal, comprising the step of administering to a mammal an effective amount of a compound of formula I:

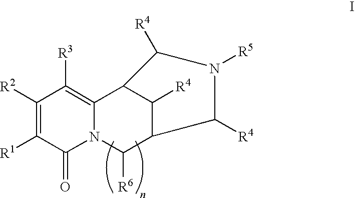

or a pharmaceutically acceptable salt thereof;

wherein

the stereochemical configuration at any stereocenter of said compound is R, S, or a mixture thereof;

R¹, R³, and R⁶ represent independently for each occurrence H, alkyl, cycloalkyl, alkenyl, cycloalkenyl, alkynyl, aryl, aralkyl, halogen, cyano, nitro, —OR⁷, —N(R⁷)₂, —SR⁷, —OC(O)R⁷, —N(R⁷)C(O)R⁷, —SC(O)R⁷, or —(C(R⁸)₂)ₚCR⁸═C(R⁸)₂; wherein said alkyl, cycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷;

R² is alkyl, cycloalkyl, heterocycloalkyl, alkenyl, cycloalkenyl, alkynyl, aryl, aralkyl, halogen, cyano, nitro, —OR⁷, —N(R⁷)₂, —S, —OC(O)R⁷, —N(R⁷)C(O)R⁷, —SC(O)R⁷, —C(O)R⁷, —CO₂R⁷, —C(O)N(R⁷)₂, —OC(O)R⁷, —N(R⁷)C(O)R⁷, or —(C(R⁸)₂)ₚCR⁸═C(R⁸)₂; wherein said alkyl, cycloalkyl, heterocycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷; or R² and R³ taken together form a 5-7 member ring containing 0, 1, or 2 heteroatoms selected from the group consisting of O and N; or R¹ and R² taken together form a 5-7 member ring containing 0, 1, or 2 heteroatoms selected from the group consisting of O and N; or R² is

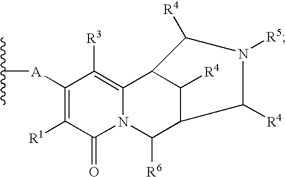

R⁴ represents independently for each occurrence H, alkyl, alkenyl, halogen, —OR⁷, —N(R⁷)₂, or —(C(R⁸)₂)ₚCR⁸═C(R⁸)₂;

R⁵ is H, alkyl, cycloalkyl, alkenyl, cycloalkenyl, alkynyl, aryl, aralkyl, —C(O)R⁷, —CO₂R⁷, —C(O)N(R⁷)₂, or —(C(R⁸)₂)ₚCR⁸═C(R⁸)₂; wherein said alkyl, cycloalkyl, alkenyl, cycloalkenyl, alkynyl, aryl, and aralkyl are optionally substituted with one or more of halogen, —OR⁷, —N(R⁷)₂, —SR⁷, —C(O)R⁷, —CO₂R⁷, —C(O)N(R⁷)₂, —OC(O)R⁷, or —N(R⁷)C(O)R⁷;

R⁷ represents independently for each occurrence H, alkyl, cycloalkyl, heterocycloalkyl, alkenyl, cycloalkenyl, allyl, alkynyl, aryl, aralkyl, alkoxyalkyl, aryloxyalkyl, or cycloalkyloxyalkyl;

R⁸ represents independently for each occurrence H or (C₁-C₆)alkyl;

A is an alkyl diradical, alkenyl diradical, aryl diradical, aralkyl diradical, or —(C(R⁸)₂)ₘ—X—(C(R⁸)₂)ₘ—;

X is O, —N(R⁷)—, or S;

m and p represent independently for each occurrence 1, 2, 3, 4, 5, or 6; and

n is 1 or 2.

In certain embodiments, the present invention relates to the aforementioned method, wherein R¹ represents independently for each occurrence H, alkyl, alkenyl, aryl, aralkyl, halogen, —C(O)R⁷, —CO₂R⁷, —C(O)N(R⁷)₂, or —(C(R⁸)₂)ₚCR⁸═C(R⁸)₂.

In certain embodiments, the present invention relates to the aforementioned method, wherein R¹ represents independently for each occurrence H or (C₁-C₆)alkyl.

In certain embodiments, the present invention relates to the aforementioned method, wherein R¹ represents independently for each occurrence H.

In certain embodiments, the present invention relates to the aforementioned method, wherein R² is alkyl, cycloalkyl, heterocycloalkyl, alkenyl, cycloalkenyl, alkynyl, aryl, aralkyl, halogen, cyano, nitro, —OR⁷, —N(R⁷)₂, —SR⁷, —C(O)R⁷, —CO₂R⁷, —C(O)N(R⁷)₂, —OC(O)R⁷, —N(R⁷)C(O)R⁷, or —(C(R⁸)₂)ₚCR⁸═C(R⁸)₂; wherein said alkyl, cycloalkyl, heterocycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷.

In certain embodiments, the present invention relates to the aforementioned method, wherein R² is alkyl, cycloalkyl, heterocycloalkyl, alkenyl, aryl, aralkyl, halogen, cyano, —C(O)R⁷, —CO₂R⁷, —C(O)N(R⁷)₂, or —(C(R⁸)₂)ₚCR⁸═C(R⁸)₂; wherein said alkyl, cycloalkyl, heterocycloalkyl, alkenyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷.

In certain embodiments, the present invention relates to the aforementioned method, wherein R² is alkyl, cycloalkyl, alkenyl, aryl, or —(C(R⁸)₂)ₚCR⁸═C(R⁸)₂; wherein said alkyl, cycloalkyl, alkenyl, and aryl are optionally substituted with one or more of halogen, —OR⁷, —N(R⁷)₂, or —SR⁷; wherein R⁷ is H or (C₁-C₆)alkyl.

In certain embodiments, the present invention relates to the aforementioned method, wherein R² is alkyl or cycloalkyl; wherein said alkyl and cycloalkyl are optionally substituted with one or more of halogen, —OR⁷, —N(R⁷)₂, or —SR⁷; wherein R⁷ is H or (C₁-C₆)alkyl.

In certain embodiments, the present invention relates to the aforementioned method, wherein R² is (C₁-C₆)alkyl optionally substituted with —OR⁷, —N(R⁷)₂, or —SR⁷; wherein R⁷ is H or (C₁-C₆)alkyl.

In certain embodiments, the present invention relates to the aforementioned method, wherein R² is (C₁-C₆)alkyl optionally substituted with —OR⁷; wherein R⁷ is H or (C₁-C₆)alkyl.

In certain embodiments, the present invention relates to the aforementioned method, wherein R² is (C₁-C₆)alkyl.

In certain embodiments, the present invention relates to the aforementioned method, wherein R² is methyl, ethyl, propyl, isopropyl, butyl, isobutyl, sec-butyl, or pentyl optionally substituted with —OR⁷; wherein R⁷ is H or (C₁-C₆)alkyl.

In certain embodiments, the present invention relates to the aforementioned method, wherein R² is methyl, ethyl, propyl, isopropyl, butyl, isobutyl, sec-butyl, or pentyl.

In certain embodiments, the present invention relates to the aforementioned method, wherein R² is methyl.

In certain embodiments, the present invention relates to the aforementioned method, wherein R² is —CH₂OH.

In certain embodiments, the present invention relates to the aforementioned method, wherein R³ represents independently for each occurrence H, alkyl, alkenyl, aryl, aralkyl, halogen, —C(O)R⁷, —CO₂R⁷, —C(O)N(R⁷)₂, or —(C(R⁸)₂)ₚCR⁸═C(R⁸)₂.

In certain embodiments, the present invention relates to the aforementioned method, wherein R³ represents independently for each occurrence H or (C₁-C₆)alkyl.

In certain embodiments, the present invention relates to the aforementioned method, wherein R³ represents independently for each occurrence H.

In certain embodiments, the present invention relates to the aforementioned method, wherein R⁴ represents independently for each occurrence H or alkyl.

In certain embodiments, the present invention relates to the aforementioned method, wherein R⁴ is H.

In certain embodiments, the present invention relates to the aforementioned method, wherein R⁵ is H, alkyl, cycloalkyl, aryl, aralkyl, —C(O)R⁷, —CO₂R⁷, —C(O)N(R⁷)₂, or —(C(R⁸)₂)ₚCR⁸═C(R⁸)₂.

In certain embodiments, the present invention relates to the aforementioned method, wherein R⁵ is H, alkyl, or benzyl.

In certain embodiments, the present invention relates to the aforementioned method, wherein R⁵ is H.

In certain embodiments, the present invention relates to the aforementioned method, wherein R⁶ represents independently for each occurrence H, alkyl, alkenyl, aryl, aralkyl, halogen, —C(O)R⁷, —CO₂R⁷, —C(O)N(R⁷)₂, or —(C(R⁸)₂)ₚCR⁸═C(R⁸)₂.

In certain embodiments, the present invention relates to the aforementioned method, wherein R⁶ represents independently for each occurrence H or (C₁-C₆)alkyl.

In certain embodiments, the present invention relates to the aforementioned method, wherein R⁶ represents independently for each occurrence H.

In certain embodiments, the present invention relates to the aforementioned method, wherein R⁷ represents independently for each occurrence H, alkyl, cycloalkyl, heterocycloalkyl, alkenyl, cycloalkenyl, allyl, alkynyl, aryl, or aralkyl.

In certain embodiments, the present invention relates to the aforementioned method, wherein R⁷ represents independently for each occurrence H, alkyl, cycloalkyl, aryl, or aralkyl.

In certain embodiments, the present invention relates to the aforementioned method, wherein R⁷ represents independently for each occurrence H, alkoxymethyl, aryloxymethyl, or cycloalkyloxymethyl

In certain embodiments, the present invention relates to the aforementioned method, wherein R⁷ represents independently for each occurrence H or alkyl.

In certain embodiments, the present invention relates to the aforementioned method, wherein R⁷ is H.

In certain embodiments, the present invention relates to the aforementioned method, wherein n is 1.

In certain embodiments, the present invention relates to the aforementioned method, wherein R¹, R³, R⁴, R⁵, and R⁶ represent independently for each occurrence H or alkyl.

In certain embodiments, the present invention relates to the aforementioned method, wherein R¹, R³, R⁴, R⁵, and R⁶ represent independently for each occurrence H or alkyl; and n is 1.

In certain embodiments, the present invention relates to the aforementioned method, wherein R¹, R³, R⁴, R⁵, and R⁶ represent independently for each occurrence H or alkyl; n is 1; and R² is alkyl, cycloalkyl, heterocycloalkyl, alkenyl, aryl, aralkyl, halogen, cyano, —C(O)R⁷, —CO₂R⁷, —C(O)N(R⁷)₂, or —(C(R⁸)₂)ₚCR⁸═C(R⁸)₂; wherein said alkyl, cycloalkyl, heterocycloalkyl, alkenyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷.

In certain embodiments, the present invention relates to the aforementioned method, wherein R¹, R³, R⁴, R⁵, and R⁶ represent independently for each occurrence H or alkyl; n is 1; R² is alkyl, cycloalkyl, alkenyl, aryl, or —(C(R⁸)₂)ₚCR⁸═C(R⁸)₂; wherein said alkyl, cycloalkyl, alkenyl, and aryl are optionally substituted with one or more of halogen, —OR⁷, —N(R⁷)₂, or —SR⁷; and R⁷ is H or (C₁-C₆)alkyl.

In certain embodiments, the present invention relates to the aforementioned method, wherein R¹, R³, R⁴, R⁵, and R⁶ represent independently for each occurrence H or alkyl; n is 1; R² represents independently for each occurrence (C₁-C₆)alkyl optionally substituted with —OR⁷, —N(R⁷)₂, or —SR⁷; and R⁷ is H or (C₁-C₆)alkyl.

In certain embodiments, the present invention relates to the aforementioned method, wherein R¹, R³, R⁴, R⁵, and R⁶ are H; n is 1; R² represents independently for each occurrence methyl, ethyl, propyl, isopropyl, butyl, isobutyl, sec-butyl, or pentyl optionally substituted with —OR⁷; and R⁷ is H or (C₁-C₆)alkyl.

In certain embodiments, the present invention relates to the aforementioned method, wherein R¹, R³, R⁴, R⁵, and R⁶ are H; n is 1; and R² is methyl.

In certain embodiments, the present invention relates to the aforementioned method, wherein R¹, R³, R⁴, R⁵, and R⁶ are H; n is 1; and R² is —CH₂OH.

In certain embodiments, the present invention relates to the aforementioned method, wherein the α3β4/α4β2 nAChR subtype binding affinity ratio for said compound of formula I is greater than about 500:1 in a nAChR binding assay.

In certain embodiments, the present invention relates to the aforementioned method, wherein the α3β4/α4β2 nAChR subtype binding affinity ratio for said compound of formula I is greater than about 1000:1 in a nAChR binding assay.

In certain embodiments, the present invention relates to the aforementioned method, wherein the α3β4/α4β2 nAChR subtype binding affinity ratio for said compound of formula I is greater than about 2000:1 in a nAChR binding assay.

In certain embodiments, the present invention relates to the aforementioned method, wherein the α3β4/α4β2 nAChR subtype binding affinity ratio for said compound of formula I is greater than about 3000:1 in a nAChR binding assay.

In certain embodiments, the present invention relates to the aforementioned method, wherein said compound of formula I has a Kᵢ of less than about 500 nM in an assay based on an α4β2 nAChR receptor.

In certain embodiments, the present invention relates to the aforementioned method, wherein said compound of formula I has a Kᵢ of less than about 100 nM in an assay based on an α4β2 nAChR receptor.

In certain embodiments, the present invention relates to the aforementioned method, wherein said compound of formula I has a Kᵢ of less than about 50 nM in an assay based on an α4β2 nAChR receptor.

In certain embodiments, the present invention relates to the aforementioned method, wherein said compound of formula I has a Kᵢ of less than about 25 nM in an assay based on an α4β2 nAChR receptor.

In certain embodiments, the present invention relates to the aforementioned method, wherein said compound of formula I has a Kᵢ of less than about 10 nM in an assay based on an α4β2 nAChR receptor.

In certain embodiments, the present invention relates to the aforementioned method, wherein said nicotinic ACh receptor is a neuronal nicotinic ACh receptor.

In certain embodiments, the present invention relates to the aforementioned method, wherein said receptor is an α4β2 nAChR receptor.

In certain embodiments, the present invention relates to the aforementioned method, wherein said receptor is an α2β2 nAChR receptor.

In certain embodiments, the present invention relates to the aforementioned method, wherein said receptor is an α2β4 nAChR receptor.

In certain embodiments, the present invention relates to the aforementioned method, wherein said receptor is an α3β2 nAChR receptor.

In certain embodiments, the present invention relates to the aforementioned method, wherein said receptor is an α3β4 nAChR receptor.

In certain embodiments, the present invention relates to the aforementioned method, wherein said receptor is an α4β4 nAChR receptor.

In certain embodiments, the present invention relates to the aforementioned method, wherein the mammal is a primate, equine, canine, or feline.

In certain embodiments, the present invention relates to the aforementioned method, wherein the mammal is a human.

In certain embodiments, the present invention relates to the aforementioned method, wherein the compound is administered orally.

In certain embodiments, the present invention relates to the aforementioned method, wherein the compound is administered intravenously.

In certain embodiments, the present invention relates to the aforementioned method, wherein the compound is administered sublingually.

In certain embodiments, the present invention relates to the aforementioned method, wherein the compound is administered ocularly.

In certain embodiments, the present invention relates to the aforementioned method, wherein the compound is administered transdermally.

In certain embodiments, the present invention relates to the aforementioned method, wherein the compound is administered rectally.

In certain embodiments, the present invention relates to the aforementioned method, wherein the compound is administered vaginally.

In certain embodiments, the present invention relates to the aforementioned method, wherein the compound is administered topically.

In certain embodiments, the present invention relates to the aforementioned method, wherein the compound is administered intramuscularly.

In certain embodiments, the present invention relates to the aforementioned method, wherein the compound is administered subcutaneously.

In certain embodiments, the present invention relates to the aforementioned method, wherein the compound is administered buccally.

In certain embodiments, the present invention relates to the aforementioned method, wherein the compound is administered nasally.

Another aspect of the invention relates to a method of modulating a nicotinic ACh receptor in a mammal, comprising the step of administering to a mammal an effective amount of a compound of formula II:

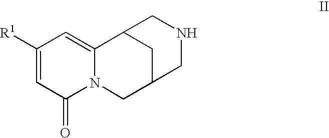

or a pharmaceutically acceptable salt thereof;

wherein

the stereochemical configuration at any stereocenter of said compound is R, S, or a mixture thereof;

R₁ is —OH, —SH, halogen, —CF₃, —CN, —NO₂, optionally substituted C₁-C₆ alkyl chain, optionally substituted benzyl, optionally substituted heteroaryl, optionally substituted cycloalkyl, —NH₂, di-[(C₁-C₆)alkylamino, (C₁-C₆) monoalkylamino, (C₆-C₁₀) arylamino, (C₃-C₈)cycloalkylamino, heteroarylamino, cycloheteroalkylamino; —C(O)R wherein R is H, optionally substituted (C₁-C₆)alkyl, optionally substituted aryl, or optionally substituted benzyl; —CO₂R wherein R is H, (C₁-C₆) alkyl, phenyl, or benzyl; —CON(R)₂ wherein each R is hydrogen, (C₁-C₆)alkyl or (C₆-C₁₀)aryl; —NHC(O)R, wherein R is optionally substituted alkyl (C₁-C₆ chain), optionally substituted aryl, or optionally substituted benzyl; —XR wherein X is O, S or N, and R is hydrogen, alkyl, or aryl bearing 0, 1 or 2 substituents; optionally benzene-fused (C₆-C₁₀) aryl; optionally benzene-fused (C₃-C₈)cycloalkyl; optionally benzene-fused heteroaryl wherein said heteroaryl group contains 5 to 10 atoms comprising one to four heteroatoms; optionally benzene-fused cycloheteroalkyl wherein said cycloheteroalkyl contains 4 to 8 atoms comprising one or two heteroatoms selected from group consisting of N, S and O; —CH₂XR, wherein X is O, S or N, and when X=O, R is selected from the group consisting of hydrogen, allyl, optionally substituted alkenyl, alkoxy methyl, cycloalkyloxy methyl, —C(O)R″ and aryl bearing 0, 1 or 2 substituents, wherein R″ is optionally substituted alkyl, optionally substituted aryl, or optionally substituted benzyl, when X=S or N, R is hydrogen, optionally substituted alkyl, optionally substituted aryl, —NH₂, di-[(C₁-C₆)alkylamino, (C₁-C₆)monoalkylamino, (C₆-C₁₀) arylamino, (C₃-C₈)cycloalkylamino, heteroarylamino, cycloheteroalkylamino or —NHC(O)R′″, wherein R′″ is optionally substituted (C₁-C₆)alkyl chain, optionally substituted aryl, optionally substituted benzyl; —(CH₂)ₙ—OCH₂-(10-Cytisine); —(CH₂)ₙ(10-Cytisine); alkenyl; alkynyl; wherein said alkenyl, alkynyl, and aryl are optionally substituted with halogen, CN, OH, hydroxymethyl, alkoxy, NO₂, amine, alkyl amine, or —NHC(O)R, wherein R is alkyl (C₁-C₆ chain), aryl, or benzyl; and n is 1, 2, 3, 4, 5, or 6.

In certain embodiments, the present invention relates to the aforementioned method, wherein said compound of formula II is a single enantiomer.

In certain embodiments, the present invention relates to the aforementioned method, wherein said compound of formula II is a single diastereomer.

Another aspect of the invention relates to a method of modulating a nicotinic ACh receptor in a mammal, comprising the step of administering to a mammal an effective amount of a compound of formula III:

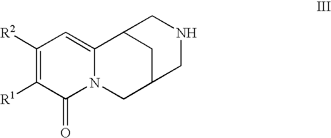

or a pharmaceutically acceptable salt thereof;

wherein

the stereochemical configuration at any stereocenter of said compound is R, S, or a mixture thereof;

R¹ and R² taken together form a 5-8 member ring containing 0, 1, 2, or 3 heteroatoms selected from the group consisting of N, O, and S; and said 5-8 member ring is optionally fused with an aryl or heteroaryl ring.

Another aspect of the invention relates to a method of treating a mammal suffering from Alzheimer's disease, Parkinson's disease, dyskinesias, Tourette's syndrome, schizophrenia, attention deficit disorder, anxiety, pain, depression, obsessive compulsive disorder, chemical substance abuse, alcoholism, memory deficit, pseudodementia, Ganser's syndrome, migraine pain, bulimia, obesity, premenstrual syndrome or late luteal phase syndrome, tobacco abuse, post-traumatic syndrome, social phobia, chronic fatigue syndrome, premature ejaculation, erectile difficulty, anorexia nervosa, disorders of sleep, autism, mutism, avoidance learning, or trichotillomania, comprising the step of administering to a mammal in need thereof a therapeutically effective amount of a compound of Formula I:

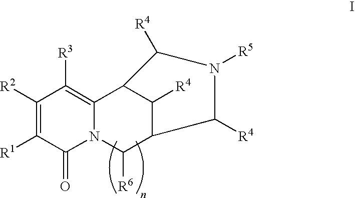

or a pharmaceutically acceptable salt thereof;

wherein

the stereochemical configuration at any stereocenter of said compound is R, S, or a mixture thereof;

R¹, R³, and R⁶ represent independently for each occurrence H, alkyl, cycloalkyl, alkenyl, cycloalkenyl, alkynyl, aryl, aralkyl, halogen, cyano, nitro, —OR⁷, —N(R⁷)₂, —SR⁷, —C(O)R⁷, —CO₂R⁷, —C(O)N(R⁷)₂, —OC(O)R⁷, —N(R⁷)C(O)R⁷, or —(C(R⁸)₂)ₚCR⁸═C(R⁸)₂; wherein said alkyl, cycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷;

R² is alkyl, cycloalkyl, heterocycloalkyl, alkenyl, cycloalkenyl, alkynyl, aryl, aralkyl, halogen, cyano, nitro, —OR⁷, —N(R⁷)₂, —S, —C(O)R⁷, —CO₂R⁷, —C(O)N(R⁷)₂, —OC(O)R⁷, —N(R⁷)C(O)R⁷, —SC(O)R⁷, or —(C(R⁸)₂)ₚCR⁸═C(R⁸)₂; wherein said alkyl, cycloalkyl, heterocycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷; or R² and R³ taken together form a 5-7 member ring containing 0, 1, or 2 heteroatoms selected from the group consisting of O and N; or R¹ and R² taken together form a 5-7 member ring containing 0, 1, or 2 heteroatoms selected from the group consisting of O and N; or R² is

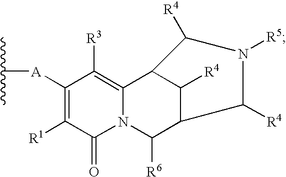

R⁴ represents independently for each occurrence H, alkyl, alkenyl, halogen, —OR⁷, —N(R⁷)₂, or —(C(R⁸)₂)ₚCR⁸═C(R⁸)₂;

R⁵ is H, alkyl, cycloalkyl, alkenyl, cycloalkenyl, alkynyl, aryl, aralkyl, —C(O)R⁷, —CO₂R⁷, —C(O)N(R⁷)₂, or —(C(R⁸)₂)ₚCR⁸═C(R⁸)₂; wherein said alkyl, cycloalkyl, alkenyl, cycloalkenyl, alkynyl, aryl, and aralkyl are optionally substituted with one or more of halogen, —OR⁷, —N(R⁷)₂, —SR⁷, —C(O)R⁷, —CO₂R⁷, —C(O)N(R⁷)₂, —OC(O)R⁷, or —N(R⁷)C(O)R⁷;

R⁷ represents independently for each occurrence H, alkyl, cycloalkyl, heterocycloalkyl, alkenyl, cycloalkenyl, allyl, alkynyl, aryl, aralkyl, alkoxyalkyl, aryloxyalkyl, or cycloalkyloxyalkyl;

R⁸ represents independently for each occurrence H or (C₁-C₆)alkyl;

A is an alkyl diradical, alkenyl diradical, aryl diradical, aralkyl diradical, or —(C(R⁸)₂)ₘ—X—(C(R⁸)₂)ₘ—;

X is O, —N(R⁷)—, or S;

m and p represent independently for each occurrence 1, 2, 3, 4, 5, or 6; and

n is 1 or 2.

In certain embodiments, the present invention relates to the aforementioned method, wherein R¹ represents independently for each occurrence H, alkyl, alkenyl, aryl, aralkyl, halogen, —C(O)R⁷, —CO₂R⁷, —C(O)N(R⁷)₂, or —(C(R⁸)₂)ₚCR⁸═C(R⁸)₂.

In certain embodiments, the present invention relates to the aforementioned method, wherein R¹ represents independently for each occurrence H or (C₁-C₆)alkyl.

In certain embodiments, the present invention relates to the aforementioned method, wherein R¹ represents independently for each occurrence H.

In certain embodiments, the present invention relates to the aforementioned method, wherein R² is alkyl, cycloalkyl, heterocycloalkyl, alkenyl, cycloalkenyl, alkynyl, aryl, aralkyl, halogen, cyano, nitro, —OR⁷, —N(R⁷)₂, —SR⁷, —C(O)R⁷, —CO₂R⁷, —C(O)N(R⁷)₂, —OC(O)R⁷, —N(R⁷)C(O)R⁷, or —(C(R⁸)₂)ₚCR⁸═C(R⁸)₂; wherein said alkyl, cycloalkyl, heterocycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷.

In certain embodiments, the present invention relates to the aforementioned method, wherein R² is alkyl, cycloalkyl, heterocycloalkyl, alkenyl, aryl, aralkyl, halogen, cyano, —C(O)R⁷, —CO₂R⁷, —C(O)N(R⁷)₂, or —(C(R⁸)₂)ₚCR⁸═C(R⁸)₂; wherein said alkyl, cycloalkyl, heterocycloalkyl, alkenyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷.

In certain embodiments, the present invention relates to the aforementioned method, wherein R² is alkyl, cycloalkyl, alkenyl, aryl, or —(C(R⁸)₂)ₚCR⁸═C(R⁸)₂; wherein said alkyl, cycloalkyl, alkenyl, and aryl are optionally substituted with one or more of halogen, —OR⁷, —N(R⁷)₂, or —SR⁷; wherein R⁷ is H or (C₁-C₆)alkyl.

In certain embodiments, the present invention relates to the aforementioned method, wherein R² is alkyl or cycloalkyl; wherein said alkyl and cycloalkyl are optionally substituted with one or more of halogen, —OR⁷, —N(R⁷)₂, or —SR⁷; wherein R⁷ is H or (C₁-C₆)alkyl.

In certain embodiments, the present invention relates to the aforementioned method, wherein R² is (C₁-C₆)alkyl optionally substituted with —OR⁷, —N(R⁷)₂, or —SR⁷; wherein R⁷ is H or (C₁-C₆)alkyl.

In certain embodiments, the present invention relates to the aforementioned method, wherein R² is (C₁-C₆)alkyl optionally substituted with —OR⁷; wherein R⁷ is H or (C₁-C₆)alkyl.

In certain embodiments, the present invention relates to the aforementioned method, wherein R² is (C₁-C₆)alkyl.

In certain embodiments, the present invention relates to the aforementioned method, wherein R² is methyl, ethyl, propyl, isopropyl, butyl, isobutyl, sec-butyl, or pentyl optionally substituted with —OR⁷; wherein R⁷ is H or (C₁-C₆)alkyl.

In certain embodiments, the present invention relates to the aforementioned method, wherein R² is methyl, ethyl, propyl, isopropyl, butyl, isobutyl, sec-butyl, or pentyl.

In certain embodiments, the present invention relates to the aforementioned method, wherein R² is methyl.

In certain embodiments, the present invention relates to the aforementioned method, wherein R² is —CH₂OH.

In certain embodiments, the present invention relates to the aforementioned method, wherein R³ represents independently for each occurrence H, alkyl, alkenyl, aryl, aralkyl, halogen, —C(O)R⁷, —CO₂R⁷, —C(O)N(R⁷)₂, or —(C(R⁸)₂)ₚCR⁸═C(R⁸)₂.

In certain embodiments, the present invention relates to the aforementioned method, wherein R³ represents independently for each occurrence H or (C₁-C₆)alkyl.

In certain embodiments, the present invention relates to the aforementioned method, wherein R³ represents independently for each occurrence H.

In certain embodiments, the present invention relates to the aforementioned method, wherein R⁴ represents independently for each occurrence H or alkyl.

In certain embodiments, the present invention relates to the aforementioned method, wherein R⁴ is H.

In certain embodiments, the present invention relates to the aforementioned method, wherein R⁵ is H, alkyl, cycloalkyl, aryl, aralkyl, —C(O)R⁷, —CO₂R⁷, —C(O)N(R⁷)₂, or —(C(R⁸)₂)ₚCR⁸═C(R⁸)₂.

In certain embodiments, the present invention relates to the aforementioned method, wherein R⁵ is H, alkyl, or benzyl.

In certain embodiments, the present invention relates to the aforementioned method, wherein R⁵ is H.

In certain embodiments, the present invention relates to the aforementioned method, wherein R⁶ represents independently for each occurrence H, alkyl, alkenyl, aryl, aralkyl, halogen, —C(O)R⁷, —CO₂R⁷, —C(O)N(R⁷)₂, or —(C(R⁸)₂)ₚCR⁸═C(R⁸)₂.

In certain embodiments, the present invention relates to the aforementioned method, wherein R⁶ represents independently for each occurrence H or (C₁-C₆)alkyl.

In certain embodiments, the present invention relates to the aforementioned method, wherein R⁶ represents independently for each occurrence H.

In certain embodiments, the present invention relates to the aforementioned method, wherein R⁷ represents independently for each occurrence H, alkyl, cycloalkyl, heterocycloalkyl, alkenyl, cycloalkenyl, allyl, alkynyl, aryl, or aralkyl.

In certain embodiments, the present invention relates to the aforementioned method, wherein R⁷ represents independently for each occurrence H, alkyl, cycloalkyl, aryl, or aralkyl.

In certain embodiments, the present invention relates to the aforementioned method, wherein R⁷ represents independently for each occurrence H, alkoxymethyl, aryloxymethyl, or cycloalkyloxymethyl.

In certain embodiments, the present invention relates to the aforementioned method, wherein R⁷ represents independently for each occurrence H or alkyl.

In certain embodiments, the present invention relates to the aforementioned method, wherein R⁷ is H.

In certain embodiments, the present invention relates to the aforementioned method, wherein n is 1.

In certain embodiments, the present invention relates to the aforementioned method, wherein R¹, R³, R⁴, R⁵, and R⁶ represent independently for each occurrence H or alkyl.

In certain embodiments, the present invention relates to the aforementioned method, wherein R¹, R³, R⁴, R⁵, and R⁶ represent independently for each occurrence H or alkyl; and n is 1.

In certain embodiments, the present invention relates to the aforementioned method, wherein R¹, R³, R⁴, R⁵, and R⁶ represent independently for each occurrence H or alkyl; n is 1; and R² is alkyl, cycloalkyl, heterocycloalkyl, alkenyl, aryl, aralkyl, halogen, cyano, —C(O)R⁷, —CO₂R⁷, —C(O)N(R⁷)₂, or —(C(R⁸)₂)ₚCR⁸═C(R⁸)₂; wherein said alkyl, cycloalkyl, heterocycloalkyl, alkenyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷.

In certain embodiments, the present invention relates to the aforementioned method, wherein R¹, R³, R⁴, R⁵, and R⁶ represent independently for each occurrence H or alkyl; n is 1; R² is alkyl, cycloalkyl, alkenyl, aryl, or —(C(R⁸)₂)ₚCR⁸═C(R⁸)₂; wherein said alkyl, cycloalkyl, alkenyl, and aryl are optionally substituted with one or more of halogen, —OR⁷, —N(R⁷)₂, or —SR⁷; and R⁷ is H or (C₁-C₆)alkyl.

In certain embodiments, the present invention relates to the aforementioned method, wherein R¹, R³, R⁴, R⁵, and R⁶ represent independently for each occurrence H or alkyl; n is 1; R² represents independently for each occurrence (C₁-C₆)alkyl optionally substituted with —OR⁷, —N(R⁷)₂, or —SR⁷; and R⁷ is H or (C₁-C₆)alkyl.

In certain embodiments, the present invention relates to the aforementioned method, wherein R¹, R³, R⁴, R⁵, and R⁶ are H; n is 1; R² represents independently for each occurrence methyl, ethyl, propyl, isopropyl, butyl, isobutyl, sec-butyl, or pentyl optionally substituted with —OR⁷; and R⁷ is H or (C₁-C₆)alkyl.

In certain embodiments, the present invention relates to the aforementioned method, wherein R¹, R³, R⁴, R⁵, and R⁶ are H; n is 1; and R² is methyl.

In certain embodiments, the present invention relates to the aforementioned method, wherein R¹, R³, R⁴, R⁵, and R⁶ are H; n is 1; and R² is —CH₂OH.

In certain embodiments, the present invention relates to the aforementioned method, wherein the α3β4/α4β2 nAChR subtype binding affinity ratio for said compound of formula I is greater than about 500:1 in a nAChR binding assay.

In certain embodiments, the present invention relates to the aforementioned method, wherein the α3β4/α4β2 nAChR subtype binding affinity ratio for said compound of formula I is greater than about 1000:1 in a nAChR binding assay.

In certain embodiments, the present invention relates to the aforementioned method, wherein the α3β4/α4β2 nAChR subtype binding affinity ratio for said compound of formula I is greater than about 2000:1 in a nAChR binding assay.

In certain embodiments, the present invention relates to the aforementioned method, wherein the α3β4/α4β2 nAChR subtype binding affinity ratio for said compound of formula I is greater than about 3000:1 in a nAChR binding assay.

In certain embodiments, the present invention relates to the aforementioned method, wherein said compound of formula I has a Kᵢ of less than about 500 nM in an assay based on an α4β2 nAChR receptor.

In certain embodiments, the present invention relates to the aforementioned method, wherein said compound of formula I has a Kᵢ of less than about 100 nM in an assay based on an α4β2 nAChR receptor.

In certain embodiments, the present invention relates to the aforementioned method, wherein said compound of formula I has a Kᵢ of less than about 50 nM in an assay based on an α4β2 nAChR receptor.

In certain embodiments, the present invention relates to the aforementioned method, wherein said compound of formula I has a Kᵢ of less than about 25 nM in an assay based on an α4β2 nAChR receptor.

In certain embodiments, the present invention relates to the aforementioned method, wherein said compound of formula I has a Kᵢ of less than about 10 nM in an assay based on an α4β2 nAChR receptor.

In certain embodiments, the present invention relates to the aforementioned method, wherein the mammal is a primate, equine, canine, or feline.

In certain embodiments, the present invention relates to the aforementioned method, wherein the mammal is a human.

In certain embodiments, the present invention relates to the aforementioned method, wherein the compound is administered orally.

In certain embodiments, the present invention relates to the aforementioned method, wherein the compound is administered intravenously.

In certain embodiments, the present invention relates to the aforementioned method, wherein the compound is administered sublingually.

In certain embodiments, the present invention relates to the aforementioned method, wherein the compound is administered ocularly.

In certain embodiments, the present invention relates to the aforementioned method, wherein the compound is administered transdermally.

In certain embodiments, the present invention relates to the aforementioned method, wherein the compound is administered rectally.

In certain embodiments, the present invention relates to the aforementioned method, wherein the compound is administered vaginally.

In certain embodiments, the present invention relates to the aforementioned method, wherein the compound is administered topically.

In certain embodiments, the present invention relates to the aforementioned method, wherein the compound is administered intramuscularly.

In certain embodiments, the present invention relates to the aforementioned method, wherein the compound is administered subcutaneously.

In certain embodiments, the present invention relates to the aforementioned method, wherein the compound is administered buccally.

In certain embodiments, the present invention relates to the aforementioned method, wherein the compound is administered nasally.

In certain embodiments, the present invention relates to the aforementioned method, wherein said mammal is suffering from Alzheimer's disease, Parkinson's disease, dyskinesias, Tourette's syndrome, schizophrenia, attention deficit disorder or tobacco abuse.

Another aspect of the invention relates to a method of treating a mammal suffering from Alzheimer's disease, Parkinson's disease, dyskinesias, Tourette's syndrome, schizophrenia, attention deficit disorder, anxiety, pain, depression, obsessive compulsive disorder, chemical substance abuse, alcoholism, memory deficit, pseudodementia, Ganser's syndrome, migraine pain, bulimia, obesity, premenstrual syndrome or late luteal phase syndrome, tobacco abuse, post-traumatic syndrome, social phobia, chronic fatigue syndrome, premature ejaculation, erectile difficulty, anorexia nervosa, disorders of sleep, autism, mutism, avoidance learning, or trichotillomania, comprising the step of administering to a mammal in need thereof a therapeutically effective amount of a compound of Formula II:

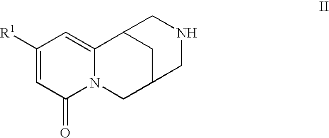

or a pharmaceutically acceptable salt thereof;

wherein

the stereochemical configuration at any stereocenter of said compound is R, S, or a mixture thereof;

R₁ is —OH, —SH, halogen, —CF₃, —CN, —NO₂, optionally substituted C₁-C₆ alkyl chain, optionally substituted benzyl, optionally substituted heteroaryl, optionally substituted cycloalkyl, —NH₂, di-[(C₁-C₆)alkylamino, (C₁-C₆) monoalkylamino, (C₆-C₁₀) arylamino, (C₃-C₈)cycloalkylamino, heteroarylamino, cycloheteroalkylamino; —C(O)R wherein R is H, optionally substituted (C₁-C₆)alkyl, optionally substituted aryl, or optionally substituted benzyl; —CO₂R wherein R is H, (C₁-C₆) alkyl, phenyl, or benzyl; —CON(R)₂ wherein each R is hydrogen, (C₁-C₆)alkyl or (C₆-C₁₀)aryl; —NHC(O)R, wherein R is optionally substituted alkyl (C₁-C₆ chain), optionally substituted aryl, or optionally substituted benzyl; —XR wherein X is O, S or N, and R is hydrogen, alkyl, or aryl bearing 0, 1 or 2 substituents; optionally benzene-fused (C₆-C₁₀) aryl; optionally benzene-fused (C₃-C₈)cycloalkyl; optionally benzene-fused heteroaryl wherein said heteroaryl group contains 5 to 10 atoms comprising one to four heteroatoms; optionally benzene-fused cycloheteroalkyl wherein said cycloheteroalkyl contains 4 to 8 atoms comprising one or two heteroatoms selected from group consisting of N, S and O; —CH₂XR, wherein X is O, S or N, and when X=O, R is selected from the group consisting of hydrogen, allyl, optionally substituted alkenyl, alkoxy methyl, cycloalkyloxy methyl, —C(O)R″ and aryl bearing 0, 1 or 2 substituents, wherein R″ is optionally substituted alkyl, optionally substituted aryl, or optionally substituted benzyl, when X=S or N, R is hydrogen, optionally substituted alkyl, optionally substituted aryl, —NH₂, di-[(C₁-C₆)alkylamino, (C₁-C₆)monoalkylamino, (C₆-C₁₀) arylamino, (C₃-C₈)cycloalkylamino, heteroarylamino, cycloheteroalkylamino or —NHC(O)R′″, wherein R′″ is optionally substituted (C₁-C₆)alkyl chain, optionally substituted aryl, optionally substituted benzyl; —(CH₂)ₙ—OCH₂-(10-Cytisine); —(CH₂)ₙ(10-Cytisine); alkenyl; alkynyl; wherein said alkenyl, alkynyl, and aryl are optionally substituted with halogen, CN, OH, hydroxymethyl, alkoxy, NO₂, amine, alkyl amine, or —NHC(O)R, wherein R is alkyl (C₁-C₆ chain), aryl, or benzyl; and n is 1, 2, 3, 4, 5, or 6.

In certain embodiments, the present invention relates to the aforementioned method, wherein said mammal is suffering from Alzheimer's disease, Parkinson's disease, dyskinesias, Tourette's syndrome, schizophrenia, attention deficit disorder or tobacco abuse.

Another aspect of the invention relates to a method of treating a mammal suffering from Alzheimer's disease, Parkinson's disease, dyskinesias, Tourette's syndrome, schizophrenia, attention deficit disorder, anxiety, pain, depression, obsessive compulsive disorder, chemical substance abuse, alcoholism, memory deficit, pseudodementia, Ganser's syndrome, migraine pain, bulimia, obesity, premenstrual syndrome or late luteal phase syndrome, tobacco abuse, post-traumatic syndrome, social phobia, chronic fatigue syndrome, premature ejaculation, erectile difficulty, anorexia nervosa, disorders of sleep, autism, mutism, avoidance learning, or trichotillomania, comprising the step of administering to a mammal in need thereof a therapeutically effective amount of a compound of Formula III:

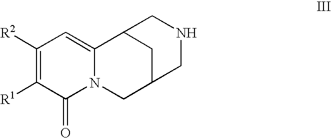

or a pharmaceutically acceptable salt thereof;

wherein

the stereochemical configuration at any stereocenter of said compound is R, S, or a mixture thereof;

R¹ and R² taken together form a 5-8 member ring containing 0, 1, 2, or 3 heteroatoms selected from the group consisting of N, O, and S; and said 5-8 member ring is optionally fused with an aryl or heteroaryl ring

In certain embodiments, the present invention relates to the aforementioned method, wherein said mammal is suffering from Alzheimer's disease, Parkinson's disease, dyskinesias, Tourette's syndrome, schizophrenia, attention deficit disorder or tobacco abuse.

### Kits of the Invention

Another aspect of the invention relates to kits for conveniently and effectively implementing the methods of this invention. Such kits comprise any subject composition, and a means for facilitating compliance with methods of this invention. Such kits provide a convenient and effective means for assuring that the subject to be treated takes the appropriate active in the correct dosage in the correct manner. The compliance means of such kits includes any means which facilitates administering the actives according to a method of this invention. Such compliance means include instructions, packaging, and dispensing means, and combinations thereof. Kit components may be packaged for either manual or partially or wholly automated practice of the foregoing methods. In other embodiments involving kits, this invention contemplates a kit including compositions of the present invention, and optionally instructions for their use.

### DEFINITIONS

For convenience, certain terms employed in the specification, examples, and appended claims are collected here.

The term “α3β4/α4β2 nAChR subtype binding affinity ratio” for a compound refers to the Kᵢ for the α3β4 receptor subtype divided by the Kᵢ for the α4β2 receptor subtype. For example, if the Kᵢ for the α3β4 receptor subtype is 20 nM while the Kᵢ for the α4β2 receptor subtype is 10 nM, then the compound has an α3β4/α4β2 nAChR subtype binding affinity ratio equal to 2.

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas:

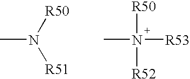

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula:

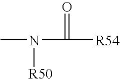

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

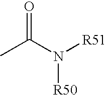

wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carboxyl” is art recognized and includes such moieties as may be represented by the general formulas:

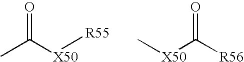

wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula:

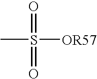

in which R57 is an electron pair, hydrogen, alkyl, cycloalkyl, or aryl.

The term “sulfate” is art recognized and includes a moiety that may be represented by the general formula:

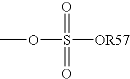

in which R57 is as defined above.

The term “sulfonamido” is art recognized and includes a moiety that may be represented by the general formula:

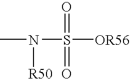

in which R50 and R56 are as defined above.

The term “sulfamoyl” is art-recognized and refers to a moiety that may be represented by the general formula:

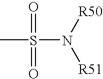

in which R50 and R51 are as defined above.

The term “sulfonyl” is art-recognized and refers to a moiety that may be represented by the general formula:

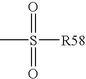

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

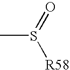

in which R58 is defined above.

The term “phosphoryl” is art-recognized and may in general be represented by the formula:

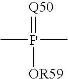

wherein Q50 represents S or O, and R59 represents hydrogen, a lower alkyl or an aryl. When used to substitute, e.g., an alkyl, the phosphoryl group of the phosphorylalkyl may be represented by the general formulas:

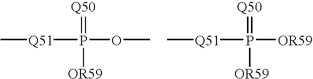

wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas:

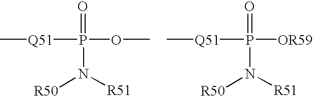

wherein Q51, R50, R51 and R59 are as defined above.

The term “phosphonamidite” is art-recognized and may be represented in the general formulas:

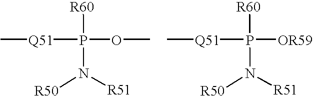

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

For purposes of this invention, the chemical elements are identified in accordance with the Periodic Table of the Elements, CAS version, Handbook of Chemistry and Physics, 67th Ed., 1986-87, inside cover.

### Pharmaceutical Compositions

In another aspect, the present invention provides pharmaceutically acceptable compositions which comprise a therapeutically-effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms. Preferred dosing is one administration per day.

While it is possible for a compound of the present invention to be administered alone, it is preferable to administer the compound as a pharmaceutical formulation (composition).

The compounds according to the invention may be formulated for administration in any convenient way for use in human or veterinary medicine, by analogy with other pharmaceuticals.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Ore., U.S.A., 1977).

### Micelles

Recently, the pharmaceutical industry introduced microemulsification technology to improve bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing absorption to the lymphatic system instead of the circulatory system which thereby bypasses the liver, and prevents destruction of the compounds in the hepatobiliary circulation.

In one aspect of invention, the formulations contain micelles formed from a compound of the present invention and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated, the presently preferred carriers are generally those that have Generally-Recognized-as-Safe (GRAS) status, and that can both solubilize the compound of the present invention and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Particularly preferred amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

### Polymers

Hydrophilic polymers suitable for use in the present invention are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. Preferred polymers are those having a molecular weight of from about 100 or 120 daltons up to about 5,000 or 10,000 daltons, and more preferably from about 300 daltons to about 5,000 daltons. In a particularly preferred embodiment, the polymer is polyethyleneglycol having a molecular weight of from about 100 to about 5,000 daltons, and more preferably having a molecular weight of from about 300 to about 5,000 daltons. In a particularly preferred embodiment, the polymer is polyethyleneglycol of 750 daltons (PEG(750)). Polymers may also be defined by the number of monomers therein; a preferred embodiment of the present invention utilizes polymers of at least about three monomers, such PEG polymers consisting of three monomers (approximately 150 daltons).

Other hydrophilic polymers which may be suitable for use in the present invention include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

### Cyclodextrins

Cyclodextrins are cyclic oligosaccharides, consisting of 6, 7 or 8 glucose units, designated by the Greek letter alpha, beta or gamma, respectively. Cyclodextrins with fewer than six glucose units are not known to exist. The glucose units are linked by alpha-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17beta-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see: Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble crosslinked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

### Liposomes

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm Oligolamellar large vesicles and multilamellar vesicles have multiple, usually concentric, membrane layers and are typically larger than 0.1 μm Liposomes with several nonconcentric membranes, i.e., several smaller vesicles contained within a larger vesicle, are termed multivesicular vesicles.

One aspect of the present invention relates to formulations comprising liposomes containing a compound of the present invention, where the liposome membrane is formulated to provide a liposome with increased carrying capacity. Alternatively or in addition, the compound of the present invention may be contained within, or adsorbed onto, the liposome bilayer of the liposome. The compound of the present invention may be aggregated with a lipid surfactant and carried within the liposome's internal space; in these cases, the liposome membrane is formulated to resist the disruptive effects of the active agent-surfactant aggregate.

According to one embodiment of the present invention, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes of the present invention are in solubilized form. Aggregates of surfactant and active agent (such as emulsions or micelles containing the active agent of interest) may be entrapped within the interior space of liposomes according to the present invention. A surfactant acts to disperse and solubilize the active agent, and may be selected from any suitable aliphatic, cycloaliphatic or aromatic surfactant, including but not limited to biocompatible lysophosphatidylcholines (LPCs) of varying chain lengths (for example, from about C.sub.14 to about C.sub.20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMCs in the micromolar range; higher CMC surfactants may be utilized to prepare micelles entrapped within liposomes of the present invention, however, micelle surfactant monomers could affect liposome bilayer stability and would be a factor in designing a liposome of a desired stability.

Liposomes according to the present invention may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic DD, Liposomes from physics to applications, Elsevier Science Publishers BV, Amsterdam, 1993.

For example, liposomes of the present invention may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present invention, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988).

### Release Modifiers

The release characteristics of a formulation of the present invention depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In all cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range should be between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups, such as cyanoacrylates and methacrylates).

### EXEMPLIFICATION

The invention now being generally described, it will be more readily understood by reference to the following examples, which are included merely for purposes of illustration of certain aspects and embodiments of the present invention, and are not intended to limit the invention.

### Example 1

General Chemistry Methods: All solvents and reagents were used as obtained from commercial sources unless otherwise indicated. All starting materials were also obtained from commercial source. All reactions were performed under argon unless otherwise noted. Organic layers were washed with water, brine, dried over anhydrous Na₂SO₄ and evaporated at 40° C. under reduced pressure (standard work up). ¹H and ¹³C NMR spectra were recorded on an Avance 400 Bruker instrument operating at 400 MHz for ¹H and 100 MHz for ¹³C. Deuterated chloroform (99.8% D) or methanol (99.8% D) was used as solvents. ¹H Chemical shifts value (δ), from tetramethylsilane as internal standard. ¹³C chemical shifts (δ) are referenced to CDCl₃ (central peak, δ=77.00 ppm) and CD₃OD (central peak, δ=49.15 ppm) as the internal standard. Mass spectra were measured in positive mode electrospray ionization (ESI). The HRMS data were obtained on a Micromass Q-TOF-2™ instrument. TLC was performed on silica gel 60 F₂₅₄ glass plates; column chromatography was performed using silica gel (35-75 mesh). All final compounds send for biological assay are further purified by HPLC. Analytical HPLC was performed using a Shimadzu LC-10AD system, equipped with a Waters 484 tunable absorbance detector set at 254, 280, 310 or 360 nm.

### Synthetic Procedures and Experimental Details for Compounds 6 and 7.

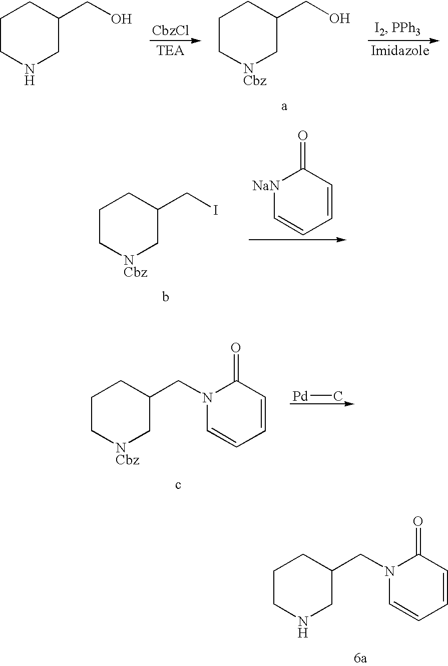

3-Hydroxymethylpiperidine-1-carboxylic acid benzyl ester (a): To a stirred solution of 3-hydroxymethylpiperidine (1 g, 8.7 mmol) in CH₂Cl₂ (50 mL) and Et₃N (1.12 mml) at 0° C. was added dropwise CbzCl (1.24 mL, 8.7 mmol). The reaction mixture was stirred at room temperature overnight. The mixture was washed with brine, dried over Na₂SO₄, and concentrated. The residue was purified by chromatography with hexane-EtOAc (2:1) to give colorless oil (2 g, 94%). ¹H NMR (CDCl₃, 400 MHz) δ 7.35 (m, 5H), 5.12 (m, 2H), 4.20-3.60 (m, 2H), 3.47 (m, 2H), 3.20-2.20 (m, 3H), 1.82-1.10 (m, 5H). ¹³C NMR (CDCl₃, 100 MHz) δ 155.55, 136.73, 128.38, 127.85, 127.69, 66.96, 64.31, 46.74, 44.71, 38.01, 26.77, 24.05.

3-Iodomethylpiperidine-1-carboxylic acid benzyl ester (b): To a stirred solution of PPh₃ (3.5 g) in dry CH₂Cl₂ (60 mL) was added I₂ (3.34 g) under N₂. After stirred for 15 min, imidazole (1.03 g) was added in one portion, followed by addition of 3-hydroxymethylpiperidine-1-carboxylic acid benzyl ester (1.5 g, 6.02 mmol) in CH₂Cl₂ (5 mL). The reaction mixture was stirred at room temperature for 4 h, washed with 5% aqueous NaHSO₃ and brine, dried, and concentrated. The residue was purified by chromatography with hexane-EtOAc (4:1) to give viscous oil (2.1 g, 97%). ¹H NMR (CDCl₃, 400 MHz) δ 7.35 (m, 5H), 5.13 (s, 2H), 4.15 (br s, 1H), 3.96 (dt, 1H, J=13.2, 3.9 Hz), 3.07 (d, 2H, J=6.3 Hz), 2.85 (m, 1H), 2.66 (br s, 1H), 1.94 (m, 1H), 1.74-1.38 (m, 3H), 1.33-1.17 (m, 1H). ¹³C NMR (CDCl₃, 100 MHz) δ 155.10, 136.71, 128.40, 127.89, 127.75, 67.02, 49.71, 44.31, 37.90, 31.21, 24.18, 9.52.

3-(2-Oxo-2H-pyridin-1-ylmethyl)piperidine-1-carboxylic acid benzyl ester (c): To a stirred solution of 2-hydroxypyridine (200 mg, 2 mmol) in DMF (5 mL) was added NaH (60% mixture in mineral oil, 100 mg, 2.5 mmol). The mixture was stirred at 80° C. under N₂ for 2 h, and then 3-iodomethylpiperidine-1-carboxylic acid benzyl ester (720 mg, 2 mmol) was added. The mixture was stirred at 80° C. for 10 h, cooled to room temperature, quenched with water, and extracted with EtOAc. The combined organic layers were washed with brine, dried, and concentrated. The residue was purified by chromatography with CH₂Cl₂-EtOAc-MeOH (10:10:1) to give viscous oil (475 mg, 73%). ¹H NMR (CDCl₃, 400 MHz) δ 7.30 (m, 7H), 6.55 (d, 1H, J=9.0 Hz), 6.12 (m, 1H), 5.11 (s, 2H), 4.10-3.50 (m, 4H), 3.09 (t, 1H, J=10.2 Hz), 2.92 (dd, 1H, J=13.2, 9.0 Hz), 2.10 (m, 1H), 1.85-1.20 (m, 4H).

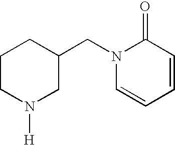

1-(Piperidin-3-ylmethyl)pyridin-2(1H)-one (6a): A mixture of 3-(2-oxo-2H-pyridin-1-ylmethyl)piperidine-1-carboxylic acid benzyl ester (100 mg) and 5% Pd—C (20 mg) in EtOH (15 mL) was stirred under H₂ (1 atm). The reaction was traced by TLC. The catalyst was filtered and the filtration was concentrated and purified by chromatography with CH₂Cl₂:MeOH:NH₃.H₂O (10:1:0.1) to give a syrup (50 mg, 85%). ¹H NMR (CDCl₃, 400 MHz) δ 7.31 (m, 1H), 7.21 (dd, 1H, J=6.9, 2.1 Hz), 6.56 (d, 1H, J=9.0 Hz), 6.14 (t, 1H, J=6.6 Hz), 3.90 (dd, 1H, J=13.0, 8.1 Hz), 3.76 (dd, 1H, J=13.0, 6.7 Hz), 2.98 (m, 2H), 2.62 (t, 1H, J=9.8 Hz), 2.44 (t, 1H, J=11.2 Hz), 2.10-1.10 (m, 6H).

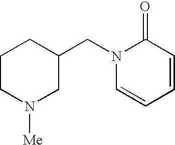

1-[(1-Methylpiperidin-3-yl)methyl]pyridin-2(1H)-one (6b): ¹H NMR (CDCl₃, 400 MHz): δ 7.54 (t, 1H, J=7.3 Hz), 7.44 (d, 1H, J=6.1 Hz), 6.77 (d, 1H, J=8.9 Hz), 6.48 (t, 1H, J=6.6 Hz), 4.22 (dd, 1H, J=4.4, 8.7 Hz), 3.88 (dd, 1H, J=5.2, 8.1 Hz), 3.59 (d, 1H, J=11.4 Hz), 3.45 (d, 1H, J=11.3 Hz), 2.81 (s, 3H), 2.76-2.64 (m, 2H), 2.57 (br s, 1H), 1.99 (br s, 2H), 1.91 (d, 1H, J=12.9 Hz), 1.37-1.26 (m, 1H). ¹³C NMR (CDCl₃, 100 MHz): δ 141.8, 137.7, 119.7, 109.5, 57.4, 54.9, 51.3, 44.1, 35.6, 25.6, 22.3.

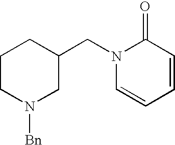

1-[(1-Benzylpiperidin-3-yl)methyl]pyridin-2(1H)-one (6c): ¹H NMR (CD₃OD, 400 MHz): δ 7.48-7.44 (m, 2H), 7.29-7.22 (m, 5H), 6.49 (d, 1H, J=8.8 Hz), 6.30-6.27 (m, 1H), 3.88 (d, 2H, J=6.7 Hz), 3.48 (d, 2H, J=4.8 Hz), 2.71 (dd, 2H, J=10.2, 15.3 Hz), 2.17-2.08 (m, 2H), 1.93-1.88 (m, 1H), 1.74-1.64 (m, 2H), 1.59-1.49 (m, 1H), 1.16-1.08 (m, 1H). ¹³C NMR (CD₃OD, 100 MHz): δ 164.9, 141.9, 140.3, 138.4, 130.8, 129.3, 128.4, 120.7, 108.3, 64.4, 57.7, 55.0, 54.1, 36.8, 28.9, 25.2.

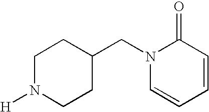

1-(Piperidin-4-ylmethyl)pyridin-2(1H)-one (7a): ¹H NMR (CDCl₃, 400 MHz): δ 9.26 (br s, 1H), 7.53 (t, 1H, J=7.3 Hz), 7.34 (d, 1H, J=5.8 Hz), 6.79 (d, 1H, J=9.0 Hz), 6.41 (t, 1H, J=6.6 Hz), 3.94 (d, 2H, J=7.0 Hz), 3.48 (d, 2H, J=11.7 Hz), 2.91 (d, 2H, J=9.4 Hz), 2.33 (br s, 1H), 1.86 (d, 2H, J=13.2 Hz), 1.73-1.65 (m, 2H); ¹³C NMR (CDCl₃, 100 MHz): δ 141.3, 138.2, 120.4, 108.3, 55.6, 43.7, 32.8, 26.0.

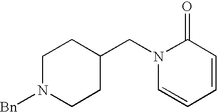

1-[(1-Benzyl-piperidin-4-yl)methyl]pyridin-2(1H)-one (7b): ¹H NMR (CDCl₃, 400 MHz): δ 7.57 (t, 1H, J=7.4 Hz), 7.48-7.41 (m, 6H), 6.84 (d, 1H, J=8.9 Hz), 6.54-6.47 (m, 1H), 4.25-4.19 (m, 2H), 4.03-3.98 (m, 1H), 3.75-3.70 (m, 1H), 3.51-3.48 (m, 1H), 3.30-3.29 (m, 1H), 3.12-3.01 (m, 1H), 2.93-2.90 (m, 1H), 2.64-2.57 (m, 1H), 2.39-2.25 (m, 1H), 2.01-1.96 (m, 2H), 1.83-1.78 (m, 1H). ¹³C NMR (CDCl₃, 400 MHz): δ 141.8, 137.6, 130.2, 130.1, 129.5, 129.4, 109.7, 58.9, 56.8, 53.8, 52.3, 50.3, 48.4, 34.7, 34.1, 33.9, 32.0, 30.2, 28.9.

TABLE A
Primary screening of 6a-c and 7a-b.

| | | Estimated Kᵢ (nM)* | | | | | | |
|---|---|---|---|---|---|---|---|---|
| Ligand | Concentration | α2β2 | α2β4 | α3β2 | α3β4 | α4β2 | α4β4 | α6β42 |
| 6a | 10 μM | 4,630 | 159,000 | 18,700 | 535,000 | 1,780 | 5,550 | 253,000 |
| 6b | 10 μM | 19,000 | 180,000 | 6,000 | 160,000 | 980 | 2,600 | 76,000 |
| 6c | 10 μM | 100,000 | 91,000 | 4,300 | 83,000 | 540 | 1,400 | 54,000 |
| 7a | 10 μM | 3,600 | 80,000 | 12,000 | 750,000 | 1,600 | 5,400 | 87,000 |
| 7b | 10 μM | 26,000 | 440,000 | 7,300 | 620,000 | 1,300 | 4,500 | 470,000 |

*The Kᵢ values are estimated from the single concentration competition binding assays.

### Example 2

### Representative Synthetic Scheme for Compounds 10 Through 17

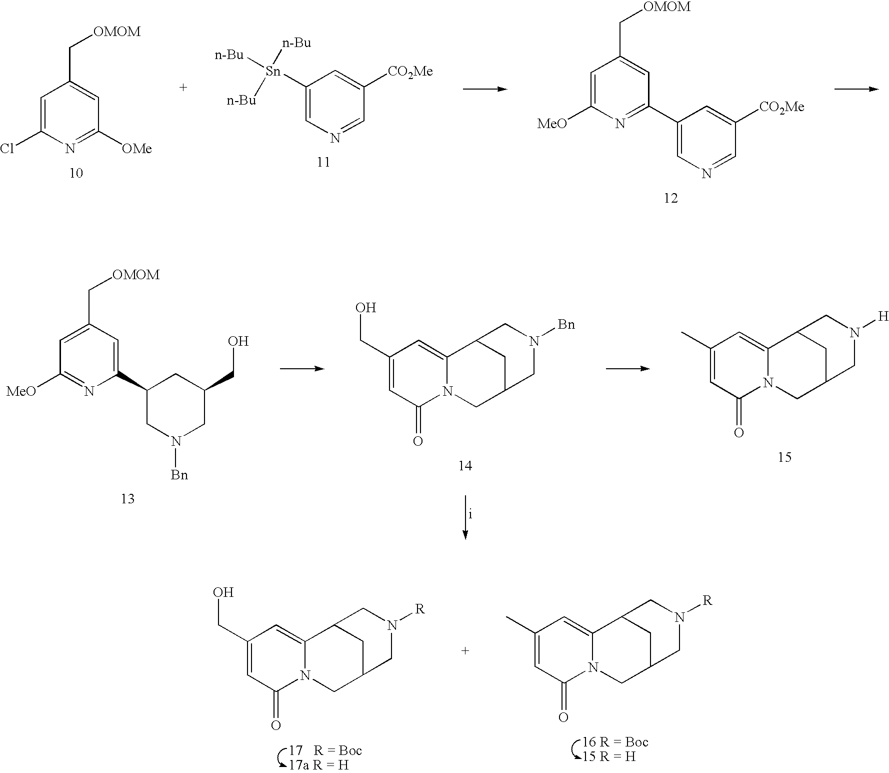

### Synthetic Procedures and Experimental Details

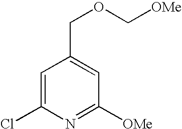

2-Chloro-6-methoxy-4-[(methoxymethoxy)methyl]pyridine (10). Boron trifluoride etherate (9.32 mL, 75.81 mmol) was added dropwise under argon during 15 min at 0° C. to a solution of dimethoxymethane (38.22 mL, 431.5 mmol) and (2-chloro-6-methoxy-pyridin-4-yl)-methanol (9), (10.700 g, 61.64 mmol) in dry dichloromethane (80 mL). After the addition, the reaction mixture was stirred at room temperature for 4 h, cooled to 0° C. and quenched by dropwise addition of water. Diluted with dichloromethane and the organic layer was washed with saturated sodium bicarbonate and brine, dried over anhydrous sodium sulfate and evaporated. The crude product was purified using silica gel column chromatography (10% EtOAc/Hexane) to afford 12.820 g, (95%) of the protected alcohol 10. ¹H NMR (CDCl₃, 400 MHz): δ 6.91 (s, 1H), 6.65 (d, 1H, J=0.7 Hz), 4.72 (s, 2H), 4.54 (s, 2H), 3.95 (s, 3H), 3.42 (s, 3H). ¹³C NMR (CDCl₃, 100 MHz): 163.7, 152.3, 148.1, 114.1, 106.5, 95.7, 66.5, 55.2, 53.7.

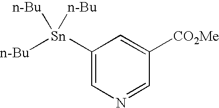

Methyl-5-(tri-n-butyl)stannylnicotinate (11): The procedure reported by O'Neill was used with modifications (Org. Lett. 2000, 2, 4201-4204). To an oven dried 500 mL 3-necked round bottom flask was added 18 g (83.32 mmol) methyl-5-bromonicotinate, 41.74 mL (48.33 g, 83.32 mmol) hexabutyldistannane and 180 mL anhydrous DMF under argon. After three vacuum/argon cycles, 3.15 g (4.16 mmol) benzyl bis(triphenylphosphine) palladium (II) chloride was added followed by two additional vacuum/argon cycles. The reaction mixture was heated in a pre heated oil bath at 130° C. for 5 h. and cooled to room temperature. The reaction mixture was filtered through celite and the filtrate was diluted with ethyl acetate and brine. The solution was adjusted to pH 8 with saturated sodium bicarbonate solution. The organic phase was separated and the aqueous phase washed several times with ethyl acetate. After standard work up and evaporation, the residue was partitioned between acetonitrile/pentane to remove the stannane byproducts. The acetonitrile phase was concentrated to obtain the crude product which was purified further using a silica gel column chromatography. The product got eluted with 9:1 hexane/ethyl acetate mixture which upon evaporation of the solvent gave 12.8 g (38%) of the stannyl derivative as light yellow oil. ¹H NMR (CDCl₃, 400 MHz): δ 9.11 (s, 1H), 8.73 (s, 1H), 8.34 (s, 1H), 3.97 (s, 3H), 1.90-1.52 (m, 6H), 1.37-1.31 (m, 6H), 1.16-1.12 (m, 6H), 0.89 (t, 9H, J=7.3 Hz).

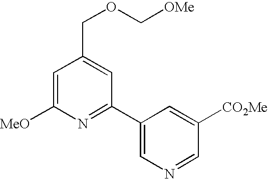

6-Methoxy-4-[(methoxymethoxy)methyl]-[2,3]bipyridinyl-5′-carboxylic acid methyl ester (12). A mixture of Methyl-5-(tri-n-butyl)stannylnicotinate (11.210 g, 26.30 mmol) and 10 (5.725 g, 26.30 mmol) were dissolved in dry DMF (92 mL) in a 500 ml three necked round bottom flask. After three vacuum/argon cycles, tetrakis(triphenylphosphine)palladium (0) (3.040 g, 2.63 mmol) was added to the stirring reaction mixture under argon. After one additional vacuum/argon cycle, the reaction mixture was stirred in a preheated oil bath at 130° C. and stirred overnight (18 h). The reaction mixture was cooled to room temperature and filtered through a short celite pad. The filtrate was partitioned between ethyl acetate and brine. The solution was adjusted to pH 8 with saturated bicarbonate solution. The organic phase was extracted with ethyl acetate, washed with brine, dried and concentrated. The crude product was partitioned between acetonitrile and pentane, the acetonitrile phase was evaporated in vacuo. Pure product was obtained after passing the crude mixture through a silica gel column and eluted using 40% EtOAc in hexane, as light yellow oil which turned solid upon cooling (6.750 g, 80%). ¹H NMR (CDCl₃, 400 MHz): δ 9.44 (s, 1H), 9.21 (s, 1H), 8.85 (s, 1H), 7.39 (s, 1H), 6.76 (s, 1H), 4.75 (s, 2H), 4.63 (s, 2H), 4.04 (s, 3H), 3.99 (s, 3H), 3.43 (s, 3H). ¹³C NMR (CDCl₃, 100 MHz): δ 165.8, 164.5, 151.9, 151.1, 151.0, 151.5, 134.8, 134.2, 125.8, 111.8, 108.6, 96.1, 67.4, 55.5, 53.5, 52.5.

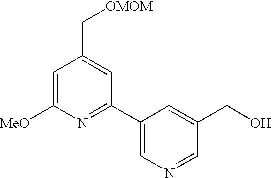

(6-Methoxy-4-[(methoxymethoxy)methyl]-[2,3]bipyridinyl-5′-yl)-methanol (12a): To a solution of the methyl ester 12 (6.500 g, 20.42 mmol) in anhydrous THF (300 mL) at −20 to −25° C. under argon, was added drop wise 21.4 mL (21.44 mmol) of 1M solution of lithium aluminium hydride in THF. During the addition the color of the reaction mixture turned brown. The reaction mixture kept stirring at −20 to −25° C. for 3.5 h. The reaction was quenched by slow addition of saturated NH₄Cl solution, extracted with ethyl acetate, washed with brine, dried and evaporated to remove the solvent. Silica gel column chromatography of the crude product using 3% MeOH/EtOAc mixture afforded 3.495 g (59%) of the alcohol 12a. ¹H NMR (CDCl₃, 400 MHz): δ 9.09 (d, 1H, J=1.9 Hz), 8.52 (d, 1H, J=1.8 Hz), 8.33 (d, 1H, J=1.9 Hz), 7.30 (s, 1H), 6.71 (d, 1H, J=0.6 Hz), 4.79 (s, 2H), 4.73 (s, 2H), 4.59 (s, 2H), 3.99 (s, 3H), 3.41 (s, 3H). ¹³C NMR (CDCl₃, 100 MHz): δ 164.5, 151.9, 151.1, 148.3, 147.2, 136.8, 134.5, 133.1, 111.8, 108.3, 96.2, 67.6, 62.5, 55.7, 53.6.

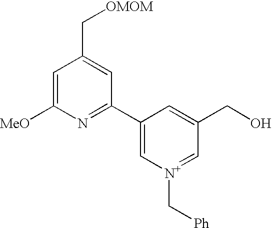

1′-Benzyl-5′-hydroxymethyl-6-methoxy-4-[(methoxymethoxy)methyl]-[2,3]bipyridinyl-1′-ium bromide (12b): To a solution of the alcohol 12a (1.34 g, 4.61 mmol) in 50 mL of dry acetonitrile was added benzyl bromide (0.97 mL, 5.54 mmol). The reaction mixture was heated under reflux for 1 h 45 min and cooled to room temperature. TLC showed complete conversion of the starting material. The solvent was removed under vacuum and the resulting sticky brown crude mass rinsed with hexane. ¹H NMR showed the presence of trace amount of benzyl bromide as impurity. The desired material obtained (1.82 g) was used as such for the next hydrogenation step. ¹H NMR (CDCl₃, 300 MHz): δ 9.99 (s, 1H), 9.69 (s, 1H), 9.38 (s, 1H), 7.80 (s, 1H), 7.37-7.29 (m, 5H), 6.79 (s, 1H), 6.17 (s, 2H), 5.61 (t, 1H, J=5.7 Hz), 4.94 (d, 2H, J=5.3 Hz), 4.71 (s, 2H), 4.62 (s, 2H), 3.91 (s, 3H), 3.37 (s, 3H).

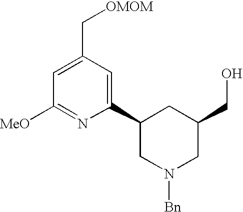

(1′-Benzyl-6-methoxy-4-[(methoxymethoxy)methyl]-1′,2′,3′,4′,5′,6′-hexahydro-[2,3]bipyridinyl-5′-yl)-methanol (13): To a solution of 1.816 g (3.935 mmol) of the salt 12b in 180 mL of methanol and 1.106 mL (7.87 mmol) triethylamine, was added 180 mg PtO₂ in a Parr hydrogenation bottle. After purged of all oxygen by three vacuum/hydrogen cycles, the reaction mixture was agitated in a Parr Apparatus under 55 psi hydrogen pressures for 3 h. (Same result obtained when the reaction mixture kept stirring at 1 atm hydrogen pressure at room temperature overnight). The catalyst was removed by filtration through a short celite pad and the filtrate was evaporated. The residue was diluted with ethyl acetate and saturated sodium bicarbonate solution. The organic phase was washed with saturated brine, dried and evaporated to get a crude mass as 5:1 mixture of cis and trans isomers (based on ¹H NMR of the crude sample). The crude product was purified by silica gel column chromatography using DCM/MeOH (98:2) to afford the more polar cis isomer 13 (1.02 g, 67%). ¹H NMR (CDCl₃, 400 MHz): δ 7.33-7.26 (m, 5H), 6.70 (s, 1H), 6.56 (s, 1H), 4.71 (s, 2H), 4.52 (s, 2H), 3.92 (s, 3H), 3.61 (s, 2H), 3.57-3.50 (m, 2H), 3.42 (s, 3H), 3.13-3.10 (m, 2H), 2.99 (t, 1H, J=11.2 Hz), 2.20-2.15 (m, 1H), 2.01 (d, 2H, J=10.9 Hz), 1.78 (t, 1H, J=10.9 Hz), 1.37 (q, 1H, J=12.4 Hz). ¹³C NMR (CDCl₃, 100 MHz): δ 163.8, 161.1, 150.0, 137.9, 129.2, 128.1, 126.9, 112.6, 105.6, 95.9, 67.5, 66.0, 63.3, 59.0, 56.7, 55.3, 53.2, 43.6, 38.9, 33.1.

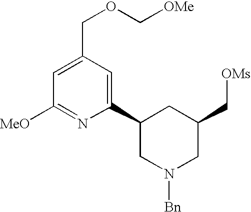

Methanesulfonic acid 1′-benzyl-6-methoxy-4-[(methoxymethoxy)methyl]-1′,2′,3′,4′,5′,6′-hexahydro-[2,3]bipyridinyl-5′-ylmethyl ester (13a): To a stirred solution of 13 (140 mg, 0.36 mmol) in dry dichloromethane (7 mL) at 0° C. under argon was added triethylamine (0.102 mL, 0.73 mmol) and mesyl chloride (0.042 mL, 0.54 mmol). After 30 min, diluted with dichloromethane and extracted with water. After standard work up, the crude residue was purified by flash silica gel column chromatography using 2% methanol in dichloromethane as solvent to afford the mesylate intermediate 13a (142 mg, 84%) as a light yellow liquid. ¹H NMR (CDCl₃, 400 MHz): δ 7.32-7.21 (m, 5H), 6.67 (s, 1H), 6.54 (s, 1H), 4.68 (s, 2H), 4.49 (s, 2H), 4.13-4.04 (m, 2H), 3.88 (s, 3H), 3.57 (s, 2H), 3.37 (s, 3H), 3.08-3.02 (m, 2H), 2.95 (s, 3H), 2.25 (br s, 1H), 2.01 (d, 2H, J=9.7 Hz), 1.82 (t, 1H, J=10.9 Hz), 1.43 (q, 1H, J=12.4 Hz), 1.24 (t, 1H, J=7.1 Hz). ¹³C NMR (CDCl₃, 100 MHz): δ 163.8, 160.4, 150.1, 137.9, 128.8, 128.1, 126.9, 112.6, 105.8, 95.8, 72.3, 67.4, 62.9, 60.1, 58.5, 55.7, 55.3, 53.1, 43.4, 37.0, 36.1, 32.5.

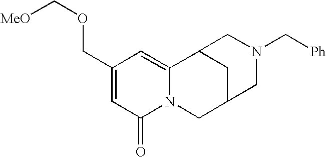

3-Benzyl-10-[(methoxymethoxy)methyl]-1,2,3,4,5,6-hexahydro-1,5-methano-pyrido[1,2-a][1,5]diazocin-8-one (13b): A solution of the above mesylate 13a, (759 mg, 1.64 mmol) in anhydrous toluene (100 mL) was refluxed for 3 h under argon atmosphere. The reaction mixture was cooled to room temperature and evaporated to get 480 mg (83%) of N-benzyl cytisine derivative (97% purity by HPLC). This is used as such for the next step. ¹H NMR (CDCl₃, 400 MHz): δ 7.17-7.16 (m, 3H), 6.98 (d, 2H, J=5.96 Hz), 6.48 (s, 1H), 5.92 (s, 1H), 4.68 (s, 2H), 4.40 (s, 2H), 4.07 (d, 1H, J=15.2 Hz), 3.85 (dd, 1H, J=6.6, 8.6 Hz), 3.42 (s, 2H), 3.40 (s, 3H), 2.93 (br s, 2H), 2.86 (d, 1H, J=9.5 Hz) 2.40-2.31 (m, 3H), 1.89 (d, 1H, J=12.2 Hz), 1.78 (d, 1H, J=11.9 Hz). ¹³C NMR (CDCl₃, 100 MHz): δ 162.8, 150.7, 149.2, 137.5, 128.4, 127.6, 127.5, 126.3, 112.4, 102.8, 95.3, 66.5, 61.3, 59.5, 59.2, 55.1, 49.3, 35.7, 27.5, 25.3.

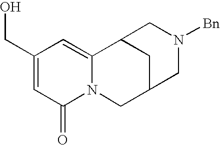

3-Benzyl-10-(hydroxymethyl)-1,2,3,4,5,6-hexahydro-1,5-methano-pyrido[1,2-a][1,5]diazocin-8-one (14): A mixture of the above cytisine derivative 13b (674 mg, 1.90 mmol) and trifluoroacteic acid (10 mL) was stirred at room temperature for 4 h. Removed the excess TFA under vacuo, and basified the crude reaction mixture with aqueous ammonia, diluted with ethyl acetate and then washed with saturated aqueous NaHCO₃, stirred for 2 h. Extracted with ethyl acetate and the organic layer was dried and concentrated. The crude sticky brown mass was purified by silica gel column chromatography using DCM/MeOH/NH₃ (95:5:1) mixture. Concentrated and dried under vacuo to afford 540 mg (91%) of the pure N-benzyl cytisine derivative 14. ¹H NMR (CDCl₃, 400 MHz): δ 7.2-7.15 (m, 3H), 6.99 (d, 2H, J=6.3 Hz), 6.51 (s, 1H), 5.98 (s, 1H), 4.53 (s, 2H), 4.08 (d, 1H, J=15.2 Hz), 3.87 (dd, 1H, J=8.7, 6.5 Hz), 3.49 (s, 1H), 3.42 (s, 2H), 2.94 (s, 2H), 2.87 (d, 1H, J=10.5 Hz), 2.41 (s, 1H), 2.34 (d, 2H, J=8.7 Hz), 1.90 (d, 1H, J=12.6 Hz), 1.79 (d, 1H, J=12.5 Hz). ¹³C NMR (CDCl₃, 100 MHz): δ 163.9, 153.9, 150.8, 137.9, 128.2, 128.1, 126.9, 112.1, 103.8, 62.9, 61.8, 60.0, 59.6, 49.9, 35.5, 27.9, 25.9.

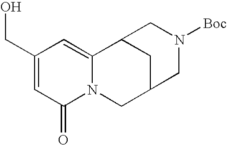

10-(Hydroxymethyl)-8-oxo-1,5,6,8-tetrahydro-2H,4H-1,5-methano-pyrido[1,2-a][1,5]diazocine-3-carboxylic acid tert-butyl ester (17): To a mixture of the above N-Benzyl cytisine derivative 14, (28 mg, 0.09 mmol) and Boc-anhydride (39 mg, 0.18 mmol) in degassed MeOH was added 6.8 mg of 20% Pd(OH)₂—C, degassed three times and refluxed the reaction mixture under 1 atm H₂ pressure for 30 min. cooled. TLC showed complete conversion of the starting material and two extra spots under UV light. The catalyst was removed by filtration, washed with MeOH and concentrated. After usual aqueous work up with EtOAc and evaporation of the solvent, the products were separated using preparative HPLC (CH₃CN/H₂O mixture in 0.05% TFA) to get 11 mg of 10-hydroxy methyl (17) and 15 mg of 10-methyl cytisine (16) derivatives respectively.

Alternative procedure: When the hydrogenation under the above conditions was stopped after 5′ min reflux, exclusive formation of the 10-hydroxy methyl cytisine derivative (17) obtained (97%). ¹H NMR (CDCl₃, 400 MHz): δ 6.42 (s, 1H), 6.12 (s, 1H), 4.50 (s, 2H), 4.23-4.13 (m, 3H), 3.80 (dd, 1H, J=6.5, 9.2 Hz), 3.09-2.96 (m, 3H), 2.41 (br s, 1H), 2.01-1.92 (m, 2H), 1.32-1.21 (m, 9H).

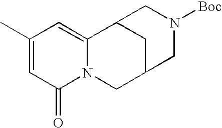

10-methyl-8-oxo-1,5,6,8-tetrahydro-2H,4H-1,5-methano-pyrido[1,2-a][1,5]diazocine-3-carboxylic acid tert-butyl ester (16): ¹H NMR (CDCl₃, 400 MHz): δ 6.68 (s, 1H), 6.29 (s, 1H), 4.30-4.14 (m, 3H), 3.94 (dd, 1H, J=6.3, 9.3 Hz), 3.10 (br s, 3H), 2.48 (s, 1H), 2.28 (s, 3H), 2.02 (br s, 2H), 1.34-1.24 (m, 9H).

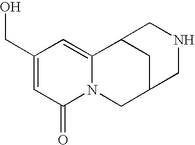

10-(Hydroxymethyl)-1,2,3,4,5,6-hexahydro-1,5-methano-pyrido[1,2-a][1,5]diazocin-8-one (17a): The above 10-hydroxymethyl cytisine derivative 17, (28 mg, 0.09 mmol) in a mixture of TFA/DCM (0.08/0.8) was stirred at room temperature for 2 h. Removed the excess TFA under vacuo, and basified the crude reaction mixture with aqueous ammonia, diluted with ethyl acetate and extracted. The organic layer was dried and concentrated. The crude mass was purified using preparative HPLC (CH₃CN/H₂O mixture in 0.05% TFA). Concentrated and dried under vacuo to afford 18 mg (93%) of the pure 10-hydroxymethyl cytisine 17a. ¹H NMR (MeOD, 400 MHz): δ 6.40 (s, 1H), 6.26 (d, 1H, J=1.3 Hz), 4.38 (s, 2H), 4.06 (d, 1H, J=15.8 Hz), 3.86 (dd, 1H, J=8.8, 6.7 Hz), 3.38-3.23 (m, 5H), 2.65 (s, 1H), 2.05 (d, 1H, J=13.4 Hz), 1.97 (d, 1H, J=13.6 Hz). ¹³C NMR (MeOD, 100 MHz): δ 165.8, 157.2, 147.6, 114.0, 107.5, 62.9, 50.8, 49.5, 33.7, 26.7, 24.4. MS (ESI) 221.2 [MH⁺]; HRMS (ESI) calculated for C₁₂H₁₆N₂O₂Na⁺[MNa⁺] 243.1110; found, 243.1109.

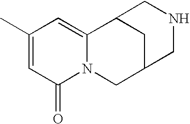

10-Methyl-1,2,3,4,5,6-hexahydro-1,5-methano-pyrido[1,2-a][1,5]diazocin-8-one (15): The above N-Boc protected 10-methyl cytisine derivative 17 (25 mg, 0.09 mmol) in a mixture of TFA/DCM (0.08/0.8) was stirred at room temperature for 2 h. Removed the excess TFA under vacuo, and basified the crude reaction mixture with aqueous ammonia, diluted with ethyl acetate and extracted. The organic layer was dried and concentrated. The crude mass was purified using preparative HPLC (CH₃CN/H₂O mixture in 0.05% TFA). Concentrated to remove the solvent and dried under vacuo to afford 15 mg (87%) of the pure 10-methyl cytisine 15.

Alternative procedure: To a degassed solution of 14, (14 mg, 0.045 mmol) in methanol was added 14 mg of 10% Pd—C and stirred the reaction mixture under 1 atm H₂ pressure overnight (15 h). Filtered the catalyst through a short celite pad, washed with methanol and evaporated. TLC noted. The single product (UV active) was rinsed with hexane, dried and NMR of the crude taken. The product was further purified by HPLC. ¹H NMR (MeOD, 400 MHz): δ 6.35 (s, 1H), 6.31 (s, 1H), 4.15 (d, 1H, J=15.8 Hz), 3.96 (dd, 1H, J=6.7, 9.0 Hz), 3.48-3.37 (m, 5H), 2.76 (s, 1H), 2.23 (s, 3H), 2.17-2.06 (m, 2H). ¹³C NMR (MeOD, 100 MHz): δ 165.6, 153.7, 147.1, 117.4, 111.5, 54.9, 33.2, 26.9, 24.5, 21.2. MS (ESI) 205.1 [MH⁺]; HRMS (ESI) calculated for C₁₂H₁₇N₂O⁺[MH⁺] 205.1341; found, 205.1347.

### Example 3

### Representative Synthetic Scheme for Compounds 19 and 21

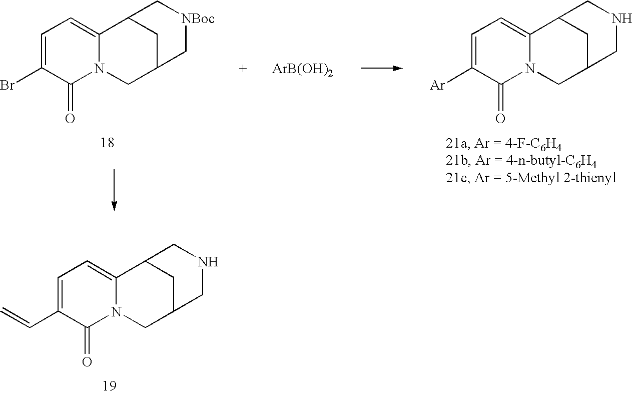

### Synthetic Procedures and Experimental Details for Compounds 19 and 21.

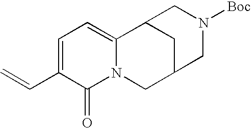

8-Oxo-9-vinyl-1,5,6,8-tetrahydro-2H,4H-1,5-methano-pyrido[1,2-a][1,5]diazocine-3-carboxylic acid tert-butyl ester (19a): Following the procedure reported by Lasne and coworkers (Org. Lett. 2000, 2, 1121-1124), the cross coupling reaction between vinyl tributyl tin (0.105 mL, 0.36 mmol) and N-Boc protected 9-bromo cytisine 18 (89 mg, 0.24 mmol) was carried out in dioxane at 120° C. for 1 h under reflux in presence of catalytic amount of Pd(PPh₃)₂Cl₂. After cooling and removing the solvent under vacuo, saturated aqueous solution of KF (10 mL) was added and stirred the reaction mixture at room temperature for 5 h. After standard work up, the crude product was purified by silica gel column chromatography (MeOH/DCM, 5:95) to get the N-Boc protected 9-vinyl cytisine derivative 19a (56 mg, 73%).

¹H NMR (CDCl₃, 400 MHz): δ 7.41 (d, 1H, J=7.1 Hz), 6.87-6.80 (m, 1H), 6.09 (br s, 1H), 5.96 (d, 1H, J=17.7 Hz), 5.25 (d, 1H, J=11.1 Hz), 4.26-4.22 (m, 3H), 3.85 (dd, 1H, J=9.1, 6.1 Hz), 3.09-3.02 (m, 3H), 2.43 (br s, 1H), 1.98-1.93 (m, 2H), 1.30-1.24 (m, 9H).

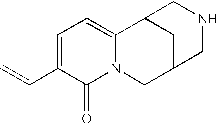

9-Vinyl-1,2,3,4,5,6-hexahydro-1,5-methano-pyrido[1,2-a][1,5]diazocin-8-one (19): 47 mg (0.148 mmol) of the N-Boc protected cytisine 19a in dichloromethane (1 mL) was treated with TFA (0.15 mL) at room temperature for 50 min. Basified with aqueous NH₃ followed by standard aqueous work up gave the crude product which was purified again by HPLC (CH₃CN/water mixture in 0.05% TFA) to get viscous product (19), (26 mg, 81%). [α]²⁵_{D}=−58 (c 0.18, MeOH).

¹H NMR (MeOD, 400 MHz): δ 7.58 (d, 1H, J=7.3 Hz), 6.74 (dd, 1H, J=6.4, 11.3 Hz), 6.32 (d, 1H, J=7.3 Hz), 5.90 (d, 1H, J=17.7 Hz), 5.22 (d, 1H, J=11.3 Hz), 4.14 (d, 1H, J=15.9 Hz), 3.93 (dd, 1H, J=9.2, 6.7 Hz), 3.41-3.26 (m, 5H), 2.69 (br s, 1H), 2.05 (dd, 1H, J=13.3, 17.9 Hz). ¹³C NMR (MeOD, 100 MHz): δ 163.8, 146.9, 136.6, 132.7, 127.4, 116.5, 108.6, 50.9, 49.9, 49.7, 33.3, 26.8, 24.4. MS (ESI) 217.1 [MH⁺]; HRMS (ESI) calculated for C₁₃H₁₇N₂O⁺[MH⁺] 217.1341; found, 217.1342.

### General Procedure for the Suzuki Coupling:

Following the Suzuki coupling procedure by Cosford et al (J. Med. Chem. 2004, 47(19), 4645-4648), the coupling reaction between 9-bromo cytisine 18 and the corresponding boronic acid was carried out. For example, to a mixture of 9-bromo cytisine derivative (18), (17 mg, 0.05 mmol) and 4-n-butyl phenyl boronic acid (11 mg, 0.06 mmol) in DME/H₂O (1:0.2 mL) was added K₂CO₃ (13 mg, 0.09 mmol) and purged of all oxygen by three vacuum/argon cycles. To this mixture under argon was added Pd(PPh₃)₄ followed by one additional vacuum/argon purge cycle. The reaction mixture kept refluxing overnight (15 h). TLC showed complete conversion of the starting cytisine derivative. The reaction mixture was cooled to ambient temperature. After standard work up, the crude product was purified by preparative TLC to get 17 mg (87%) of the coupled product.

Final N-Boc deprotection was carried out using TFA/DCM mixture (30 min, 84%). After usual work up, the product was further purified by HPLC (CH₃CN/water mixture in 0.05% TFA).

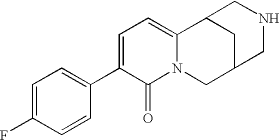

9-(4-Fluorophenyl)-1,2,3,4,5,6-hexahydro-1,5-methano-pyrido[1,2-a][1,5]diazocin-8-one (21a): [α]²⁵_{D}=−43 (c 0.33, MeOH). ¹H NMR (CDCl₃, 400 MHz): δ 7.68 (m, 2H), 7.25-7.48 (m, 1H), 7.08 (t, 2H, J=8.8 Hz), 6.10 (d, 1H, J=7.2 Hz), 4.19 (d, 1H, J=15.7 Hz), 3.97 (dd, 1H, J=6.5, 9.1 Hz), 3.15-2.95 (m, 5H), 2.38 (s, 1H), 1.99 (s, 2H), 1.69 (br s, 1H). ¹³C NMR (CDCl₃, 100 MHz): δ 162.08 (d, J=244.5 Hz), 162.05, 150.4, 136.8, 133.3, (d, J=3.3 Hz), 130.2 (d, J=7.9 Hz), 128.5, 128.4, 126.4, 114.8 (d, J=21.2 Hz), 104.9, 53.9, 53.0, 50.2, 35.7, 27.8, 26.3.

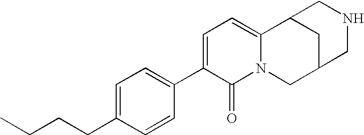

9-(4-Butylphenyl)-1,2,3,4,5,6-hexahydro-1,5-methano-pyrido[1,2-a][1,5]diazocin-8-one (21b). [α]²⁵_{D}=−74 (c 0.14, MeOH). ¹H NMR (CDCl₃, 400 MHz): δ 7.63 (d, 2H, J=7.9 Hz), 7.47 (d, 1H, J=7.2 Hz), 7.22 (d, 2H, J=7.9 Hz), 6.09 (d, 1H, J=7.2 Hz), 4.21 (d, 1H, J=15.7 Hz), 3.970 (dd, 1H, J=6.5, 9.1 Hz), 3.17-3.04 (m, 4H), 2.93 (s, 1H), 2.38 (s, 1H), 1.99 (br s, 2H), 1.42-1.27 (m, 6H), 0.95 (t, 3H, J=7.3 Hz). ¹³C NMR (CDCl₃, 100 MHz): δ 162.2, 141.9, 136.6, 134.6, 128.4, 128.1, 127.5, 104.9, 50.1, 35.4, 33.6, 29.7, 26.3, 22.4, 13.9. MS (ESI) 323.2 [MH⁺]; HRMS (ESI) calculated for C₂₁H₂₇N₂O⁺[MH⁺] 323.2123; found, 323.2128.

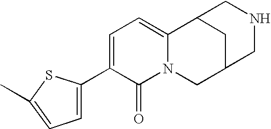

9-(5-Methyl-2-thienyl)-1,2,3,4,5,6-hexahydro-1,5-methano-pyrido[1,2-a][1,5]diazocin-8-one (21c): ¹H NMR (CDCl₃, 400 MHz): δ 7.73 (d, 1H, J=7.4 Hz), 7.42 (d, 1H, J=3.6 Hz), 6.75 (d, 1H, J=2.7 Hz), 6.12 (d, 1H, J=7.4 Hz), 4.28 (d, 1H, J=15.7 Hz), 4.02 (dd, 1H, J=9.5, 6.7 Hz), 3.15-2.95 (m, 5H), 2.53 (s, 3H), 2.37 (s, 1H), 2.00 (s, 2H). ¹³C NMR (CDCl₃, 100 MHz): δ 160.7, 148.6, 140.4, 136.6132.2, 124.7, 123.5, 121.5, 105.1, 52.9, 50.2, 35.7, 29.7, 26.4, 15.2. MS (ESI) 287.1 [MH⁺]; HRMS (ESI) calculated for C₁₆H₁₉N₂OS⁺[MH⁺] 287.1218; found, 287.1217.

### HPLC Purity Analysis Data:

| | Solvent | | t_{R} | | |
|---|---|---|---|---|---|
| Compd No. | system | Wavelength | (min) | Purity | HPLC column |
| rac-15 | A | 280 | 4.0 | 97% | ACE 3 AQ (100 mm × 4.6 mm; |
| | | | | | 3.5 μm) |
| rac-15 | B | 280 | 13.8 | 98% | Supelco LC-18 DB(25 cm × |
| | | | | | 4.6 mm; 5 μm) |
| rac-17a | B | 280 | 11.4 | 99% | Supelco LC-18 DB(25 cm × |
| | | | | | 4.6 mm; 5 μm) |
| rac-17a | A | 280 | 3.1 | 99% | ACE 3 AQ (100 mm × 4.6 mm; |
| | | | | | 3.5 μm) |
| (−)-19 | C | 254 | 7.9 | 98% | ACE 5 AQ (250 mm × 4.6 mm; |
| | | | | | 5 μm) |
| (−)-19 | C | 254 | 11.3 | 99% | Zorbax RX-C18 (150 mm × |
| | | | | | 3 mm, 5 μm) |
| (−)-21a | A | 280 | 7.8 | 98% | Zorbax RX-C18 (150 mm × |
| | | | | | 3 mm, 5 μm) |
| (−)-21a | D | 254 | 6.7 | 97% | ACE 3 AQ (100 mm × 4.6 mm; |
| | | | | | 3.5 μm) |
| (−)-21a | D | 280 | 6.7 | 98% | ACE 3 AQ (100 mm × 4.6 mm; |
| | | | | | 3.5 μm) |
| (−)-21b | A | 254 | 10.9 | 97% | Zorbax RX-C18 (150 mm × |
| | | | | | 3 mm, 5 μm) |
| (−)-21b | C | 254 | 16.5 | 97% | ACE 5 AQ (25 cm × 4.6 mm; |
| | | | | | 5 μm) |
| (−)-21c | A | 360 | 7.4 | 99% | Zorbax RX-C18 (150 mm × |
| | | | | | 3 mm, 5 μm) |
| (−)-21c | E | 360 | 7.4 | 98% | ACE 3 AQ (100 mm × 4.6 mm; |
| | | | | | 3.5 μm) |

Condition A: 2.0 mL/min.; Gradient from 10% acetonitrile in water (0.05% TFA) to 100% acetonitrile (0.05% TFA) in 20 min.
Condition B: 1.3 mL/min.; Gradient from water (0.05% TFA) to 50% acetonitrile (0.05% TFA) in 25 min.
Condition C: 1.3 mL/min.; Gradient from 10% acetonitrile in water (0.05% TFA) to 100% acetonitrile (0.05% TFA) in 20 min.
Condition D: 2.0 mL/min.; Gradient from water (0.05% TFA) to 50% acetonitrile (0.05% TFA) in 15 min and 100% acetonitrile (0.05% TFA) in 22 min.
Condition E: 2.0 mL/min.; Gradient from water (0.05% TFA) to 50% acetonitrile (0.05% TFA) in 11 min and 100% acetonitrile (0.05% TFA) in 18 min.

### Example 4

### Synthetic Procedures and Experimental Details for Compounds 22 and 23

The compounds 22 and 23 were prepared according to the literature procedure (Lasne et al, Tetrahedron: Asymmetry 2002, 13, 1299-1305) and NMR of these matched with the literature.

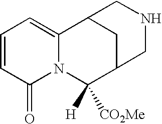

8-Oxo-1,3,4,5,6,8-hexahydro-2H-1,5-methano-pyrido[1,2-a][1,5]diazocine-6-carboxylic acid methyl ester (22): ¹H NMR (CDCl₃, 400 MHz): δ 7.36 (dd, 1H, J=7.0, 1.8 Hz), 6.48 (d, 1H, J=9.0 Hz), 6.07 (d, 1H, J=6.7 Hz), 4.87 (d, 1H, J=5.4 Hz), 3.86-3.72 (m 3H), 3.11 (br s, 2H), 2.90 (s, 3H), 2.49 (br s, 1H), 2.10 (d, 1H, J=12.9 Hz), 2.00-1.92 (m, 2H).

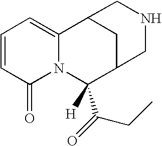

6-Propionyl-1,2,3,4,5,6-hexahydro-1,5-methano-pyrido[1,2-a][1,5]diazocin-8-one (23): ¹H NMR (CDCl₃, 400 MHz): δ 7.34 (dd, 1H, J=6.9, 1.9 Hz), 6.43 (d, 1H, J=8.9 Hz), 6.07 (d, 1H, 6.7 Hz), 5.01 (d, 1H, J=6.6 Hz), 3.09 (br s, 2H), 3.06-2.96 (m, 1H), 2.90 (br s, 1H), 2.80 (br s, 2H), 2.61-2.51 (m, 1H), 2.43 (br s, 1H), 2.11 (d, 1H, J=12.8 Hz), 1.99-1.93 (m, 2H), 1.19 (t, 3H, J=7.1 Hz).

### HPLC Purity Analysis Data:

| Compd | Solvent | | t_{R} | | |
|---|---|---|---|---|---|
| No. | system | Wavelength | (min) | Purity | HPLC column |
| (−)-22* | D | 310 | 5.0 | 96% | ACE 3 AQ |
| | | | | | (100 mm × |
| | | | | | 4.6 mm; 3.5 μm) |
| (−)-23* | D | 310 | 5.6 | 96% | ACE 3 AQ |
| | | | | | (100 mm × |
| | | | | | 4.6 mm; 3.5 μm) |

Condition D: 2.0 mL/min.; Gradient from water (0.05% TFA) to 50% acetonitrile (0.05% TFA) in 15 min and 100% acetonitrile (0.05% TFA) in 22 min.
*Previously reported by Lasne et al, Tetrahedron: Asymmetry 2002, 13, 1299-1305).

### Example 5

### Synthetic Procedures and Experimental Details for Compounds 24-30

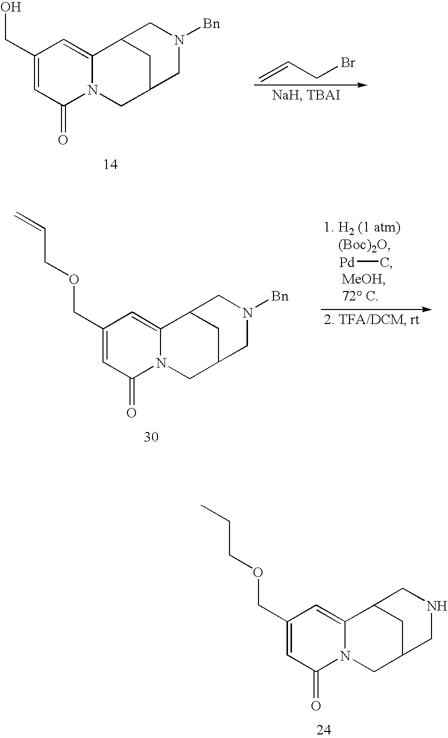

10-(Allyloxymethyl)-3-benzyl-1,2,3,4,5,6-hexahydro-1,5-methano-pyrido[1,2-a][1,5]diazocin-8-one (30): To a stirred solution of the alcohol 14, (20 mg, 0.06 mmol) in dry THF (1 ml) at 0° C. under argon was added NaH (55% by wt., 3 mg, 0.07 mmol). After stirring the mixture for 30 min, a catalytic amount (2.4 mg) of tert-butyl ammonium iodide (TBAI) and allyl bromide (0.01 ml, 0.13 mmol) was added, and the reaction mixture was allowed to warm to room temperature. The reaction was completed in 3.5 h. After cooling, the reaction mixture was quenched with a saturated ammonium chloride solution and the organic layer was extracted with ethyl acetate. The crude product was purified using a semi-preparative HPLC to get 22 mg (97%) of the allyl ether derivative 30. ¹H NMR (CDCl₃, 400 MHz): δ 7.45-7.38 (n 3H), 7.32 (d, 2H, J=6.6 Hz), 6.50 (s, 1H), 6.32 (s, 1H), 5.94-5.86 (m, 1H), 5.32-5.22 (m, 2H0, 4.38-4.15 (m, 5H), 4.03-4.01 (m, 3H), 3.70-3.65 (m, 2H), 3.33 (s, 1H), 3.10 (d, 2H, J=10.6 Hz), 2.83 (s, 1H), 1.99 (br s, 2H). ¹³C NMR (CDCl₃, 100 MHz): δ 152.6, 145.5, 133.5, 131.2, 129.7, 128.8, 127.6, 117.4, 113.4, 107.4, 71.4, 68.9, 60.9, 55.9, 55.5, 48.2, 33.0, 26.1, 23.5

10-(Propoxymethyl)-3-benzyl-1,2,3,4,5,6-hexahydro-1,5-methano-pyrido[1,2-a][1,5]diazocin-8-one (24): To a mixture of the above N-Benzyl allyl ether cytisine derivative 30, (21 mg, 0.06 mmol) and Boc-anhydride (26 mg, 0.12 mmol) in degassed MeOH was added 4.5 mg of 20% Pd(OH)₂—C, degassed three times and refluxed the reaction mixture under 1 atm H₂ pressure for 35 min. Cooled the reaction mixture and the catalyst was removed by filtration, washed with MeOH and concentrated. After the usual aqueous work up with EtOAc and evaporation of the solvent, the crude product was purified using semi-preparative HPLC (CH₃CN/H₂O mixture in 0.05% TFA) to get 8 mg (37%) of N-Boc protected 10-propoxy methyl derivative and 7 mg of the 10-methyl cytisine derivative.

The above N-Boc protected 10-propoxy methyl derivative (6 mg, 0.02 mmol) in a mixture of TFA/DCM (0.02/0.2) was stirred at room temperature for 45 min. Basified the reaction mixture with aqueous ammonia, diluted with ethyl acetate and extracted. The organic layer was dried and concentrated. The crude mass was purified using preparative HPLC (CH₃CN/H₂O mixture in 0.05% TFA). Concentrated and dried under vacuo to afford 3 mg (69%) of the pure 10-propoxymethyl cytisine 24. ¹H NMR (MeOD, 400 MHz): δ 6.44 (s, 1H), 6.31 (s, 1H), 4.36 (s, 2H), 4.09 (d, 1H, J=15.9 Hz), 3.91 (dd, J=9.2, 6.6 Hz), 3.43-3.27 (m, 8H), 2.69 (br s, 1H), 2.05 (dd, 2H, J=14.5, 13.5 Hz), 1.58 (q, 2H, J=7.0 Hz), 0.90 (t, 3H, J=7.4 Hz). ¹³C NMR (CDCl₃, 100 MHz): δ 163.7, 150.7, 150.6, 113.5, 103.8, 72.6, 70.9, 53.9, 52.9, 49.6, 35.7, 27.7, 26.3, 22.8, 10.6.

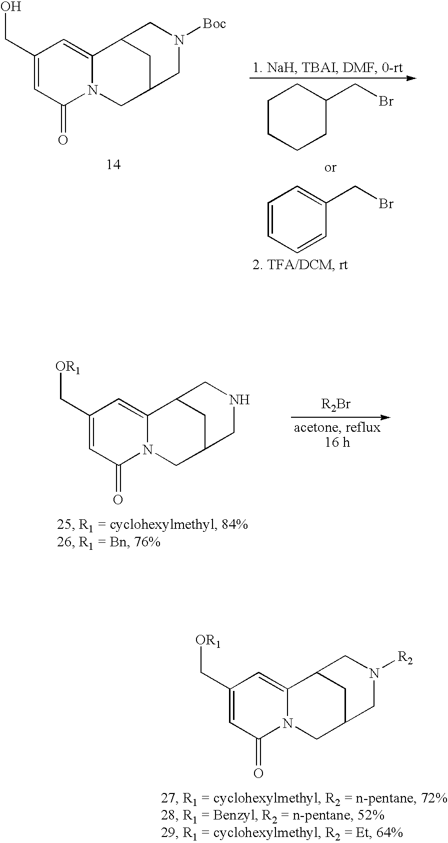

### Representative Experimental Procedure for O-alkylation, N-Boc Deprotection and N-alkylation of Cytisine (25 and 27):

10-Cyclohexyl(methoxymethyl)-1,2,3,4,5,6-hexahydro-1,5-methano-pyrido[1,2-a][1,5]diazocin-8-one (25): To a stirred solution of alcohol 14, (20 mg, 0.06 mmol) in dry THF (1 ml) at 0° C. under argon was added NaH (60% by wt., 7.5 mg, 0.19 mmol). After stirring the mixture for 30 min, a catalytic amount (1.2 mg, 0.003 mmol) of tert-butyl ammonium iodide (TBAI) and bromomethyl-cyclohexane (0.02 ml, 0.14 mmol) was added and the reaction mixture was allowed to warm to room temperature and stir overnight. After cooling, the reaction mixture was quenched with a saturated ammonium chloride solution. The organic layer was extracted with ethyl acetate, dried, and concentrated. The crude product was purified using a semi-preparative HPLC to get 12 mg (46%) of the boc-protected derivative which was further treated with a mixture of TFA/DCM (0.07/1) at ice temperature and slowly warmed to room temperature during 3 h. The crude reaction mixture was basicified with aqueous ammonia and then diluted with ethyl acetate. After aqueous work up, the crude mass was purified using semi-preparative HPLC (CH₃CN/H₂O mixture in 0.05% TFA), to afford 7 mg (84%) of the pure 10-cyclohexyl(methoxymethyl) cytisine 25. ¹H NMR (MeOD, 400 MHz): δ 6.51 (s, 1H), 6.28 (s, 1H), 4.29 (s, 2H), 4.08 (d, 1H, J=5.8 Hz), 3.90 (dd, 1H, J=9.2, 6.7 Hz), 3.40-3.24 (n 6H), 2.68 (br s, 1H), 2.04 (dd, 2H, J=14.8, 13.4 Hz), 1.72-1.52 (m, 6H), 1.22-1.10 (n 5H), 0.96-0.87 (m, 2H). ¹³C NMR (MeOD, 100 MHz): δ 165.9, 154.5, 147.8, 115.1, 107.8, 78.2, 71.7, 51.0, 49.9, 49.7, 39.6, 33.4, 31.3, 27.8, 27.1, 26.8, 24.5.

10-(benzyloxymethyl)-1,2,3,4,5,6-hexahydro-1,5-methano-pyrido[1,2-a][1,5]diazocin-8-one (26): ¹H NMR (CDCl₃, 400 MHz): δ 7.37-7.31 (m, 5H), 6.45 (s, 1H), 6.07 (s, 1H), 4.57 (s, 2H), 4.38 (d, 2H, J=1.9 Hz), 4.13 (d, 1H, J=15.5 Hz), 3.89 (dd, 1H, J=9.0, 6.6 Hz), 3.16-3.02 (m, 4H), 2.93 (s, 1H), 2.36 (br s, 1H), 1.97 (s, 2H). ¹³C NMR (CDCl₃, 100 MHz): δ 163.4, 150.3, 137.7, 128.5, 127.8, 127.7, 113.8, 103.9, 72.6, 70.2, 53.6, 52.7, 49.2, 35.5, 27.6, 26.2.

10-Cyclohexyl(methoxymethyl)-3-pentyl-1,2,3,4,5,6-hexahydro-1,5-methano-pyrido[1,2-a][1,5]diazocin-8-one (27): A mixture of 10-cyclohexyl(methoxymethyl) cytisine 25 (9 mg, 0.03 mmol) and n-pentane bromide (0.002 ml, 0.02 mmol) in dry acetone (1.5 ml) under argon was refluxed overnight. After cooling and removing the solvent under vacuum, the crude mixture was diluted with ethyl acetate. Usual aqueous work up and purification by semi-preparative HPLC afforded 8 mg (72%) of the desired product 27. Some unreacted starting material was also recovered. ¹H NMR (MeOD, 400 MHz): δ 6.51 (s, 1H), 6.37 (d, 1H, J=1.4 Hz), 4.37 (s, 1H), 4.12 (d, 1H, J=15.9 Hz), 3.99 (dd, 1H, J=9.0, 6.8 Hz), 3.67 (d, 1H, J=13.2 Hz), 3.59 (d, 1H, J=12.4 Hz), 3.46 (br s, 1H), 3.37 (br s, 1H), 3.06 (t, 2H, J=8.5 Hz), 2.83 (br s, 1H), 2.09 (d, 2H, J=2.8 Hz), 1.80-1.63 (m, 9H), 1.38-1.24 (n 9H), 1.05-0.9 (m, 2H), 0.92 (t, 3H, J=7.0 Hz)). ¹³C NMR (MeOD, 100 MHz): δ 165.7, 154.4, 147.4, 115.1, 107.6, 78.1, 71.6, 59.8, 59.1, 58.4, 39.5, 34.3, 31.1, 29.7, 27.9, 27.7, 26.9, 24.3, 24.2, 23.1, 14.1.

10-Cyclohexyl(methoxymethyl)-3-ethyl-1,2,3,4,5,6-hexahydro-1,5-methano-pyrido[1,2-a][1,5]diazocin-8-one (29): ¹H NMR (MeOD, 400 MHz): δ 6.53 (s, 1H), 6.38 (d, 1H, J=1.5 Hz), 4.38 (s, 2H), 4.13 (d, 1H, J=15.9 Hz), 4.01 (dd, 1H, J=9.2, 6.6 Hz), 3.69 (d, 1H, J=13.1 Hz), 3.60 (d, 1H, J=12.4 Hz), 3.48-3.47 (m, 1H), 3.35-3.31 (m, 2H), 3.18 (q, 2H, J=7.0 Hz), 2.85 (br s, 1H, 2.11-2.10 (m, 2H), 1.82-1.76 (m, 5H), 1.29-1.22 (m, 9H), 1.06-0.99 (m, 2H). ¹³C NMR (MeOD, 100 MHz): δ 163.9, 152.6, 145.6, 113.3, 105.8, 76.3, 69.8, 56.8, 56.0, 53.3, 37.6, 32.4, 29.3, 26.1, 25.9, 25.2, 22.4, 7.4.

10-(Benzyloxymethyl)-3-pentyl-1,2,3,4,5,6-hexahydro-1,5-methano-pyrido[1,2-a][1,5]diazocin-8-one (28): ¹H NMR (MeOD, 400 MHz): δ 7.39-7.29 (m, 5H), 6.56 (s, 1H), 6.41 (d, 1H, J=1.3 Hz), 4.62 (s, 2H), 4.46 (s, 2H), 4.00 (dd, 1H, J=8.8, 6.6 Hz), 3.67 (d, 1H, J=12.1 Hz), 3.59 (d, 1H, J=12.6), 3.47 (s, 1H), 3.07 (t, 2H, J=8.5 Hz), 2.84 (br s, 1H), 1.66-1.63 (n 2H), 1.38-1.29 (n 6H), 0.92 (t, 3H, J=7.0 Hz). ¹³C NMR (MeOD, 100 MHz): δ 165.7, 154.0, 148.9, 147.6, 129.7, 129.2, 115.5, 107.9, 74.2, 70.9, 59.9, 59.2, 58.5, 34.4, 29.8, 28.1, 24.5, 24.4, 23.3, 14.2.

### INCORPORATION BY REFERENCE

All of the patents and publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A compound represented by formula I: 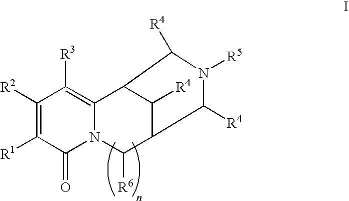
or a pharmaceutically acceptable salt thereof;
wherein
the stereochemical configuration at any stereocenter of said compound is R, S, or a mixture thereof;
R¹, R³, and R⁶ represent independently for each occurrence H, alkyl, cycloalkyl, alkenyl, cycloalkenyl, alkynyl, aryl, aralkyl, halogen, cyano, nitro, —OR⁷, —N(R⁷)₂, —SR⁷, —C(O)R⁷, —CO₂R⁷, —C(O)N(R⁷)₂, —OC(O)R⁷, —N(R⁷)C(O)R⁷, or —(C(R⁸)₂)ₚCR⁸═C(R⁸)₂; wherein said alkyl, cycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷;
R² is alkyl, cycloalkyl, heterocycloalkyl, alkenyl, cycloalkenyl, alkynyl, aryl, aralkyl, halogen, cyano, nitro, —OR⁷, —N(R⁷)₂, —S, —C(O)R⁷, —CO₂R⁷, —C(O)N(R⁷)₂, —OC(O)R⁷, —N(R⁷)C(O)R⁷, —SC(O)R⁷, or —(C(R⁸)₂)ₚCR⁸═C(R⁸)₂; wherein said alkyl, cycloalkyl, heterocycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷; or R² and R³ taken together form a 5-7 member ring containing 0, 1, or 2 heteroatoms selected from the group consisting of O and N; or R¹ and R² taken together form a 5-7 member ring containing 0, 1, or 2 heteroatoms selected from the group consisting of O and N; or R² is 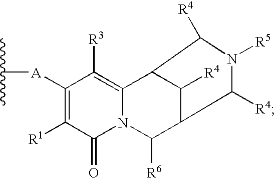
R⁴ represents independently for each occurrence H, alkyl, alkenyl, halogen, —OR⁷, —N(R⁷)₂, or —(C(R⁸)₂)ₚCR⁸═C(R⁸)₂;
R⁵ is H, alkyl, cycloalkyl, alkenyl, cycloalkenyl, alkynyl, aryl, aralkyl, —C(O)R⁷, —CO₂R⁷, —C(O)N(R⁷)₂, or —(C(R⁸)₂)ₚCR⁸═C(R⁸)₂; wherein said alkyl, cycloalkyl, alkenyl, cycloalkenyl, alkynyl, aryl, and aralkyl are optionally substituted with one or more of halogen, —OR⁷, —N(R⁷)₂, —SR⁷, —C(O)R⁷, —CO₂R⁷, —C(O)N(R⁷)₂, —OC(O)R⁷, or —N(R⁷)C(O)R⁷;
R⁷ represents independently for each occurrence H, alkyl, cycloalkyl, heterocycloalkyl, alkenyl, cycloalkenyl, allyl, alkynyl, aryl, aralkyl, alkoxyalkyl, aryloxyalkyl, or cycloalkyloxyalkyl;
R⁸ represents independently for each occurrence H or (C₁-C₆)alkyl;
A is an alkyl diradical, alkenyl diradical, aryl diradical, aralkyl diradical, or —(C(R⁸)₂)ₘ—X—(C(R⁸)₂)ₘ—;
X is O, —N(R⁷)—, or S;
m and p represent independently for each occurrence 1, 2, 3, 4, 5, or 6; and
n is 1 or 2.

2. The compound of claim 1, wherein R¹ represents independently for each occurrence H, alkyl, alkenyl, aryl, aralkyl, halogen, —C(O)R⁷, —CO₂R⁷, —C(O)N(R⁷)₂, or —(C(R⁸)₂)CR⁸═C(R⁸)₂.

3. The compound of claim 1, wherein R¹ represents independently for each occurrence H or (C₁-C₆)alkyl.

4. The compound of claim 1, wherein R¹ represents independently for each occurrence H.

5. The compound of claim 1, wherein R² is alkyl, cycloalkyl, heterocycloalkyl, alkenyl, cycloalkenyl, alkynyl, aryl, aralkyl, halogen, cyano, nitro, —OR⁷, —N(R⁷)₂, —SR⁷, —C(O)R⁷, —CO₂R⁷, —C(O)N(R⁷)₂, —OC(O)R⁷, —N(R⁷)C(O)R⁷, or —(C(R⁸)₂)ₚCR⁸═C(R⁸)₂; wherein said alkyl, cycloalkyl, heterocycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷.

6. The compound of claim 1, wherein R² is alkyl, cycloalkyl, heterocycloalkyl, alkenyl, aryl, aralkyl, halogen, cyano, —C(O)R⁷, —CO₂R⁷, —C(O)N(R⁷)₂, or —(C(R⁸)₂)ₚCR⁸═C(R⁸)₂; wherein said alkyl, cycloalkyl, heterocycloalkyl, alkenyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷.

7. The compound of claim 1, wherein R² is alkyl, cycloalkyl, alkenyl, aryl, or —(C(R⁸)₂)ₚCR⁸═C(R⁸)₂; wherein said alkyl, cycloalkyl, alkenyl, and aryl are optionally substituted with one or more of halogen, —OR⁷, —N(R⁷)₂, or —SR⁷; wherein R⁷ is H or (C₁-C₆)alkyl.

8. The compound of claim 1, wherein R² is alkyl or cycloalkyl; wherein said alkyl and cycloalkyl are optionally substituted with one or more of halogen, —OR⁷, —N(R⁷)₂, or —SR⁷; wherein R⁷ is H or (C₁-C₆)alkyl.

9. The compound of claim 1, wherein R² is (C₁-C₆)alkyl optionally substituted with —OR⁷, —N(R⁷)₂, or —SR⁷; wherein R⁷ is H or (C₁-C₆)alkyl.

10. The compound of claim 1, wherein R² is (C₁-C₆)alkyl optionally substituted with —OR⁷; wherein R⁷ is H or (C₁-C₆)alkyl.

11. The compound of claim 1, wherein R² is (C₁-C₆)alkyl.

12. The compound of claim 1, wherein R² is methyl, ethyl, propyl, isopropyl, butyl, isobutyl, sec-butyl, or pentyl optionally substituted with —OR⁷; wherein R⁷ is H or (C₁-C₆)alkyl.

13. The compound of claim 1, wherein R² is methyl, ethyl, propyl, isopropyl, butyl, isobutyl, sec-butyl, or pentyl.

14. The compound of claim 1, wherein R² is methyl.

15. The compound of claim 1, wherein R² is —CH₂OH.

16. The compound of claim 1, wherein R³ represents independently for each occurrence H, alkyl, alkenyl, aryl, aralkyl, halogen, —C(O)R⁷, —CO₂R⁷, —C(O)N(R⁷)₂, or —(C(R⁸)₂)ₚCR⁸═C(R⁸)₂.

17. The compound of claim 1, wherein R³ represents independently for each occurrence H or (C₁-C₆)alkyl.

18. The compound of claim 1, wherein R³ represents independently for each occurrence H.

19. The compound of claim 1, wherein R⁴ represents independently for each occurrence H or alkyl.

20. The compound of claim 1, wherein R⁴ is H.

21. The compound of claim 1, wherein R⁵ is H, alkyl, cycloalkyl, aryl, aralkyl, —C(O)R⁷, —CO₂R⁷, —C(O)N(R⁷)₂, or —(C(R⁸)₂)ₚCR⁸═C(R⁸)₂.

22. The compound of claim 1, wherein R⁵ is H, alkyl, or benzyl.

23. The compound of claim 1, wherein R⁵ is H.

24. The compound of claim 1, wherein R⁶ represents independently for each occurrence H, alkyl, alkenyl, aryl, aralkyl, halogen, —C(O)R⁷, —CO₂R⁷, —C(O)N(R⁷)₂, or —(C(R⁸)₂)ₚCR⁸═C(R⁸)₂.

25. The compound of claim 1, wherein R⁶ represents independently for each occurrence H or (C₁-C₆)alkyl.

26. The compound of claim 1, wherein R⁶ is H.

27. The compound of claim 1, wherein R⁷ represents independently for each occurrence H, alkyl, cycloalkyl, heterocycloalkyl, alkenyl, cycloalkenyl, allyl, alkynyl, aryl, or aralkyl.

28. The compound of claim 1, wherein R⁷ represents independently for each occurrence H, alkyl, cycloalkyl, aryl, or aralkyl.

29. The compound of claim 1, wherein R⁷ represents independently for each occurrence H, alkoxymethyl, aryloxymethyl, or cycloalkyloxymethyl.

30. The compound of claim 1, wherein R⁷ represents independently for each occurrence H or alkyl.

31. The compound of claim 1, wherein R⁷ is H.

32. The compound of claim 1, wherein n is 1.

33. The compound of claim 1, wherein R¹, R³, R⁴, R⁵, and R⁶ represent independently for each occurrence H or alkyl.

34. The compound of claim 1, wherein R¹, R³, R⁴, R⁵, and R⁶ represent independently for each occurrence H or alkyl; and n is 1.

35-38. (canceled)

39. The compound of claim 1, wherein R¹, R³, R⁴, R⁵, and R⁶ are H; n is 1; R² represents independently for each occurrence methyl, ethyl, propyl, isopropyl, butyl, isobutyl, sec-butyl, or pentyl optionally substituted with —OR⁷; and R⁷ is H or (C₁-C₆)alkyl.

40. The compound of claim 1, wherein R¹, R³, R⁴, R⁵, and R⁶ are H; n is 1; and R² is methyl.

41. The compound of claim 1, wherein R¹, R³, R⁴, R⁵, and R⁶ are H; n is 1; and R² is —CH₂OH.

42-50. (canceled)

51. A compound represented by formula II: 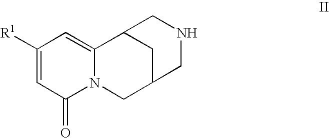
or a pharmaceutically acceptable salt thereof;
wherein
the stereochemical configuration at any stereocenter of said compound is R, S, or a mixture thereof;
R₁ is —OH, —SH, halogen, —CF₃, —CN, —NO₂, optionally substituted C₁-C₆ alkyl chain, optionally substituted benzyl, optionally substituted heteroaryl, optionally substituted cycloalkyl, —NH₂, di-[(C₁-C₆)alkylamino, (C₁-C₆) monoalkylamino, (C₆-C₁₀) arylamino, (C₃-C₈)cycloalkylamino, heteroarylamino, cycloheteroalkylamino; —C(O)R wherein R is H, optionally substituted (C₁-C₆)alkyl, optionally substituted aryl, or optionally substituted benzyl; —CO₂R wherein R is H, (C₁-C₆) alkyl, phenyl, or benzyl; —CON(R)₂ wherein each R is hydrogen, (C₁-C₆)alkyl or (C₆-C₁₀)aryl; —NHC(O)R, wherein R is optionally substituted alkyl (C₁-C₆ chain), optionally substituted aryl, or optionally substituted benzyl; —XR wherein X is O, S or N, and R is hydrogen, alkyl, or aryl bearing 0, 1 or 2 substituents; optionally benzene-fused (C₆-C₁₀) aryl; optionally benzene-fused (C₃-C₈)cycloalkyl; optionally benzene-fused heteroaryl wherein said heteroaryl group contains 5 to 10 atoms comprising one to four heteroatoms; optionally benzene-fused cycloheteroalkyl wherein said cycloheteroalkyl contains 4 to 8 atoms comprising one or two heteroatoms selected from group consisting of N, S and O; —CH₂XR, wherein X is O, S or N, and when X=O, R is selected from the group consisting of hydrogen, allyl, optionally substituted alkenyl, alkoxy methyl, cycloalkyloxy methyl, —C(O)R″ and aryl bearing 0, 1 or 2 substituents, wherein R″ is optionally substituted alkyl, optionally substituted aryl, or optionally substituted benzyl, when X=S or N, R is hydrogen, optionally substituted alkyl, optionally substituted aryl, —NH₂, di-[(C₁-C₆)alkylamino, (C₁-C₆)monoalkylamino, (C₆-C₁₀) arylamino, (C₃-C₈)cycloalkylamino, heteroarylamino, cycloheteroalkylamino or —NHC(O)R′″, wherein R′″ is optionally substituted (C₁-C₆)alkyl chain, optionally substituted aryl, optionally substituted benzyl; —(CH₂)ₙ—OCH₂-(10-Cytisine); —(CH₂)ₙ(10-Cytisine); alkenyl; alkynyl; wherein said alkenyl, alkynyl, and aryl are optionally substituted with halogen, CN, OH, hydroxymethyl, alkoxy, NO₂, amine, alkyl amine, or —NHC(O)R, wherein R is alkyl (C₁-C₆ chain), aryl, or benzyl; and n is 1, 2, 3, 4, 5, or 6.

52. The compound of claim 51, wherein said compound of formula II is a single enantiomer.

53. The compound of claim 51, wherein said compound of formula II is a single diastereomer.

54. A compound represented by formula III: 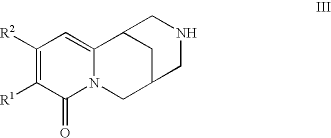
or a pharmaceutically acceptable salt thereof;
wherein
the stereochemical configuration at any stereocenter of said compound is R, S, or a mixture thereof;
R¹ and R² taken together form a 5-8 member ring containing 0, 1, 2, or 3 heteroatoms selected from the group consisting of N, O, and S; and said 5-8 member ring is optionally fused with an aryl or heteroaryl ring.

55. (canceled)

56. A method of modulating a nicotinic ACh receptor in a mammal, comprising the step of administering to a mammal an effective amount of a compound of formula I: 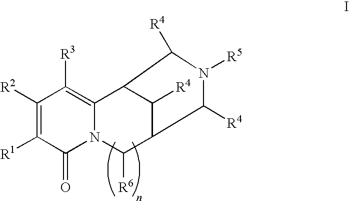
or a pharmaceutically acceptable salt thereof;
wherein
the stereochemical configuration at any stereocenter of said compound is R, S, or a mixture thereof;
R¹, R³, and R⁶ represent independently for each occurrence H, alkyl, cycloalkyl, alkenyl, cycloalkenyl, alkynyl, aryl, aralkyl, halogen, cyano, nitro, —OR⁷, —N(R⁷)₂, —SR⁷, —OC(O)R⁷, —N(R⁷)C(O)R⁷, —SC(O)R⁷, or —(C(R⁸)₂)ₚCR⁸═C(R⁸)₂; wherein said alkyl, cycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷;
R² is alkyl, cycloalkyl, heterocycloalkyl, alkenyl, cycloalkenyl, alkynyl, aryl, aralkyl, halogen, cyano, nitro, —OR⁷, —N(R⁷)₂, —S, —OC(O)R⁷, —N(R⁷)C(O)R⁷, —SC(O)R⁷, —C(O)R⁷, —CO₂R⁷, —C(O)N(R⁷)₂, —OC(O)R⁷, —N(R⁷)C(O)R⁷, or —(C(R⁸)₂)ₚCR⁸═C(R⁸)₂; wherein said alkyl, cycloalkyl, heterocycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷; or R² and R³ taken together form a 5-7 member ring containing 0, 1, or 2 heteroatoms selected from the group consisting of O and N; or R¹ and R² taken together form a 5-7 member ring containing 0, 1, or 2 heteroatoms selected from the group consisting of O and N; or R² is 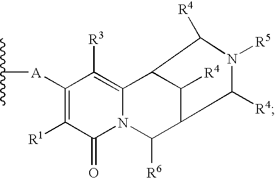
R⁴ represents independently for each occurrence H, alkyl, alkenyl, halogen, —OR⁷, —N(R⁷)₂, or —(C(R⁸)₂)ₚCR⁸═C(R⁸)₂;
R⁵ is H, alkyl, cycloalkyl, alkenyl, cycloalkenyl, alkynyl, aryl, aralkyl, —C(O)R⁷, —CO₂R⁷, —C(O)N(R⁷)₂, or —(C(R⁸)₂)ₚCR⁸═C(R⁸)₂; wherein said alkyl, cycloalkyl, alkenyl, cycloalkenyl, alkynyl, aryl, and aralkyl are optionally substituted with one or more of halogen, —OR⁷, —N(R⁷)₂, —SR⁷, —C(O)R⁷, —CO₂R⁷, —C(O)N(R⁷)₂, —OC(O)R⁷, or —N(R⁷)C(O)R⁷;
R⁷ represents independently for each occurrence H, alkyl, cycloalkyl, heterocycloalkyl, alkenyl, cycloalkenyl, allyl, alkynyl, aryl, aralkyl, alkoxyalkyl, aryloxyalkyl, or cycloalkyloxyalkyl;
R⁸ represents independently for each occurrence H or (C₁-C₆)alkyl;
A is an alkyl diradical, alkenyl diradical, aryl diradical, aralkyl diradical, or —(C(R⁸)₂)ₘ—X—(C(R⁸)₂)ₘ—;
X is O, —N(R⁷)—, or S;
m and p represent independently for each occurrence 1, 2, 3, 4, 5, or 6; and
n is 1 or 2.

57-125. (canceled)

126. A method of modulating a nicotinic ACh receptor in a mammal, comprising the step of administering to a mammal an effective amount of a compound of formula II: 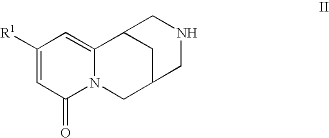
or a pharmaceutically acceptable salt thereof;
wherein
the stereochemical configuration at any stereocenter of said compound is R, S, or a mixture thereof;
R₁ is —OH, —SH, halogen, —CF₃, —CN, —NO₂, optionally substituted C₁-C₆ alkyl chain, optionally substituted benzyl, optionally substituted heteroaryl, optionally substituted cycloalkyl, —NH₂, di-[(C₁-C₆)alkylamino, (C₁-C₆) monoalkylamino, (C₆-C₁₀) arylamino, (C₃-C₈)cycloalkylamino, heteroarylamino, cycloheteroalkylamino; —C(O)R wherein R is H, optionally substituted (C₁-C₆)alkyl, optionally substituted aryl, or optionally substituted benzyl; —CO₂R wherein R is H, (C₁-C₆) alkyl, phenyl, or benzyl; —CON(R)₂ wherein each R is hydrogen, (C₁-C₆)alkyl or (C₆-C₁₀)aryl; —NHC(O)R, wherein R optionally substituted alkyl (C₁-C₆ chain), optionally substituted aryl, or optionally substituted benzyl; —XR wherein X is O, S or N, and R is hydrogen, alkyl, or aryl bearing 0, 1 or 2 substituents; optionally benzene-fused (C₆-C₁₀) aryl; optionally benzene-fused (C₃-C₈)cycloalkyl; optionally benzene-fused heteroaryl wherein said heteroaryl group contains 5 to 10 atoms comprising one to four heteroatoms; optionally benzene-fused cycloheteroalkyl wherein said cycloheteroalkyl contains 4 to 8 atoms comprising one or two heteroatoms selected from group consisting of N, S and O; —CH₂XR, wherein X is O, S or N, and when X=O, R is selected from the group consisting of hydrogen, allyl, optionally substituted alkenyl, alkoxy methyl, cycloalkyloxy methyl, —C(O)R″ and aryl bearing 0, 1 or 2 substituents, wherein R″ optionally substituted alkyl, optionally substituted aryl, or optionally substituted benzyl, when X=S or N, R is hydrogen, optionally substituted alkyl, optionally substituted aryl, —NH₂, di-[(C₁-C₆)alkylamino, (C₁-C₆)monoalkylamino, (C₆-C₁₀) arylamino, (C₃-C₈)cycloalkylamino, heteroarylamino, cycloheteroalkylamino or —NHC(O)R′″, wherein R′″ is optionally substituted (C₁-C₆)alkyl chain, optionally substituted aryl, optionally substituted benzyl; —(CH₂)ₙ—OCH₂-(10-Cytisine); —(CH₂)ₙ(10-Cytisine); alkenyl; alkynyl; wherein said alkenyl, alkynyl, and aryl are optionally substituted with halogen, CN, OH, hydroxymethyl, alkoxy, NO₂, amine, alkyl amine, or —NHC(O)R, wherein R is alkyl (C₁-C₆ chain), aryl, or benzyl; and n is 1, 2, 3, 4, 5, or 6.

127-128. (canceled)

129. A method of modulating a nicotinic ACh receptor in a mammal, comprising the step of administering to a mammal an effective amount of a compound of formula III: 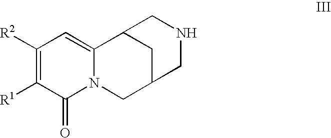
or a pharmaceutically acceptable salt thereof;
wherein
the stereochemical configuration at any stereocenter of said compound is R, S, or a mixture thereof;
R¹ and R² taken together form a 5-8 member ring containing 0, 1, 2, or 3 heteroatoms selected from the group consisting of N, O, and S; and said 5-8 member ring is optionally fused with an aryl or heteroaryl ring.

130. A method of treating a mammal suffering from Alzheimer's disease, Parkinson's disease, dyskinesias, Tourette's syndrome, schizophrenia, attention deficit disorder, anxiety, pain, depression, obsessive compulsive disorder, chemical substance abuse, alcoholism, memory deficit, pseudodementia, Ganser's syndrome, migraine pain, bulimia, obesity, premenstrual syndrome or late luteal phase syndrome, tobacco abuse, post-traumatic syndrome, social phobia, chronic fatigue syndrome, premature ejaculation, erectile difficulty, anorexia nervosa, disorders of sleep, autism, mutism, avoidance learning, or trichotillomania, comprising the step of administering to a mammal in need thereof a therapeutically effective amount of a compound of Formula I: 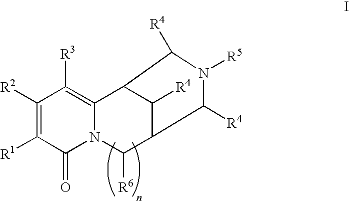
or a pharmaceutically acceptable salt thereof;
wherein
the stereochemical configuration at any stereocenter of said compound is R, S, or a mixture thereof;
R¹, R³, and R⁶ represent independently for each occurrence H, alkyl, cycloalkyl, alkenyl, cycloalkenyl, alkynyl, aryl, aralkyl, halogen, cyano, nitro, —OR⁷, —N(R⁷)₂, —SR⁷, —C(O)R⁷, —CO₂R⁷, —C(O)N(R⁷)₂, —OC(O)R⁷, —N(R⁷)C(O)R⁷, or —(C(R⁸)₂)ₚCR⁸═C(R⁸)₂; wherein said alkyl, cycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷;
R² is alkyl, cycloalkyl, heterocycloalkyl, alkenyl, cycloalkenyl, alkynyl, aryl, aralkyl, halogen, cyano, nitro, —OR⁷, —N(R⁷)₂, —S, —C(O)R⁷, —CO₂R⁷, —C(O)N(R⁷)₂, —OC(O)R⁷, —N(R⁷)C(O)R⁷, —SC(O)R⁷, or —(C(R⁸)₂)ₚCR⁸═C(R⁸)₂; wherein said alkyl, cycloalkyl, heterocycloalkyl, alkenyl, cycloalkenyl, alkynyl, aryl, and aralkyl are optionally substituted with one or more of halogen, nitro, cyano, —OR⁷, —N(R⁷)₂, —SR⁷, —C(O)R⁷, —CO₂R⁷, —C(O)N(R⁷)₂, —OC(O)R⁷, or —N(R⁷)C(O)R⁷; or R² and R³ taken together form a 5-7 member ring containing 0, 1, or 2 heteroatoms selected from the group consisting of O and N; or R¹ and R² taken together form a 5-7 member ring containing 0, 1, or 2 heteroatoms selected from the group consisting of O and N; or R² is 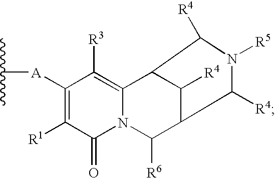
R⁴ represents independently for each occurrence H, alkyl, alkenyl, halogen, —OR⁷, —N(R⁷)₂, or —(C(R⁸)₂)ₚCR⁸═C(R⁸)₂;
R⁵ is H, alkyl, cycloalkyl, alkenyl, cycloalkenyl, alkynyl, aryl, aralkyl, —C(O)R⁷, —CO₂R⁷, —C(O)N(R⁷)₂, or —(C(R⁸)₂)ₚCR⁸═C(R⁸)₂; wherein said alkyl, cycloalkyl, alkenyl, cycloalkenyl, alkynyl, aryl, and aralkyl are optionally substituted with one or more of halogen, —OR⁷, —N(R⁷)₂, —SR⁷, —C(O)R⁷, —CO₂R⁷, —C(O)N(R⁷)₂, —OC(O)R⁷, or —N(R⁷)C(O)R⁷;
R⁷ represents independently for each occurrence H, alkyl, cycloalkyl, heterocycloalkyl, alkenyl, cycloalkenyl, allyl, alkynyl, aryl, aralkyl, alkoxyalkyl, aryloxyalkyl, or cycloalkyloxyalkyl;
R⁸ represents independently for each occurrence H or (C₁-C₆)alkyl;
A is an alkyl diradical, alkenyl diradical, aryl diradical, aralkyl diradical, or —(C(R⁸)₂)ₘ—X—(C(R⁸)₂)ₘ—;
X is O, —N(R⁷)—, or S;
m and p represent independently for each occurrence 1, 2, 3, 4, 5, or 6; and
n is 1 or 2.

131-193. (canceled)

194. A method of treating a mammal suffering from Alzheimer's disease, Parkinson's disease, dyskinesias, Tourette's syndrome, schizophrenia, attention deficit disorder, anxiety, pain, depression, obsessive compulsive disorder, chemical substance abuse, alcoholism, memory deficit, pseudodementia, Ganser's syndrome, migraine pain, bulimia, obesity, premenstrual syndrome or late luteal phase syndrome, tobacco abuse, post-traumatic syndrome, social phobia, chronic fatigue syndrome, premature ejaculation, erectile difficulty, anorexia nervosa, disorders of sleep, autism, mutism, avoidance learning, or trichotillomania, comprising the step of administering to a mammal in need thereof a therapeutically effective amount of a compound of Formula II: 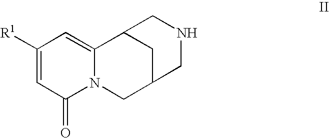
or a pharmaceutically acceptable salt thereof;
wherein
the stereochemical configuration at any stereocenter of said compound is R, S, or a mixture thereof;
R¹ is —OH, —SH, halogen, —CF₃, —CN, —NO₂, optionally substituted C₁-C₆ alkyl chain, optionally substituted benzyl, optionally substituted heteroaryl, optionally substituted cycloalkyl, —NH₂, di-[(C₁-C₆)alkylamino, (C₁-C₆) monoalkylamino, (C₆-C₁₀) arylamino, (C₃-C₈)cycloalkylamino, heteroarylamino, cycloheteroalkylamino; —C(O)R wherein R is H, optionally substituted (C₁-C₆)alkyl, optionally substituted aryl, or optionally substituted benzyl; —CO₂R wherein R is H, (C₁-C₆) alkyl, phenyl, or benzyl; —CON(R)₂ wherein each R is hydrogen, (C₁-C₆)alkyl or (C₆-C₁₀)aryl; —NHC(O)R, wherein R optionally substituted alkyl (C₁-C₆ chain), optionally substituted aryl, or optionally substituted benzyl; —XR wherein X is O, S or N, and R is hydrogen, alkyl, or aryl bearing 0, 1 or 2 substituents; optionally benzene-fused (C₆-C₁₀) aryl; optionally benzene-fused (C₃-C₈)cycloalkyl; optionally benzene-fused heteroaryl wherein said heteroaryl group contains 5 to 10 atoms comprising one to four heteroatoms; optionally benzene-fused cycloheteroalkyl wherein said cycloheteroalkyl contains 4 to 8 atoms comprising one or two heteroatoms selected from group consisting of N, S and O; —CH₂XR, wherein X is O, S or N, and when X=O, R is selected from the group consisting of hydrogen, allyl, optionally substituted alkenyl, alkoxy methyl, cycloalkyloxy methyl, —C(O)R″ and aryl bearing 0, 1 or 2 substituents, wherein R″ optionally substituted alkyl, optionally substituted aryl, or optionally substituted benzyl, when X=S or N, R is hydrogen, optionally substituted alkyl, optionally substituted aryl, —NH₂, di-[(C₁-C₆)alkylamino, (C₁-C₆)monoalkylamino, (C₆-C₁₀) arylamino, (C₃-C₈)cycloalkylamino, heteroarylamino, cycloheteroalkylamino or —NHC(O)R′″, wherein R′″ is optionally substituted (C₁-C₆)alkyl chain, optionally substituted aryl, optionally substituted benzyl; —(CH₂)ₙ—OCH₂-(10-Cytisine); —(CH₂)ₙ(10-Cytisine); alkenyl; alkynyl; wherein said alkenyl, alkynyl, and aryl are optionally substituted with halogen, CN, OH, hydroxymethyl, alkoxy, NO₂, amine, alkyl amine, or —NHC(O)R, wherein R is alkyl (C₁-C₆ chain), aryl, or benzyl; and n is 1, 2, 3, 4, 5, or 6.

195. (canceled)

196. A method of treating a mammal suffering from Alzheimer's disease, Parkinson's disease, dyskinesias, Tourette's syndrome, schizophrenia, attention deficit disorder, anxiety, pain, depression, obsessive compulsive disorder, chemical substance abuse, alcoholism, memory deficit, pseudodementia, Ganser's syndrome, migraine pain, bulimia, obesity, premenstrual syndrome or late luteal phase syndrome, tobacco abuse, post-traumatic syndrome, social phobia, chronic fatigue syndrome, premature ejaculation, erectile difficulty, anorexia nervosa, disorders of sleep, autism, mutism, avoidance learning, or trichotillomania, comprising the step of administering to a mammal in need thereof a therapeutically effective amount of a compound of Formula III: 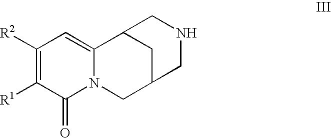
or a pharmaceutically acceptable salt thereof;
wherein
the stereochemical configuration at any stereocenter of said compound is R, S, or a mixture thereof;
R¹ and R² taken together form a 5-8 member ring containing 0, 1, 2, or 3 heteroatoms selected from the group consisting of N, O, and S; and said 5-8 member ring is optionally fused with an aryl or heteroaryl ring.

197-198. (canceled)
